(19) United States
(12) Patent Application Publication
(10) Pub. No.: US 2017/0360570 A1
(43) Pub. Date: Dec. 21, 2017

(54) APPARATUS AND METHODS FOR SPINE AND SACROILIAC JOINT REPAIR

(71) Applicant: CONVENTUS ORTHOPAEDICS, INC., Maple Grove, MN (US)

(72) Inventors: Jonathan Berndt, Crystal, MN (US); Steve D. Kruse, St. Michael, MN (US); Todd A. Krinke, Buffalo, MN (US); Michael P. Brenzel, St. Paul, MN (US); Paul Hindrichs, Plymouth, MN (US)

(21) Appl. No.: 15/439,326

(22) Filed: Feb. 22, 2017

Related U.S. Application Data
(60) Provisional application No. 62/298,421, filed on Feb. 22, 2016.

(51) Int. Cl. A61F 2/44; A61B 17/00; A61B 17/56; A61F 2/46; A61B 17/70

(52) U.S. Cl. CPC A61F 2/442; A61F 2/4455; A61F 2/446; A61F 2/4611; A61B 17/7059; A61B 17/7032; A61B 2017/564; A61B 2017/00526; A61F 2002/448; A61F 2002/4495

(57) ABSTRACT

Apparatus and methods for treating a spinal body having an interior. The method may include augmenting a height of the spinal body by radially expanding a first mesh cage in the interior. The method may include removing the first cage from the interior. The method may include supporting the spinal body in an elevated position by radially expanding a second mesh cage in the interior. The method may also include surgically enclosing the second cage in the interior.

### CROSS REFERENCE TO RELATED APPLICATIONS

This application is a nonprovisional of U.S. Provisional Application No. 62/298,421, filed on Feb. 22, 2016, which is hereby incorporated herein by reference in its entirety.

### BACKGROUND

Orthopedic therapy for the spine may involve creation or restoration of appropriate positioning, and securing, of anatomical members such as vertebrae, intervertebral discs and other spine-related tissue.

The therapy may involve delivering to the tissue structures that counteract or partially counteract forces on the spine-related tissue.

Placement of the structures may be important for proper healing of the bone.

It would be desirable, therefore, to provide apparatus and methods for positioning spine-related tissue.

It would be desirable, therefore, to provide apparatus and methods for securing spine-related tissue.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 shows an illustrative method in accordance with principles of the invention;

FIG. 2 shows a portion of a human skeleton;

FIG. 3 shows an illustrative view of a portion of a spine;

FIG. 4 shows a top plan view of an illustrative vertebra;

FIG. 5 shows an illustrative view of a portion of a spine;

FIG. 6 shows an illustrative view of a portion of a spine;

FIG. 7 shows illustrative apparatus and methods in accordance with the principles of the invention;

FIG. 8 shows illustrative apparatus and methods in accordance with the principles of the invention;

FIGS. 9-17 show illustrative apparatus and steps of a method in accordance with the principles of the invention;

FIGS. 18-29 show illustrative apparatus and steps of a method in accordance with the principles of the invention;

FIGS. 30-41 show illustrative apparatus and steps of a method in accordance with the principles of the invention

FIGS. 42-48 show illustrative apparatus and steps of a method in accordance with the principles of the invention;

FIGS. 49-60 show illustrative apparatus and steps of a method in accordance with the principles of the invention;

FIGS. 61-69 show illustrative apparatus and steps of a method in accordance with the principles of the invention;

FIG. 70 shows illustrative apparatus in accordance with the principles of the invention;

FIG. 71 shows illustrative apparatus in accordance with the principles of the invention;

FIG. 72 shows illustrative apparatus in accordance with the principles of the invention;

FIGS. 73-78 show illustrative apparatus and steps of a method in accordance with the principles of the invention;

FIG. 79 shows illustrative apparatus in accordance with the principles of the invention;

FIG. 80 shows illustrative apparatus in accordance with the principles of the invention;

FIGS. 81-88 show illustrative apparatus and steps of a method in accordance with the principles of the invention;

FIG. 89 shows an illustrative view of a portion of a human skeleton;

FIG. 90 shows illustrative apparatus in accordance with the principles of the invention;

FIG. 91 shows illustrative apparatus in accordance with the principles of the invention;

FIGS. 92-97 show illustrative apparatus and steps of a method in accordance with the principles of the invention;

FIG. 98 shows illustrative method and apparatus in accordance with principles of the invention;

FIGS. 99-108 show illustrative apparatus and steps of a method in accordance with the principles of the invention;

FIG. 109 shows illustrative shapes of a cage in accordance with the principles of the invention;

FIG. 110 shows illustrative methods and apparatus in accordance with principles of the invention;

FIG. 111 shows illustrative methods and apparatus in accordance with principles of the invention;

FIG. 112 shows illustrative apparatus in accordance with the invention; and

FIG. 113 shows illustrative apparatus in accordance with the invention.

### DETAILED DESCRIPTION

Apparatus and methods for treating a spinal body are provided. The spinal body may be a vertebra. The spinal body may be an intervertebral disc.

Apparatus and methods for reducing a gap between a first body tissue and a second body tissue are provided.

Methods described herein may be performed during a minimally-invasive procedure.

The methods, which may involve the apparatus shown and described herein, may include methods for providing orthopedic therapy to a patient. The apparatus may be used to provide orthopedic therapy to the patient.

The apparatus and methods described herein may be used to treat degenerative disc disease, spinal stenosis, spondylosis, spondylolisthesis, pars interarticularis, kyphosis, herniation, a bulging disc, a thinning disc, disc degeneration, sciatica, trauma including a compression fracture, a tumor, scoliosis, or any other condition of a spinal body.

The apparatus and methods described herein may augment a height of a vertebra. The apparatus and methods described herein may support a vertebra without augmenting the height. The apparatus and methods may be used during a kypholasty procedure. The apparatus and methods may be used during a vertebralplasty procedure.

The methods may be used to treat the spinal body in a unilateral procedure. The methods may be used to treat the spinal body in a bilateral procedure.

The methods may be performed using an extrapedicular approach. An extrapedicular approach may access the spinal body without passing through the pedicle. An extrapedicular approach may be performed along an access path that does not transect the pedicle.

The methods may be performed using a transpedicular approach. A transpedicular approach may access the spinal body through the pedicle. A transpedicular approach may be performed along an access path that passes through the pedicle. A transpedicular approach may be performed along an access path that transects the pedicle.

The methods may be performed using both an extrapedicular approach and a transpedicular approach.

The methods may include performing a laminectomy. The methods may include performing a laminotomy.

The methods may be used to treat a single spinal body.

When the spinal body is a vertebra, the methods may be used to treat two vertebrae, three vertebras, four vertebras or five or more vertebras. The vertebras may be adjacent. At least one of the vertebras being treated may not be adjacent other vertebras being treated.

When the spinal body is an intervertebral disc, the methods may be used to treat two intervertebral discs, three intervertebral discs, four intervertebral discs or five or more intervertebral discs. The intervertebral discs may be adjacent. At least one of the intervertebral discs being treated may not be adjacent other intervertebral discs being treated.

The methods may be used to treat both an intervertebral disc and a vertebra during a procedure. The intervertebral disc may be adjacent the vertebra. The intervertebral disc may not be adjacent the vertebra.

The methods, which may involve the apparatus shown and described herein, may include methods for treating a spinal body. The spinal body may have an interior. The spinal body may be a vertebra. The spinal body may be an intervertebral disc. The methods may include one or more steps from any other methods disclosed herein.

The spinal body may be a first spinal body. The first spinal body may be disposed between a second spinal body and a third spinal body. The first spinal body may be disposed adjacent the second spinal body.

When the spinal body is a vertebra, the vertebra may be disposed between a first intervertebral disc and a second intervertebral disc. When the spinal body is an intervertebral disc, the intervertebral disc may be disposed between a first vertebra and a second vertebra.

The methods may include augmenting a height of the spinal body. The augmenting may include expanding a cage. The augmenting may include patient positioning. The augmenting may include using a surgical table. The augmenting may include two or more of the aforementioned methods.

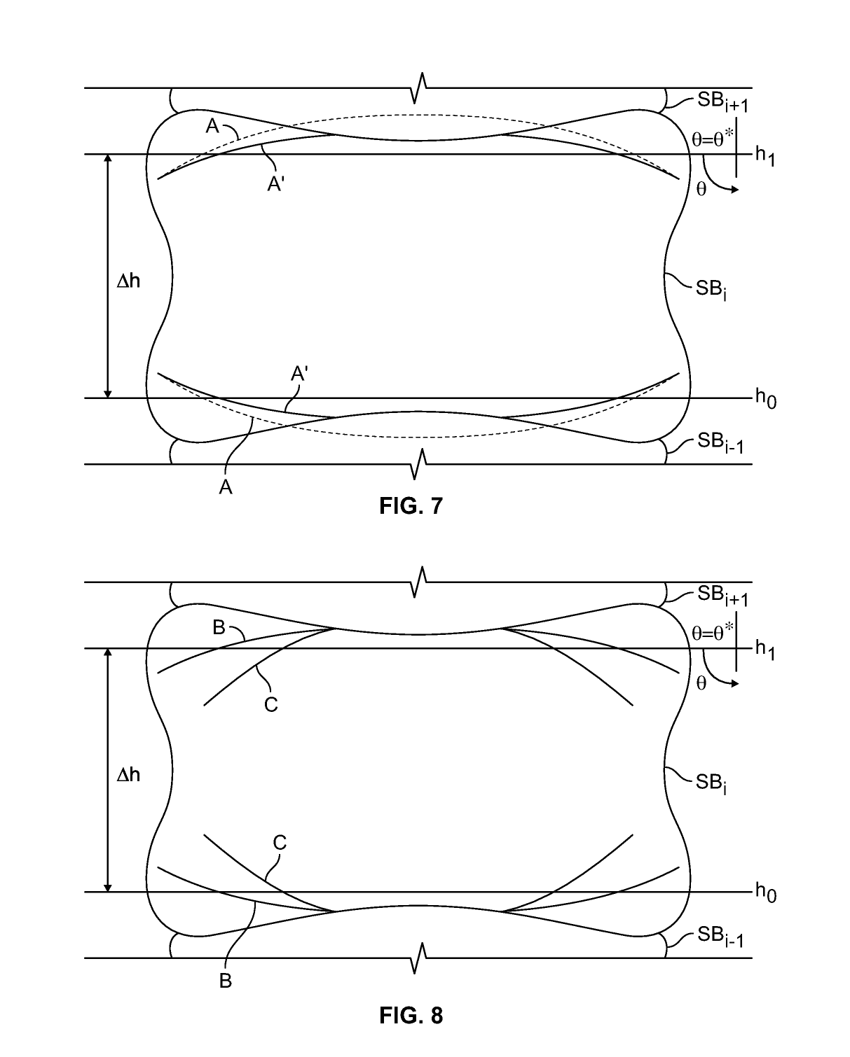

The expansion of the cage may deform the cage under a load applied to the cage during the expansion. Illustrative deformation of the cage, when the cage is a single-layer cage, under a load applied during expansion is illustrated in FIG. 7. Illustrative deformation of the cage, when the cage is a double-layered cage, under a load applied during expansion is illustrated in FIG. 8.

The augmenting may include decompressing impingement of a nerve. The nerve may extend along an outer face of the spinal body. The outer face may be a posterior face of the spinal body.

The cage may be a first cage. The first cage may be referred to alternately herein as a “mesh cage.”

The cage may be radially expanded. The cage may be a mesh cage. The mesh cage may be formed from a laser-cut tube. The mesh cage, when expanded, may form a mesh. The mesh may include a plurality of interconnected cells. The cage may include one or more features of apparatus and methods described in U.S. patent application Ser. No. 12/353,855, filed on Jan. 14, 2009, now U.S. Pat. No. 8,287,538, U.S. patent application Ser. No. 13/043,190, filed on Mar. 8, 2011, now U.S. Pat. No. 8,906,022, U.S. patent application Ser. No. 13/945,137, filed on Jul. 18, 2013, and/or U.S. patent application Ser. No. 14/568,301, filed on Dec. 12, 2014, all of which are hereby incorporated by reference herein in their entireties.

The cage may include a first cell. The cage may include a second cell. The second cell may be positioned apart from the first cell. The cage may include a plurality of cells. Some or all of the plurality of cells may be shaped to receive screws or any other suitable anchors.

The shapes of the configuration of the cage, when expanded, may impact one or both of a radial or hoop strength of the cage. Cell patterns, wall thickness, and strut dimensions of the cage may be varied to enhance the radial or hoop strength of the desired geometry. The cage may include cell patterns. The cell patterns may vary along a length of the cage. The cell patterns may vary about a circumference of the cage to aide in creation of a desired shape and stiffness profile. The cage may be strengthened by adding additional layers. The additional layers may exhibit a different shape than the outer layer. The different shapes may provide enhanced radial or hoop force, thus adding radial or hoop strength to the cage. An outer layer of the cage may have a concave profile to best match an anatomy within which the cage will be expanded. An inner layer of the cage may be mainly round and symmetric to maximize the strength of the first cage.

Expanding the cage may include manually expanding the cage. The cage may be manually expanded using a mechanical actuation mechanism. Expanding the cage may include expanding the cage using an electrical actuation mechanism. Any suitable actuation mechanism known to those skilled in the art may be used to expand the cage.

A neutral state of the cage may be an expanded state. Expanding the cage may include advancing the cage out of a delivery sheath. The cage may be advanced out of the delivery sheath and into the interior. The cage may self-expand to occupy a first volume. The cage may self-expand while being advanced out of the delivery sheath. The cage may self-expand after being advanced out of the delivery sheath. Expanding first cage may also include using an actuation mechanism to expand the cage from the first volume to a second volume. The second volume may be greater than the first volume.

The cage may self-expand using a spring mechanism. Expanding the cage may include advancing the cage out of a delivery sheath. The cage may be advanced out of the delivery sheath and into the interior. The cage may self-expand to occupy a first volume. The cage may self-expand while being advanced out of the delivery sheath. The cage may expand after being advanced out of the delivery sheath. Expanding may also include using an actuation mechanism to expand the cage from the first volume to a second volume. The second volume may be greater than the first volume.

The cage may not be used to prepare a cavity in the interior. The methods may include not rotating the cage. The methods may include not rotating the cage to displace bone material. The methods may include not using the cage as an implant to support the spinal body in an elevated state. The cage may not include a cutting edge.

The cage may be used to prepare a cavity in the interior. The methods may include rotating the cage. The cage may include a cutting edge.

The cage may be formed from any suitable material such as stainless steel, Nitinol, other alloy, titanium alloys, aluminum alloys, composites of carbon fiber, or one or more plastics or epoxy resin. A shape of the cage may be thermally set or set by thermal deformation.

The cage may have a taper as viewed from the side. The broaching member may be shaped to provide a cavity having a taper that accommodates the cage taper. The taper angle τ is defined on an illustrative taper that is bisected by a horizontal plane. τ ranges from τ/2 below the plane to τ/2 above the plan. Table 1 shows illustrative taper angle ranges.

TABLE 1
Illustrative taper angle ranges.
Illustrative taper angle ranges (lower and upper limits, inclusive) (°)

| Lower | Upper | Lower | Upper | Lower | Upper |
|---|---|---|---|---|---|
| <1 | 2 | <1 | 3 | <1 | 5 |
| 2 | 3 | 3 | 5 | 5 | 10 |
| 3 | 4 | 5 | 7 | 10 | 15 |
| 4 | 5 | 7 | 9 | 15 | 20 |
| 5 | 6 | 9 | 11 | 20 | 25 |
| 6 | 7 | 11 | 13 | 25 | 30 |
| 7 | 8 | 13 | 15 | 30 | 35 |
| 8 | 9 | 15 | 17 | 35 | 40 |
| 9 | 10 | 17 | 19 | 40 | >45 |
| 10 | 11 | 19 | 21 | | |
| 11 | 12 | 21 | 23 | | |
| 12 | 13 | 23 | 25 | | |
| 13 | 14 | 25 | 27 | | |
| 14 | 15 | 27 | 29 | | |
| 15 | 16 | 29 | 31 | | |
| 16 | 17 | 31 | 33 | | |
| 17 | 18 | 33 | 35 | | |
| 18 | 19 | 35 | 37 | | |
| 19 | 20 | 37 | 39 | | |
| 20 | 21 | 39 | 41 | | |
| 21 | 22 | 41 | 43 | | |
| 22 | 23 | 43 | >45 | | |
| 23 | 24 | | | | |
| 24 | 25 | | | | |
| 25 | 26 | | | | |
| 26 | 27 | | | | |
| 27 | 28 | | | | |
| 28 | 29 | | | | |
| 29 | 30 | | | | |
| 30 | 31 | | | | |
| 31 | 32 | | | | |
| 32 | 33 | | | | |
| 33 | 34 | | | | |
| 34 | 35 | | | | |
| 35 | 36 | | | | |
| 36 | 37 | | | | |
| 37 | 38 | | | | |
| 38 | 39 | | | | |
| 39 | 40 | | | | |
| 40 | 41 | | | | |
| 41 | 42 | | | | |
| 42 | 43 | | | | |
| 43 | 44 | | | | |
| 44 | >45 | | | | |

The methods may include removing the cage from the interior. The cage may be removed by collapsing the cage and withdrawing cage from the interior. When the methods do not include implanting the cage in the interior, the cage may be formed from non-implantable material. Exemplary non-implantable material may include stainless steel.

The methods may include, after expanding the cage in the interior, removing a handle of the cage. The methods may include, after expanding the cage in the interior, surgically enclosing the cage in the interior. Fixation mechanisms described herein may be used to support anatomy surrounding the implanted cage. An interior of the cage may or may not be filled with bone graft, morcellated bone, or bone cement.

The cage may be a first cage. The methods may include supporting the spinal body. The supporting may be performed after the augmenting. The supporting may be performed after removing the first cage from the interior. The supporting may be performed using a second cage.

The second cage may have one or more features in common with the first cage. The first cage may have one or more features in common with the second cage. The second cage may be referred to alternately herein as a “second mesh cage.”

The spinal body may be supported in an elevated position. The elevated position may be achieved by expanding the second cage. The elevated position may be achieved by positioning the patient. The elevated position may be achieved using a surgical table. The elevated position may be achieved by two or more of the aforementioned methods.

The spinal body may be supported in the elevated position by expanding a second cage in the interior. The second cage may be a second mesh cage. The second mesh cage may be formed from a laser-cut tube. The second mesh cage, when expanded, may form a mesh. The mesh may include a plurality of interconnected cells. The second cage, and any other cage disclosed herein, may include one or more features of apparatus and methods described in U.S. patent application Ser. No. 12/353,855, Ser. No. 13/043,190, Ser. No. 14/568,301 and Ser. No. 13/945,137, which have all been incorporated by reference herein in their entireties.

The second mesh cage may include a first cell. The second mesh cage may include a second cell. The second cell may be positioned apart from the first cell. The second mesh cage may include a plurality of cells. Some or all of the plurality of cells may be shaped to receive screws or any other suitable anchors.

The second cage may be formed of implantable material. The second cage may be used as an implant. The second cage may not include a cutting edge.

Expanding the second cage may include manually expanding the second cage using a mechanical actuation mechanism. Expanding the second mesh cage may include expanding the second cage using an electrical actuation mechanism. Any suitable actuation mechanism known to those skilled in the art may be used to expand the second cage.

The first cage may be expanded in the interior to occupy a first volume. The second cage may be expanded in the interior to occupy the first volume. The second cage may be expanded to the first volume. The second cage may be expanded to a second volume different from the first volume. The second volume may be larger than the first volume. The second volume may be smaller than the first volume.

A neutral state of the second cage may be an expanded state. Expanding the second cage may include advancing the second cage out of a delivery sheath. The second cage may be advanced out of the delivery sheath and into the interior. The second cage may self-expand to occupy a second volume. The second cage may self-expand while being advanced out of the delivery sheath. The second cage may expand after being advanced out of the delivery sheath. Expanding the second cage may also include using an actuation mechanism to expand the second cage from the first volume to a second volume. The second volume may be greater than the first volume.

The second cage may self-expand using a spring mechanism. Expanding the second cage may include advancing the second cage out of a delivery sheath. The second cage may be advanced out of the delivery sheath and into the interior. The second cage may self-expand to occupy a second volume. The second cage may self-expand while being advanced out of the delivery sheath. The second cage may expand after being advanced out of the delivery sheath. Expanding may also include using an actuation mechanism to expand the second cage from the first volume to a second volume. The second volume may be greater than the first volume

The methods may include surgically enclosing the second cage in the interior. The surgically enclosing may include closing an access hole prepared by a practitioner to access the interior. The access hole may be positioned on a surface of the patient's body.

When the spinal body is an intervertebral disc, the augmenting may include positioning the first mesh cage against the first vertebra and the second vertebra. The augmenting may include positioning the first mesh cage against the intervertebral disc. The augmenting may include positioning the first mesh cage against the first vertebra and the intervertebral disc.

When the spinal body is an intervertebral disc, the supporting may include positioning the second mesh cage against the first vertebra and the second vertebra. The supporting may include positioning the second mesh cage against the intervertebral disc. The supporting may include positioning the second mesh cage against the first vertebra and the intervertebral disc.

When spinal body is a vertebra, the augmenting may include positioning the first mesh cage against the vertebra. The supporting may include positioning the second mesh cage against the vertebra.

The first mesh cage may have a maximum radius in the collapsed state. The first mesh cage may have a maximum radius in the expanded state. A ratio between the maximum radius in the collapsed state and the maximum radius in the expanded state may be no less than one to three. The ratio between the maximum radius in the collapsed state and the maximum radius in the expanded state may be no less than one to four. The ratio may have a lower and upper limit. Exemplary ratios having lower and upper limits are included in Table 2, below.

The second mesh cage may have a maximum radius in the collapsed state. The second mesh cage may have a maximum radius in the expanded state. A ratio between the maximum radius in the collapsed state and the maximum radius in the expanded state may be no less than one to three. A ratio between the maximum radius in the collapsed state and the maximum radius in the expanded state may be no less than one to four. The ratio may have a lower and upper limit. Exemplary ratios having lower and upper limits are included in Table 2, below.

TABLE 2
Selected ratios.
Ratio of maximum radius, collapsed
state vs. expanded state, illustrative
ranges (lower and upper limits,
inclusive)

| Lower | Upper |
|---|---|
| 2.0 | 2.2 |
| 2.2 | 2.4 |
| 2.4 | 2.6 |
| 2.6 | 2.8 |
| 2.8 | 3.0 |
| 3.0 | 3.2 |
| 3.2 | 3.4 |
| 3.4 | 3.6 |
| 3.6 | 3.8 |
| 3.8 | 4.0 |
| 4.0 | 4.2 |
| 4.2 | 4.4 |
| 4.4 | 4.6 |
| 4.6 | 4.8 |
| 4.8 | 5.0 |
| 5.0 | 5.2 |
| 5.2 | 5.4 |
| 5.4 | 5.6 |
| 5.6 | 5.8 |
| 5.8 | 6.0 |
| 6.0 | >6.0 |

The methods may include preparing an access hole on a surface of the patient's body. The methods may include preparing an access path between the access hole and the interior. Preparing the access path may include advancing a guide wire through the access hole and into the interior.

The methods may include positioning a tip of the guide wire at a target site in the interior. The access path may extend between the access hole and the target site. The guide wire may be a k-wire. The guide wire may be a probe. The guide wire may detect electronic impulses generated by a spinal cord.

The target site may be a desired location for positioning a distal end of a drill in the interior. The target site may be a desired location for positioning a distal end of the first cage in the interior. The target site may be a desired location for positioning a distal end of a cavity preparation instrument in the interior. The target site may be a desired location for positioning a distal end of the second cage in the interior.

For the purposes of the application, the terms “proximal” and “distal” may be defined relative to a practitioner. A proximal end of an apparatus may be an end of the apparatus closest to the practitioner. A distal end of the apparatus may be an end of the apparatus furthest away from the practitioner.

The methods may include augmenting a diameter of the access path. The augmenting may include drilling along the guide wire to the target site. The access path may be straight. The access path may be curved. The access path may include one or more curved sections and one or more straight sections. The augmenting may include using one or more retractor tubes, hand retractors, and/or tube dilators.

The methods may include advancing the first cage along the access path and into the interior. The methods may include advancing the second cage along the access path and into the interior, after removal of the first cage from the interior. The first cage may be positioned inside a first sheath and advanced along the access path and into the interior. The second cage may be positioned inside a second sheath and advanced along the access path and into the interior. The first sheath and the second sheath may be the same sheath. The first sheath may be different from the second sheath.

The methods may include advancing a cavity preparation instrument along the access path and into the interior. The methods may include positioning the cavity preparation instrument in the interior. The cavity preparation instrument may include one or more features of apparatus and methods described in U.S. patent application Ser. No. 13/009,657, filed on Jan. 19, 2011, now U.S. Pat. No. 8,961,518, and/or U.S. patent application Ser. No. 14/568,301, filed on Dec. 12, 2014, both of which are hereby incorporated by reference herein in their entireties.

The cavity preparation instrument may be positioned in the interior after the augmenting and before the supporting. The cavity preparation instrument may be positioned in the interior before the augmenting and before the supporting. The cavity preparation instrument may be positioned in the interior both before and after the augmenting.

The cavity preparation instrument may enlarge a hole created in the interior by the drill. The cavity preparation instrument may enlarge the hole to form a cavity.

When the cavity preparation instrument is inserted in the interior after the expansion of the cage, the cavity preparation instrument may enlarge a hole created in the interior by expansion of the cage. The cavity preparation may enlarge the hole to create the cavity.

The cavity may be shaped to receive the first cage. The cavity preparation instrument, when expanded, may replicate a shape of the cage that will be implanted in the cavity. The cavity may conform to the size and shape of the first cage. The cavity may be shaped to receive the second cage. The cavity may conform to the size and size and shape of the second cage.

A distal end of the cavity preparation instrument may be disposed, in the interior, at the target site. A distal end of the cavity preparation instrument may be positioned apart from the target site.

The methods may include rotating, in the interior, the cavity preparation instrument. The cavity preparation instrument may include a broaching member. The methods may include radially expanding the broaching member. Expanding the broaching member and rotating the cavity preparation instrument may form a cavity in the interior. The broaching member may be expanded when the cavity preparation instrument is in a rest state. The broaching member may be expanded during rotation of the cavity preparation instrument.

Displacing a broaching member radially outward may be referred to as “activation.” Displacing a broaching member radially inward may be referred to as “deactivation.” Changing positions of broaching member ends relative to each other may cause activation. Changing positions of broaching member ends relative to each other may cause de-activation. The cavity preparation instrument may include an activation mechanism. The mechanism may activate an individual broaching member. The activation mechanism may activate a plurality of broaching members. The mechanism may include a linear broaching member actuator. The mechanism may include a rotating broaching member actuator. The rotating broaching member actuator may convert rotation to translation of a broaching member. The mechanism may include one or more of the features. The mechanism may be operated manually. The mechanism may be mechanically assisted. The mechanism may be motorized. The mechanism may be automated. The mechanism may include one or more of the features. The mechanism may include a spring that assists activation. The mechanism may activate one or more broaching members as the tool is rotated. The mechanism may be robotic. The tool may be robotic.

The methods may include forming the cavity in the interior.

Forming the cavity may be performed after the augmenting and before the supporting. The cavity may have a first volume. The augmenting may include radially expanding the first cage to a second volume. The second volume may be greater than the first volume. The supporting may include expanding the second cage to a third volume. The third volume may be equal to the second volume. The third volume may be greater than the second volume. The third volume may be less than the second volume.

Forming the cavity may be performed before the augmenting and before the supporting. The cavity may have a first volume. The augmenting may include radially expanding the first cage to a second volume. The second volume may be greater than the first volume. The supporting may include expanding the second cage to a third volume. The third volume may be equal to the second volume. The third volume may be greater than the second volume. The third volume may be less than the second volume.

Forming the cavity may be performed both before the augmenting and after the augmenting. The supporting may be performed after the augmenting. The cavity formed before the augmenting may have a first volume. The cavity formed after the augmenting may have a second volume. The second volume may be greater than the first volume. The augmenting may include radially expanding the first cage to a third volume. The second volume may be greater than the third volume. The second volume may be equal to the third volume.

The cavity may have a first volume. The second cage may be radially expanded to the first volume. The second cage may be radially expanded to a second volume. The second volume may be greater than the first volume. The second volume may be less than the first volume.

When the second volume is greater than the first volume, expanding the second mesh cage may augment the height of the spinal body. The expansion of the second cage may deform the second cage under a load applied to the second cage during the expansion. Illustrative deformation of a cage under a load applied during expansion is illustrated in FIGS. 7 and 8.

Forming the cavity may include rotating, in the interior, the cavity preparation instrument. The cavity preparation instrument may include the broaching member. Forming the cavity may include radially expanding the broaching member in the interior.

When the spinal body is an intervertebral disc, forming the cavity may include rotating the broaching member in the interior to scrape a first vertebral endplate disposed between the intervertebral disc and the first vertebra. Forming the cavity may include rotating the broaching member in the interior to scrape a second vertebral endplate disposed between the intervertebral disc and the second vertebra.

The broaching member may include an elongated body. The elongated body may include a cutting edge. A distal end of the elongated body may be wrapped around a distal end of the cavity preparation instrument.

The broaching member may include a laser-cut tube that, when expanded, forms a third mesh cage. The third mesh cage may include one or more cutting edges.

Forming the cavity may not include not using a rongeurs, kerrisons, curettes, shavers or balloons.

After forming the cavity, the broaching member may be collapsed. The cavity preparation instrument may be removed from the interior.

The methods may include irrigating disc material displaced by the broaching member. The methods may include aspirating disc material displaced by the broaching member.

The methods may include not irrigating disc material displaced by the broaching member. The methods may include not aspirating disc material displaced by the broaching member.

The methods may include selecting the second cage. The second cage may be selected from a kit including different sized cages. Selecting the second cage may include inserting a sizer or a trial instrument into the interior to measure the volume of the cavity. The measured dimensions may be used by a practitioner to select an optimally sized cage from the kit. Selecting the second cage may include selecting the second cage based at least in part on the expansion of the cavity preparation instrument.

Augmenting the height of the spinal body may include positioning the broaching member in the interior.

When the spinal body is an intervertebral disc, the augmenting may include expanding the broaching member. The augmenting may include positioning the broaching member against the first vertebra and the second vertebra. The augmenting may include positioning the first mesh cage against the intervertebral disc. The augmenting may include positioning the first mesh cage against the first vertebra and the intervertebral disc.

When the spinal body is a vertebra, the augmenting may include expanding the broaching member. The augmenting may include positioning the broaching member against the vertebra.

The methods may include using a unilateral approach for treating the spinal body. The unilateral approach may include augmenting the height of the spinal body by expanding the first cage. The unilateral approach may include supporting the spinal body by expanding the second cage.

The methods may include using a bilateral approach for treating the spinal body. The bilateral approach may include augmenting the height of the spinal body by expanding the first cage and a third cage in the interior. The bilateral approach may include supporting the spinal body by expanding the second cage and a fourth cage in the interior.

In the bilateral approach, the access hole may be a first access hole. The access path may be a first access path. The probe may be a first probe. The target site may be a second target site.

The methods may include preparing a second access hole on the surface of the patient's body. The methods may include advancing a second probe through the second access hole and into the interior. The second probe, when positioned in the interior, may prepare a second access path. A distal end of the second probe may be positioned at a second target site. A drill may be advanced over second probe and to the second target site. The third cage may be advanced along the second access path and into the interior. A distal end of the third cage may be positioned at the second target site. A distal end of the third cage may be positioned apart from the second target site.

In the bilateral approach, the first access path may define a first angle relative to a sagittal plane. The second access path may be opposite the first access path along the sagittal plane and define a second angle relative to a sagittal plane. The first angle may be substantially equivalent to the second angle.

In the bilateral approach, the augmenting may include expanding the first cage and the third cage in the interior. The third cage may be radially expanded. The third cage may be a third mesh cage. The third mesh cage may be formed from a laser-cut tube. The third mesh cage, when expanded, may form a mesh. The mesh may include a plurality of interconnected cells.

In the bilateral approach, after the first cage and the third cage are expanded in the interior, the methods may include collapsing the first cage and the third cage. The methods may include removing the first cage and the third cage from the interior.

In the bilateral approach, the cavity preparation instrument may be a first cavity preparation instrument and the cavity may be a first cavity. A second cavity preparation instrument may be advanced along the second access path and used to create a second cavity. The first cavity may have a first volume. The second cavity may have a second volume. The first volume may be equivalent to the second volume. The first volume may be larger than the second volume. The first volume may be smaller than the second volume.

In the bilateral approach, the supporting may include expanding the second cage and the fourth cage in the interior. The fourth mesh cage may be advanced through the second access hole, along the second access path and into the interior. A distal end of the fourth cage may be positioned at the second target site. The distal end of the fourth cage may be positioned apart from the second target site.

The fourth cage may be a fourth mesh cage. The fourth mesh cage may be formed from a laser-cut tube. The laser-cut tube, when expanded, may form a mesh. The mesh may include a plurality of interconnected cells. The fourth mesh cage may be radially expanded in the interior.

The fourth cage may be expanded to a first volume. The second cage may be expanded to a second volume. The first volume may be equivalent to the second volume. The first volume may be smaller than the second volume. The first volume may be larger than the second volume. The first volume may be equal to the volume of the second cavity. The first volume may be different from the volume of the second cavity.

In the bilateral approach, the second cage and the fourth cage may be surgically enclosed in the interior.

In the bilateral approach, the first mesh cage may be positioned in the interior at a right lateral location. The third mesh cage may be positioned in the interior at a left lateral location. The second mesh cage may be positioned in the interior at a right lateral location. The fourth mesh cage may be positioned in the interior at a left lateral location.

In the bilateral approach, the first, second, third and fourth mesh cages may define, respectively, first, second third and fourth central axes. The first and third mesh cages may be positioned in the interior so that the first central axis is convergent with the third central axis in a transverse plane. The first mesh cage may be positioned apart from the third mesh cage in the interior. The first mesh cage may abut the third mesh cage. The second and fourth mesh cages may be positioned in the interior so that the second central axis is convergent with the fourth central axis in the transverse plane. The second mesh cage may be positioned apart from the fourth mesh cage in the interior. The second mesh cage may abut the fourth mesh cage.

In the bilateral approach, methods disclosed using the first cage may be performed with the third cage. In the bilateral approach, methods disclosed using the second cage may be performed with the fourth cage.

The methods may include seating the second mesh cage in the interior. The second mesh cage may be seated in the interior after expanding the second mesh cage in the interior. The second mesh cage may be expanded to a first volume.

The second mesh cage may be seated in the interior by rotating the second mesh cage. The second mesh cage may define a central axis. The second mesh cage may be rotated about the central axis. The rotating may include depositing displaced bone in an interior of the second mesh cage. The displaced bone may be bone that was displaced by the cavity preparation instrument.

The second mesh cage may expand elastically. The second mesh cage may expand elastically to a second volume greater than the first volume. The second mesh cage may expand elastically during the rotation. The second mesh cage may expand radially. The radial expansion of the second mesh cage in the interior may augment the height of the spinal body.

The first cage may be a double-layered cage. Radially expanding the first mesh cage may include expanding a first tube. The first tube may be radially expanded. The first tube may be a laser-cut tube. The first laser-cut tube, when expanded, may form a first mesh defining a plurality of interconnected cells. The first laser-cut tube, when radially expanded, may form a first shape. Radially expanding the first mesh cage may include expanding a second tube. The second tube may be radially expanded. The second tube may be a laser-cut tube. The second laser-cut tube, when radially expanded, may form a second mesh defining a plurality of interconnected cells. The second laser-cut tube, when radially expanded, may form a second shape. The second shape may be formed within the first shape. The second tube, in the second shape, may buttress the first tube.

The second cage may be a double-layered cage. Radially expanding the second mesh cage may include expanding the first tube and the second tube.

The first cage may be a triple-layered. Radially expanding the first mesh cage may include expanding the first laser-cut tube. Radially expanding the first mesh cage may include expanding the second laser-cut tube. Radially expanding the first mesh cage may include expanding a third tube. The third tube may be radially expanded. The third tube may be laser-cut. The third laser-cut tube, when radially expanded, may form a third mesh defining a plurality of interconnected cells. The third laser-cut tube, when radially expanded, may form a third shape. The third shape may be formed within the second shape. The third tube, in the third shape, may buttress the first and second tubes.

The second cage may be a triple-layered cage. Radially expanding the second mesh cage may include expanding the first tube, the second tube and the third tube.

The first cage and the second cage may be double-layered cages. Radially expanding the first mesh cage may include radially expanding the first tube and the second tube. Radially expanding the second mesh cage may include radially expanding a third tube. The third tube may be laser-cut. The third tube may be radially expanded. The third tube, when radially expanded, may form a third mesh defining a plurality of interconnected cells. The third laser-cut tube, when radially expanded, may form a third shape. Radially expanding the second mesh cage may include expanding a fourth tube. The fourth tube may be radially expanded. The fourth tube may be laser-cut. The fourth tube, when radially expanded, may form a fourth mesh defining a plurality of interconnected cells. The fourth laser-cut tube, when radially expanded, may form a fourth shape. The fourth shape may be formed within the third shape. The fourth tube, in the fourth shape, may buttress the third tube.

The first cage may be a double-layered cage and the second cage may be a triple-layered cage. Radially expanding the first mesh cage may include radially expanding the first tube, the second tube and the third tube. Radially expanding the second mesh cage may include radially expanding a fourth tube. The fourth tube may be laser-cut. The fourth tube may be radially expanded. The fourth tube, when radially expanded, may form a fourth mesh defining a plurality of interconnected cells. The fourth laser-cut tube, when radially expanded, may form a fourth shape. Radially expanding the second mesh cage may include expanding a fifth tube. The fifth tube may be radially expanded. The fifth tube may be laser-cut. The fifth tube, when radially expanded, may form a fifth mesh defining a plurality of interconnected cells. The fifth laser-cut tube, when radially expanded, may form a fifth shape. The fifth shape may be formed within the fourth shape. The fifth tube, in the fifth shape, may buttress the fourth tube.

The methods may include filling an interior of the second cage with bone cement. The methods may include filling an interior of the second cage with bone graft.

The second cage may be expanded without using a balloon. The second cage may be filled with bone cement without using a balloon.

The methods may include not advancing material through a central member extending along a central axis of the second cage and into an interior of the second cage. The material may include bone graft. The material may include bone cement. The bone cement may be any suitable bone cement, such as Ploymethyl Methacrylate (“PMMA”). The methods may include not using a balloon.

The methods may include, after surgically enclosing the second cage in the interior, accessing the interior. The methods may include collapsing the second mesh cage. The methods may include removing the second mesh cage from the interior.

The supporting may include positioning a plate on the spinal body.

When the spinal body is an intervertebral disc, the supporting may include advancing a screw through the first vertebra and into the second mesh cage. When a plate is positioned on the first vertebra, the methods may include advancing the screw through the plate. The screw may be advanced through the first cell. The screw may be advanced through the second cell. The screw may be advanced into the second vertebra.

The screw may be a pedicle screw or any other suitable screw known to those skilled in the art. The screw may not be a pedicle screw.

When the spinal body is an intervertebral disc, the supporting may include driving a fixation element through the first vertebra and into the second mesh cage. The fixation element may be a k-wire or a probe. When a plate is positioned on the first vertebra, the methods may include advancing the fixation element through the plate. The fixation element may be advanced through the first cell. The fixation element may be advanced through the second cell. The fixation element may be advanced into the second vertebra. A cannulated screw may be advanced along the fixation element, through the first vertebra, and through the first cell. The cannulated screw may be advanced through the second cell. The cannulated screw may be advanced through the plate. The cannulated screw may be advanced into the second vertebra.

When the spinal body is an intervertebral disc, the supporting may include positioning a plate on the first vertebra. The supporting may include positioning the plate on the second vertebra. The plate may extend between the first vertebra and the second vertebra.

When the spinal body is an intervertebral disc, the methods may include not placing bone graft on external fixation mechanisms fixed to the first vertebra. The external fixation mechanisms may include one or more screws. The external fixation mechanisms may include one or more rods. A rod may extend between a first screw fixed to the first vertebra and a second screw fixed to the second vertebra. The external fixation mechanisms may include one or more plates.

When the spinal body is an intervertebral disc, the methods may include placing bone graft on the external fixation mechanisms.

When the spinal body is an intervertebral disc, the supporting may include advancing a screw through the first vertebra, through the second mesh cage, and into the second vertebra. The screw may be advanced through the first cell. The screw may be advanced through the second cell.

When the spinal body is an intervertebral disc, the supporting may include driving a fixation element through the first vertebra, through the second mesh cage and into the second vertebra. When a plate is positioned on the first vertebra, the fixation element may pass through the plate. The fixation element may pass through the first cell and the second cell. A cannulated screw may be advanced along the fixation element, through the first vertebra, the first cell and the second cell, and into the second vertebra. When a plate is positioned on the first vertebra, the cannulated screw may pass through the plate.

When the spinal body is an intervertebral disc, the supporting may include inserting a screw into the first vertebra and expanding a distal end of the screw to form a third cage. The third cage may be a third mesh cage. The third mesh cage, when expanded, may form a mesh. The mesh may include a plurality of interconnected cells. The third mesh cage may be formed from a laser-cut tube.

When the spinal body is an intervertebral disc, the supporting may include inserting a first screw into the first vertebra and expanding a distal end of the first screw to form the third cage. The supporting may include inserting a second screw into the second vertebra and expanding a distal end of the second screw to form a fourth cage. The fourth cage may be a fourth mesh cage. The fourth mesh cage, when expanded, may form a mesh. The mesh may include a plurality of interconnected cells. The fourth mesh cage may be formed from a laser-cut tube.

When the spinal body is an intervertebral disc, the methods may include accessing the interior through an anterior side of the intervertebral disc. The methods may include accessing the interior through a posterior side of the intervertebral disc. The methods may include accessing the interior through a lateral side of the intervertebral disc. The methods may include accessing the interior through a posterolateral angle defined by a posterior side and a lateral side of the intervertebral disc. When the first vertebra is a cervical vertebra, the methods may include accessing the interior through an anterior face of the intervertebral disc.

When the spinal body is a vertebra, the supporting may include positioning a plate on the vertebra. The supporting may not include positioning a plate on the vertebra.

When the spinal body is a vertebra, the supporting may include advancing a screw through the vertebra and into the second mesh cage. The screw may be advanced through the first cell. The screw may be advanced through the second cell. The screw may be advanced through both the first cell and the second cell. When a plate is positioned on the vertebra, the screw may be advanced through the plate.

When the spinal body is a vertebra, the supporting may include driving a fixation element through the vertebra and through the first cell. The fixation element may be advanced through the second cell. When a plate is positioned on the vertebra, the fixation element may be advanced through the plate. A cannulated screw may be advanced over the fixation element, through the vertebra and through the first cell. When a plate is positioned on the vertebra, the cannulated screw may be advanced through the plate. The cannulated screw may be advanced through the second cell.

When the spinal body is a vertebra, the methods may include accessing the interior through an anterior side of the vertebra. The methods may include accessing the interior through a posterior side of the vertebra. The methods may include accessing the interior through a lateral side of the vertebra. The methods may include accessing the interior through a posterolateral angle defined by a posterior side and a lateral side of the vertebra. When the first vertebra is a cervical vertebra, the methods may include accessing the interior through an anterior face of the vertebra.

When the spinal body is a vertebra, the methods may include not placing bone graft on the external fixation mechanisms fixed to the vertebra.

When the spinal body is a vertebra, the methods may include placing bone graft on the external fixation mechanisms.

When the spinal body is a vertebra, the methods may include inserting a screw into the vertebra and expanding a distal end of the screw to form a third cage. The third cage may be a third mesh cage. The third mesh cage, when expanded, may form a mesh. The mesh may include a plurality of interconnected cells. The third mesh cage may be formed from a laser-cut tube.

When the spinal body is a vertebra, the vertebra may be a cervical vertebra. The vertebra may be a thoracic vertebra. The vertebra may be a lumbar vertebra. The vertebra may be a sacral vertebra.

The vertebra may be accessed using a transpedicular approach. A transpedicular approach may accesses the vertebra through the pedicle and then into the vertebral body. The vertebra may be accessed using an extrapedicular approach. An extrapedicular approach may directly access the vertebra directly without going through the pedicle.

When the spinal body is an intervertebral disc, the first vertebra may be a cervical vertebra. The first vertebra may be a thoracic vertebra. The first vertebra may be a lumbar vertebra. The first vertebra may be a sacral vertebra. The second vertebra may be a cervical vertebra. The second vertebra may be a thoracic vertebra. The second vertebra may be a lumbar vertebra. The second vertebra may be a sacral vertebra.

The methods may be applied to a spinal body during a procedure. The methods may be applied to two, three, four or more spinal bodies during a procedure. The two, three, four or more spinal bodies may be adjacent. One of the spinal bodies may be not be adjacent the other spinal bodies.

The methods may be performed through a single access hole. The methods may be performed along a single access path.

The methods may be performed through two, three, four or more access holes. The methods may be performed along two, three, four or more access paths.

The methods, which may involve the apparatus shown and described herein, may include methods for treating a spinal body having an interior. The spinal body may be a vertebra. The spinal body may be an intervertebral disc. The methods may include one or more steps from any other methods disclosed herein.

The methods may include augmenting a height of the spinal body. The augmenting may include radially expanding the first cage in the interior.

Radially expanding the first cage may include expanding a first tube. The first tube may be radially expanded. The first tube may be formed from laser-cut tube. The first tube, when radially expanded, may form a first mesh including a plurality of interconnected cells. The first laser-cut tube, when radially expanded, may form a first shape. Radially expanding the first mesh cage may include expanding a second tube. The second tube may be radially expanded. The second tube may be formed from laser-cut tube. The second tube, when radially expanded, may form a second mesh including a plurality of interconnected cells. The second laser-cut tube, when radially expanded, may form a second shape. The second shape may be formed within the first shape. The second tube, in the second shape, may buttress the first tube.

Radially expanding the first cage may include expanding the first laser-cut tube. Radially expanding the first mesh cage may include expanding the second laser-cut tube. Radially expanding the first mesh cage may include expanding a third tube. The third tube may be radially expanded. The third tube may be formed from laser-cut tube. The third tube, when radially expanded, may form a third mesh including a plurality of interconnected cells. The third laser-cut tube, when expanded, may form a third shape. The third shape may be formed within the second shape. The third tube, in the third shape, may buttress the first and second tubes.

The methods may include removing the first mesh cage from the interior.

The methods may include, after removing the first mesh cage from the interior, supporting the spinal body in an elevated position by filling the interior with bone cement. The portion of the interior filled with bone cement may be a portion of the interior occupied by the expanded first mesh cage. The methods may include surgically enclosing the bone cement in the cavity. Filling the cavity with bone cement may not include using a balloon.

The methods may include forming a cavity in the interior. The cavity may be formed after removing the first mesh cage from the interior. The cavity may be formed after removing the first cage from the interior. Forming the cavity may include rotating the cavity preparation instrument in the interior. The cavity preparation instrument may include the broaching member. Forming the cavity may include expanding the broaching member in the interior. The broaching member may be radially expanded in the interior.

The methods may include supporting the spinal body in an elevated position by filling the cavity with bone cement. The spinal body may be supported after forming the cavity with the broaching member. The methods may include surgically enclosing the bone cement in the cavity. Filling the cavity with bone cement may not include using a balloon.

The methods may include, after removing the first cage from the interior, supporting the spinal body in an elevated position by expanding the second mesh cage in the cavity. The second mesh cage may be radially expanded. The methods may include surgically enclosing the second cage in the interior. The methods may include surgically enclosing the first mesh cage in the interior. Prior to surgically enclosing the first mesh cage, the methods may include supporting the spinal body in an elevated position. The supporting may include any of the supporting methods described herein.

The methods, which may involve the apparatus shown and described herein, may include methods for treating a spinal body having an interior. The spinal body may be a vertebra. The spinal body may be an intervertebral disc. The methods may include one or more steps from any other methods disclosed herein.

The methods may include forming a cavity in the interior. The cavity may be formed by rotating in the interior the cavity preparation instrument. The cavity preparation instrument may include the broaching member. The broaching member may be expanded in the interior. The broaching member may be radially expanded in the interior.

The methods may include, after forming the cavity, filling the cavity with material. The material may be bone cement. The methods may include supporting the spinal body using one or more of the external fixation mechanisms. The methods may include surgically enclosing the bone cement in the interior.

The methods may include, after forming the cavity, radially expanding the cage in the cavity. The cage may be a mesh cage. The cage may be the first mesh cage. The mesh cage, when expanded, may form a plurality of interconnected cells.

Expanding the cage may augment a height of the spinal body. The augmenting may include expanding the first cage in the interior. The cage may be expanded to a first volume. The cavity may have a second volume. The first volume may be greater than the second volume.

Radially expanding the mesh cage may include expanding a first tube. The first tube may be radially expanded. The first tube may be laser-cut. The first tube, when radially expanded, may form a first mesh defining a plurality of interconnected cells. The first tube, when radially expanded, may form a first shape. Radially expanding the mesh cage may include expanding a second tube. The second tube may be radially expanded. The second tube may be laser-cut. The second tube, when radially expanded, may form a second mesh defining a plurality of interconnected cells. The second tube, when radially expanded, may form a second shape. The second shape may be formed within the first shape. The second tube, in the second shape, may buttress the first tube.

Radially expanding the mesh cage may include expanding the first tube. Radially expanding the mesh cage may include expanding the second tube. Radially expanding the mesh cage may include expanding a third tube. The third tube may be radially expanded. The third tube may be laser-cut. The third tube, when expanded, may form a mesh defining a plurality of interconnected cells. The third tube, when expanded, may form a third shape. The third shape may be formed within the second shape. The third tube, in the third shape, may buttress the first and second tubes.

The methods may include filling an interior of the mesh cage with material. The filling may be performed after expanding the cage. The material may be bone cement. The material may be bone graft. The methods may include surgically enclosing the cage in the interior after filling the interior with material. The methods may include filling the interior of the mesh cage with material without using a balloon.

The methods may include not advancing the material through a central member extending along a central axis of the mesh cage and into an interior of the mesh cage.

The methods may include surgically enclosing the cage in the interior. The cage may be surgically enclosed in the interior after radially expanding the cage.

The cage may include a first cell. The cage may include a second cell.

When the spinal body is a vertebra, and prior to surgically enclosing the cage in the interior, the methods may include advancing a screw through the vertebra and into the first cell. The methods may include advancing the screw through the second cell. The methods may include supporting the vertebra using one or more of the fixation mechanisms. The methods may include placing a plate on the vertebra. The screw may be advanced through the plate.

When the spinal body is a vertebra, the methods may include advancing the fixation element through the vertebra and into the first cell. The methods may include advancing the fixation element into the second cell. The methods may include advancing a cannulated screw along the fixation element and through the first cell. The cannulated screw may be advanced through the second cell. The methods may include placing a plate on the vertebra. The fixation element and the cannulated screw may be advanced through the plate.

The methods may include methods for positioning a first bone relative to a second bone, the first bone having a first interior and the second bone having a second interior. The methods may include positioning a first cage in the first interior; positioning a second cage in the second interior; radially expanding the first cage to form a first mesh cage and radially expanding the second cage to form a second mesh cage; reducing a distance between the first bone and the second bone by shortening a distance between the first cage and the second cage; and surgically enclosing the first cage and the second cage in the interiors.

The first cage may have one or more features in common with the cage. The second cage may have one or more features in common with the cage.

The first bone may be an ilium. The second bone may be a sacrum.

Radially expanding the first cage may include urging the first cage out the end of a delivery sheath so that the first cage self-expands. Radially expanding the second cage may include urging the first cage out the end of a delivery sheath so that the second cage self-expands. Radially expanding the first cage may include rotating a threaded actuator member to draw opposite ends of the first cage toward each other. Radially expanding the second cage may include rotating a threaded actuator member to draw opposite ends of the second cage toward each other.

The reducing may include rotating a central threaded member relative to two end members, each threaded opposite the other, and each supporting one of the cages. The reducing may include rotating one threaded member relative to another threaded member that is threadingly engaged to the one threaded member.

The methods may include methods for securing a spinal body. The spinal body may be a vertebra. The spinal body may be an intervertebral disc.

The methods may include turning a screw that is engaged with and traverses a wall of the spinal body to advance a mesh cage to a position interior the vertebra. When the spinal body is a vertebra, the wall may be a vertebral wall. When the spinal body is an intervertebral disc, the wall may be a wall of an intervertebral disc.

The screw may define a longitudinal direction. The methods may include drawing together longitudinally a distal end of the mesh cage and a proximal end of the mesh cage to expand the mesh cage away from the longitudinal axis.

The mesh cage may have one or more features in common with the first cage. The mesh cage may have one or more features in common with the second cage.

The methods may be used during a kyphoplasty procedure. The methods may be used during a vertebroplasty procedure. The methods may be used as part of an intervertebral disc replacement. The methods may be used to restore kyphosis. The methods may be used to treat osteoporotic or poor bone quality. The methods may be used for any other conditions described herein.

The spinal body may have a compromised shape. The spinal body may have a non-compromised shape.

The screw may be a first screw. The drawing may include turning a second screw to reduce a distance between the distal end and the proximal end.

The turning of the second screw may include inserting a driver through a cannula in the first screw; and engaging the driver with the second screw.

The drawing may include hingedly articulating the distal end relative to an atraumatic tip and the proximal end relative to the first screw.

The methods may include bracing the screw, exterior the spinal body, to a pedicle screw that is engaged to a different spinal body.

The methods may include filling the expanded mesh cage with material such as bone cement or morcellated bone.

The methods may include augmenting a height of the spinal body by expanding the mesh cage in the interior. Augmenting the height of the spinal body may lift and/or decompress the spinal body. The methods may include supporting the spinal body in an elevated position by implanting the mesh cage in the interior.

The screw may be advanced into the spinal body prior to preparing a cavity in the interior using the cavity preparation instrument. The screw may be advanced into the spinal body after a cavity has been prepared in the interior using the cavity preparation instrument.

The screw may be advanced into the spinal body after a height of the spinal body has been augmented using one or more of a spacer, an expanded cage, patient positioning or a surgical table. Expanding the mesh cage may support the spinal body in an elevated position.

The screw may be advanced into the patient's body to the spinal body using any suitable percutaneous or open screw placement techniques known to those skilled in the art. For example, a guide wire or a tap may be used.

The methods may include methods for securing a spinal body. The spinal body may be a vertebra. The spinal body may be an intervertebral disc.

The methods may include expanding a mesh cage inside the spinal body, the mesh cage defining a longitudinal axis; with a threaded member moving along the longitudinal axis toward a distal end of the mesh cage, deflecting away from the longitudinal axis, an extension that extends from a distal end of the mesh cage; and driving the threaded member into a region of the spinal body distal the mesh cage.

The mesh cage may have one or more features in common with the first spinal body. The mesh cage may have one or more features in common with the second spinal body.

The methods may include providing, through the threaded member, to a space between the threaded member and an interior surface of the mesh cage a therapeutic material.

The providing may include flowing the material through a cannula coaxial with the threaded member. The providing may include flowing the material through a cannula that is oblique to a longitudinal axis of the threaded member. The providing may include flowing the material through a cannula that is oblique to the longitudinal axis.

The methods may include preparing for the mesh cage a cavity in the spinal body. The methods may include releasing the mesh cage in the cavity. The methods may include threadingly advancing the threaded member through a bushing at a proximal end of the spinal body. The mesh cage may self-expand from a contracted state.

The threadingly advancing may include causing the threaded member to traverse a pedicle wall.

The methods may include methods for treating a spinal body. The spinal body may be a vertebra. The spinal body may be an intervertebral disc.

The methods may include preparing a cavity in the spinal body. The cavity may have a spinal body concavity. The methods may include expanding in the cavity a mesh cage. The mesh cage may have a cage concavity. The cage concavity may conform to the first concavity.

The mesh cage may have one or more features in common with the first cage. The methods may have one or more features in common with the second cage.

The spinal body cavity may be a first spinal body concavity. The cage concavity may be a first cage concavity.

The methods may include preparing a second spinal body concavity in the spinal body. The methods may include expanding in the cavity a second mesh cage concavity. The second mesh cage concavity may conform to the second spinal body concavity.

The methods may include positioning the cage. The first cage may be positioned so that the first cage concavity faces posterior. The first cage may be positioned so that the second cage concavity faces anterior.

The apparatus may include, and the methods may involve, apparatus for securing a spinal body. The spinal body may be a vertebra. The spinal body may be an intervertebral disc.

The apparatus may include a cannulated tapered tip having an atraumatic outer surface. The apparatus may include a cannulated shaft having tissue-engaging threads. The shaft may be collinear with, and longitudinally spaced apart from the tip. The apparatus may include a mesh member that extends longitudinally from the tip to the shaft. The mesh member may expand radially outward in response to being compressed between the tip and the shaft

The mesh member may have one or more features in common with the first cage. The mesh member may have one or more features in common with the second cage.

The mesh in a contracted state may be a mesh that has a diameter that is no greater than a diameter of the shaft. The mesh in a contracted state may have a diameter that is: greater than a diameter of the shaft; and no greater than a diameter defined by a threading on the shaft. The mesh in a contracted state may have a diameter that is greater than a diameter defined by a threading on the shaft. The mesh in an expanded state may have a diameter that is no greater than a diameter of the shaft.

The mesh in an expanded state may have a diameter that is: greater than a diameter of the shaft; and no greater than a diameter defined by a threading on the shaft.

The mesh in an expanded state may have a diameter that is greater than a diameter defined by a threading on the shaft

The apparatus may include a fin, the mesh supporting the fin, the fin extending radially away from the mesh. The fin may extend transverse to the longitudinal direction of the screw.

The apparatus may include a tulip-head engaged to the shaft.

The actuator may include a cannulated threaded rod having: a distal end and a proximal end. The distal end may threadingly engage the tip; the shaft may restrain longitudinally the proximal end; and the proximal end may be configured to receive through the cannulated shaft a driver to turn the threaded rod and thereby change the distance.

The apparatus may include, and the methods may involve, apparatus for securing a spinal body. The spinal body may be a vertebra. The spinal body may be an intervertebral disc.

The apparatus may include a cannulated tapered tip having an atraumatic outer surface and first internal threading. The apparatus may include a cannulated shaft having tissue-engaging threads. The shaft may be collinear with, and longitudinally spaced apart from the tip. The apparatus may include a mesh member that extends longitudinally from the tip to the shaft, and expands radially outward in response to elastic energy stored in the mesh. The apparatus may include second internal threads fixed to the shaft. The first and second internal threads may be collinear and spaced apart longitudinally from each other by a distance corresponding to an extent of expansion of the mesh.

The apparatus may include a cannulated threaded rod that is threadingly engaged with the first internal threads and the second internal threads to retain the mesh in a contracted state.

The apparatus may include, and the methods may involve, apparatus for securing a spinal body. The apparatus may include: a cannulated screw having a tulip head; a bushing that is internally threaded to threadingly engage the screw. The apparatus may include an expandable mesh cage. The mesh cage may have one or more features in common with the first mesh cage. The mesh cage may have one or more features in common with the second cage.

A first end of the mesh cage may be fixed to the bushing; and a second end of the mesh cage may include an extension that is biased toward, and reach to within a zone of, the longitudinal axis, the zone having a diameter that is lesser than a major diameter of the screw.

The cannulated screw may define a window that traverses a root of the screw.

The cage may be hinged to the bushing.

The extension may have an end adjacent the longitudinal axis.

The extension may be a first extension; the first extension may have a distal end adjacent the longitudinal axis. The link may extend from the distal end to a distal end of a second extension, the link operative to counteract spreading between the first extension and the second extension when the screw advances by the extensions.

The apparatus may include, and the methods may involve, apparatus for reducing a gap between first body tissue and second body tissue. The apparatus may include: a plate; an expandable mesh cage; and first and second threadingly engageable members that define a longitudinal direction and form an adjustable-length shaft that changes a longitudinal distance between the plate and the cage. The plate: may extend in a radial direction away from the longitudinal axis and, in the radial direction, terminate at a smooth surface; and may be of a construction that is monolithic with the first threadingly engageable member.

The surface may be a surface that does not have threading. The surface may be a surface that does not include a sharp edge. The surface may be configured to not cut bone. The surface may be uniformly and entirely smooth.

The apparatus may include a circumferential hinge by which the second threadingly engageable member is attached to the expandable mesh cage.

The hinge may include circumferentially distributed T-slots that retain roofs of corresponding T-tabs that extend from the mesh, the slots providing clearance for stems of the T-tabs to angulate relative to the longitudinal axis when the mesh changes shape.

The apparatus may include, and the methods may involve, apparatus for reducing a gap between a first body tissue and a second body tissue. The apparatus may include an actuator assembly that has a first end and a second end a distance away from the first end; extending from the first end, a distal expandable cage; and, extending from the second end, a proximal expandable cage; the actuator assembly is configured to reduce the distance.

The distal expandable cage may have one or more features in common with the first cage. The distal expandable cage may have one or more features in common with the second cage. The proximal expandable cage may have one or more features in common with the first cage. The proximal expandable cage may have one or more features in common with the second cage.

The first end may include a first threading. The second end may include a second threading that is engaged with the first threading. The distance may be reduced by increasing the extent to which the first and second threadings are threadingly engaged.

The first end may include a first threading; the second end may include a second threading having thread direction opposite that of the first threading; and an intermediate actuator assembly component disposed between the first end and the second end may include a third threading engaged with the first threading, and a fourth threading engaged with the second threading. The distance may be reduced by causing relative rotation between (a) the intermediate member; and (b) the first end and the second end.

The first end may include a first sleeve. The second end may include a second sleeve that extends into the first sleeve. The distance may be reduced by non-rotatingly drawing the second sleeve into the first sleeve.

One of the first and second sleeves may include a catch and the other may include a protrusion, the catch and the protrusion being engageable by causing relative rotation between the first and second sleeve to bring the catch and the protrusion into engagement after the distance is reduced.

One of the first and second sleeves may include a catch, and the other may include a protrusion, the catch and the protrusion being disengageable by causing relative rotation between the first and second sleeve to release the catch from the protrusion before the distance is reduced.

The first end may include a bracket for rotatably retaining a head of a distal cage actuator screw, the screw engaged with threading at a distal end of the distal cage, turning of the distal cage actuator screw shortening and expanding the distal cage.

The second end may include a bushing having internal threading for engagement by a proximal actuator screw that is rotatably held at the proximal end of the proximal cage, turning of the proximal cage actuator screw shortening and expanding the proximal cage.

The proximal cage actuator screw may be cannulated. The cannulation may provide access by a driver to the head. The driver may be used to apply torque to the head.

Apparatus and methods described herein are illustrative. Apparatus and methods in accordance with the invention will now be described in connection with the FIGs, which form a part hereof. The FIGS. show illustrative features of apparatus and method steps in accordance with the principles of the invention. It is to be understood that other embodiments may be utilized and that structural, functional and procedural modifications may be made without departing from the scope and spirit of the present invention.

The steps of the methods may be performed in an order other than the order shown and/or described herein. Embodiments may omit steps shown and/or described in connection with the illustrative methods. Embodiments may include steps that are neither shown nor described in connection with the illustrative methods. Illustrative method steps may be combined. For example, one illustrative method may include steps shown in connection with another illustrative method.

Some apparatus may omit features shown and/or described in connection with illustrative apparatus. Embodiments may include features that are neither shown nor described in connection with the illustrative methods. Features of illustrative apparatus may be combined. For example, one illustrative embodiment may include features shown in connection with another illustrative embodiment.

The apparatus and methods of the invention will be described in connection with embodiments and features of illustrative devices. The devices will be described now with reference to the accompanying drawings in the FIGS., which form a part hereof.

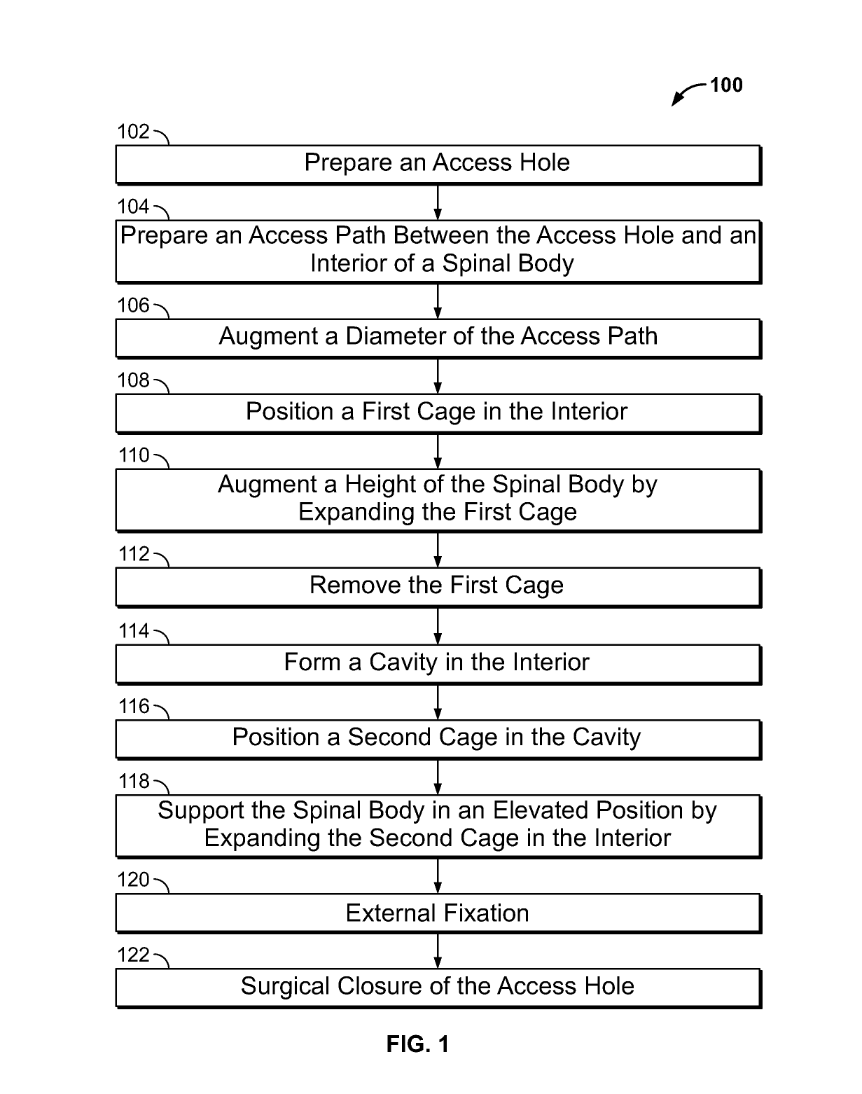

FIG. 1 shows illustrative method 100 in accordance with the invention. Method 100 may be a method for treating a spinal body having an interior. The spinal body may be a vertebra. The spinal body may be an intervertebral disc.

The steps included in method 100 may be performed in an order different from the order illustrated in FIG. 1. The steps may be performed using apparatus disclosed herein.

Method 100 may be applied to a unilateral procedure providing therapy to the spinal body. Method 100 may be applied to a bi-lateral procedure providing therapy to the spinal body. The bi-lateral procedure may provide therapy to a first portion of the spinal body and a second portion of the spinal body. The therapy may include applying method 100 to the first portion. The therapy may include applying method 100 to the second portion.

Method 100 may be applied to a single spinal body during a procedure. Method 100 may be applied to two or more spinal bodies during the procedure.

Method 100 may be applied to a vertebra during a procedure. Method 100 may be applied to a vertebra during an interbody disc. Method 100 may be applied to both a vertebra and an interbody disc during a procedure.

At step 102 method 100 may include preparing an access hole. The access hole may be prepared on a surface of the patient's body.

The access hole may be prepared on an anterior side of the body. The access hole may be prepared on a posterior side of the body. The access hole may be prepared on a lateral side of the body. The access hole may be prepared on a posterolateral face of the body. The access hole may be prepared at any other suitable location on the body.

At step 104 method 100 may include defining an access path between the access hole and an interior of a spinal body. Defining the access path may include accessing the interior.

The access path may be prepared by advancing the guide wire through the access hole and into the interior. A tip of the guide wire may be positioned at a target site in the interior.

The access path may be straight. The access path may be curved. The access path may include one or more straight sections and one or more curved sections.

The access path may be prepared using a hand retractor. The access path may be prepared using a retractor instrument. The access path may be prepared using one or more tube dilators.

Accessing the interior of the spinal body may include advancing the guide wire into the interior. Accessing the interior may include visually identifying a position on the spinal body for accessing the interior.

When the spinal body is an intervertebral disc, accessing the interior may include cutting the annulus. The cut portion of the annulus may be removed from the access path. Additional disc material may be removed using a rongeur, kerrison, and/or a curette.

Defining the access path may include using fluoroscopy to confirm a portion of the spinal body. Fluoroscopy may be used to confirm a trajectory of the access path.

At step 106 method 100 may include augmenting the size of the access path.

The augmenting may include drilling along the guide wire. The augmenting may include expanding a retractor advanced along the guide wire. The augmenting may include sliding one or more dilation tubes along the guide wire. The augmenting may include stacking tube retractors over the guide over.

Drilling along the guide wire may be performed manually. Drilling along the guide wire may be performed using a mechanically assisted mechanism. Drilling along the guide wire may include using a motorized mechanism. Drilling along the guide wire may be automated.

A diameter of the drill may be equal to a diameter of the cavity preparation instrument. Drilling may include preparing a path having a maximum diameter equal to a maximum diameter of the first cage. Drilling may include preparing a path having a maximum diameter equal to a maximum diameter of the second cage.

Augmenting the size of the access path may include manually dissecting the spinal body.

After completion of the drilling, the drill may be removed from the interior (step not shown). After completion of the drilling, the guide wire may be removed from the interior (step not shown).

At step 108 method 100 may include positioning a first cage in the interior. The first cage may be advanced into the interior through a tube. The first cage may be advanced into the interior through a retractor.

At step 110 method 100 may include augmenting a height of the spinal body by expanding the first cage. Augmenting the height may decompress impingement of a nerve extending along a face of the spinal body.

The first cage may be expanded incrementally. The first cage may be expanded until a desired augmentation of the height of the spinal body is achieved. Fluoroscopy may be used to verify the elevation.

Expanding the first cage may elevate a fracture of the spinal body. When the spinal body is an intervertebral disc, expanding the first cage may augment a height of the intervertebral disc. When the spinal body is a vertebra, expanding the first cage may augment a height of the vertebra.

The height may be augmented using patient positioning. The height may be augmented using a surgical table.

At step 112 method 100 may include removing the first cage from the interior. The first cage may be collapsed prior to removal. The first cage may be removed from the interior after the height of the spinal body is augmented to a desired elevation.
When the first cage is removed, displaced bone trapped in the cage may be removed together with the cage.

At step 114 method 100 may include forming a cavity in the interior. The cavity may be formed using the cavity preparation instrument.

Forming the cavity may include activating the broaching member. Forming the cavity may include rotating the cavity preparation instrument. When the spinal body is an intervertebral disc, forming the cavity may include scraping one or both vertebra endplates. Scraping vertebra endplate(s) may promote bleeding bone.

Forming the cavity may include using fluoroscopy. Fluoroscopy may be used to verify a volume of the cavity.

The method may include not expanding the broaching member to a diameter greater than a maximum diameter of the second cage, when expanded.

The cavity preparation instrument may be used to augment the height of the spinal body. The cavity preparation instrument may be used to augment the height during preparation of the cavity. The cavity preparation instrument may be used to augment the height after preparation of the cavity.

When the spinal body is an intervertebral disc, forming the cavity may include irrigating displaced disc material. When the spinal body is a vertebra, forming the cavity may include irrigating displaced bone.

When the spinal body is an intervertebral disc, forming the cavity may include manual dissection of disc material. When the spinal body is a vertebra, forming the cavity may include manual dissection of bone.

At step 116 method 100 may include positioning a second cage in the interior. The second cage may be advanced into the interior through a tube. The second cage may be advanced into the interior through a retractor.

At step 118 method 100 may include supporting the spinal body in an elevated position by expanding the second cage in the interior.

The second cage may be expanded to a first volume. The cavity formed at step 114 may have a second volume. The first volume may be equal to the second volume. The first volume may be greater than the second volume. Expanding the second cage to a volume greater than the volume of the cavity may augment the height of the spinal body.

After expanding the second cage to a desired volume, material may be advanced along the access path and into the second cage. Material may be advanced along a central member of the second cage and into the second cage. The material may include bone cement. The material may include bone graft.

Material may not be advanced along the central member and into the second cage.

At step 120 method 100 may include external fixation. External fixation may support the spinal body. The spinal body may be supported in an elevated position.

When the spinal body is a vertebra, external fixation may include driving one or more screws into the vertebra. External fixation may include driving one or more screws into or one or more adjacent vertebras. External fixation may include placing a rod on two adjacent screws.

External fixation may include placing a plate on the vertebra. The plate may be fixed to the vertebra by driving or more screws through the plate and into the vertebra. The plate may be plated on a posterior face of the vertebra. The plate may be plated on an anterior side of the vertebra. The plate may be placed on a lateral face of the vertebra. The plate may be placed on a posterolateral face of the vertebra.

One or more of the screws positioned in the vertebra, or adjacent vertebra(s), may have an expandable distal end. External fixation may include expanding the distal end to form a mesh. External fixation may include placing bone graft on one or more of the plate, screw(s) and rod(s). External fixation may include not placing bone graft on the plate, screw(s) or rod(s).

When the spinal body is a vertebra, external fixation may include driving one or more screws into the vertebra. External fixation may include driving one or more screws into or one or more adjacent vertebras. External fixation may include placing a rod on two adjacent screws. External fixation may include placing a plate on the vertebra. The plate may be fixed to the vertebra by driving or more screws through the plate and into the vertebra. The plate may be plated on a posterior face of the vertebra. The plate may be plated on an anterior side of the vertebra. The plate may be placed on a lateral face of the vertebra. The plate may be placed on a posterolateral face of the vertebra.

One or more of the screws positioned in the vertebra, or adjacent vertebra(s), may have an expandable distal end. External fixation may include expanding the distal end to form a mesh. External fixation may include placing bone graft on one or more of the plate, screw(s) and rod(s). External fixation may include not placing bone graft on the plate, screw(s) or rod(s).

When the spinal body is an intervertebral disc, external fixation may include advancing a screw through the first vertebra, through the second cage, and into the second vertebra.

Prior to or concurrently with the external fixation, the second cage may be filled with cement (step not shown). Filling the second cage may be performed before a delivery handle is removed from the second cage. Filling the second cage may be performed after removal of the delivery handle and after coupling a different handle to the second cage.

At step 122 the method may include surgically closing the access hole.

A method in accordance with the invention may include all of the steps illustrated in method 100. A method in accordance with the invention may include some of the steps illustrated in method 100.

A method in accordance with the invention may some or all of the steps illustrated in method 100 and one or more steps not illustrated in method 100 but described herein.

A method in accordance with the invention may include performing the steps illustrated in method 100 in the illustrated order. A method in accordance with the invention may include performing the steps illustrated in method 100 in an order different from the illustrated order.

A method in accordance with the invention may include steps 102, 104, 106, 114, 108, 110, 120 and 122.

A method in accordance with the invention may include steps 102, 104, 106, 108, 110, 120 and 122.

A method in accordance with the invention may include the steps illustrated in method 100 and performed in the following order: steps 102, 104, 106, 114, 108, 110, 112, 116, 118, 120 and 122.

A method in accordance with the invention may include steps 102, 104, 106, 114, 108, 110, 112, 114 (again), 116, 118, 120 and 122. The cavity formed before the augmenting may have a first volume. The cavity formed after the augmenting may have a second volume. The second volume may be greater than the first volume. The augmenting may include expanding the first cage to a third volume. The second volume may be greater than the third volume. The second volume may be equal to the third volume.

A method for treating a spinal body may include steps 102, 104 and 106. The method may include a step of augmenting a height of the spinal body using one or both of patient positioning and a surgical table (not shown). The method may include steps 114, 116, 118, 120 and 122. The method may include additional method steps disclosed herein.

A method for treating a spinal body may include steps 102, 104, 106, 108, 110 and 112. After the first cage is removed in step 112, the method may include the step of filling an interior of the first cage with material (not shown). The material may include bone graft. The material may include bone cement. The interior may be filled with material using any suitable technique known to those skilled in the art. The method may include steps 120 and 122. The method may include additional method steps disclosed herein.

A method for treating a spinal body may include steps 102, 104, 106, 114, 116, 118, 120 and 122. The method may include additional method steps disclosed herein.

Another method for treating a spinal body may include steps 102, 104, 106, 114, 116, 118. Step 118 may include augmenting the height of the spinal body by expanding the second cage. The method may include steps 120 and 122. The method may include additional method steps disclosed herein.

Another method for treating a spinal body may include steps 102, 104, 106, 114, 116, 118. The method may include the step of filling an interior of the second cage with material (not shown). The material may be bone cement. The material may be bone graft. Filling the interior may include not using a balloon. Filling the interior may include using a balloon. The interior may be filled using any suitable technique known to those skilled in the art. The method may include steps 120 and 122. The method may include additional method steps disclosed herein.

A method for treating a spinal body may include steps 102, 104, 106, 114, 116, 118. The method may include the step of not filling an interior of the second cage with material (not shown). The material may be bone cement. The material may be bone graft. The method may include steps 120 and 122. The method may include additional method steps disclosed herein.

A method for treating a spinal body may include steps 102, 104, 106 and 114. The method may include filling the cavity with material. The material may include bone graft. The material may include bone cement. The interior may be filled with material using any suitable technique known to those skilled in the art. The method may also include steps 120 and 122. The method may include additional method steps disclosed herein.

A method for treating a spinal body may include augmenting a height of the spinal body using patient positioning or a surgical table. The method may include step 120 prior to positioning an implant in the interior. When the spinal body is a vertebra, pedicle screws, rods and/or plates may be inserted into the vertebra before an implant is positioned in the disc space. When the spinal body is an interbody disc, pedicle screws, rods and/or plates may be inserted into the adjacent first and second vertebras before an implant is positioned in the disc space. The method may include steps 102, 104, 106, 114, 116, 118 and 122. The second implant, when expanded in the disc space, may provide support, from within the disc space, to maintain the augmented height of the spinal body.

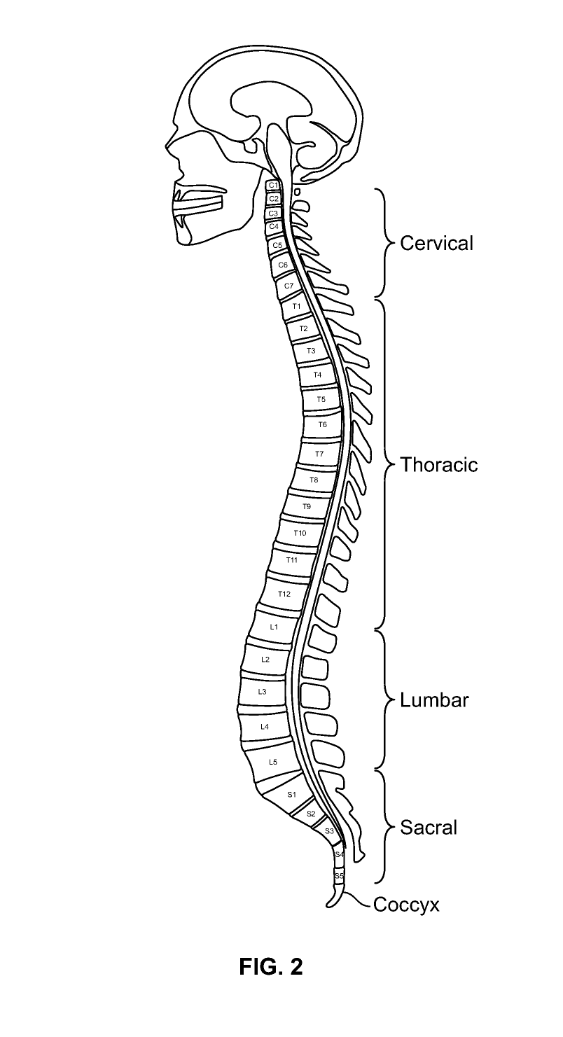

FIG. 2 shows illustrative anatomy in connection with which the apparatus and methods may be used. FIG. 2 shows an illustrative portion of a human skeleton including the spine. The spine may include illustrative cervical vertebra C1-C7, thoracic vertebra T1-T12, lumber vertebra L1-L5, sacral vertebra S1-S5 and the coccyx.

The methods may be performed in a cervical vertebra. The methods may be performed in a thoracic vertebra. The methods may be performed in a lumbar vertebra. The methods may be performed in a sacral vertebra. The methods may be performed in the coccyx. Methods performed herein may be performed in two or more vertebrae illustrated in FIG. 2.

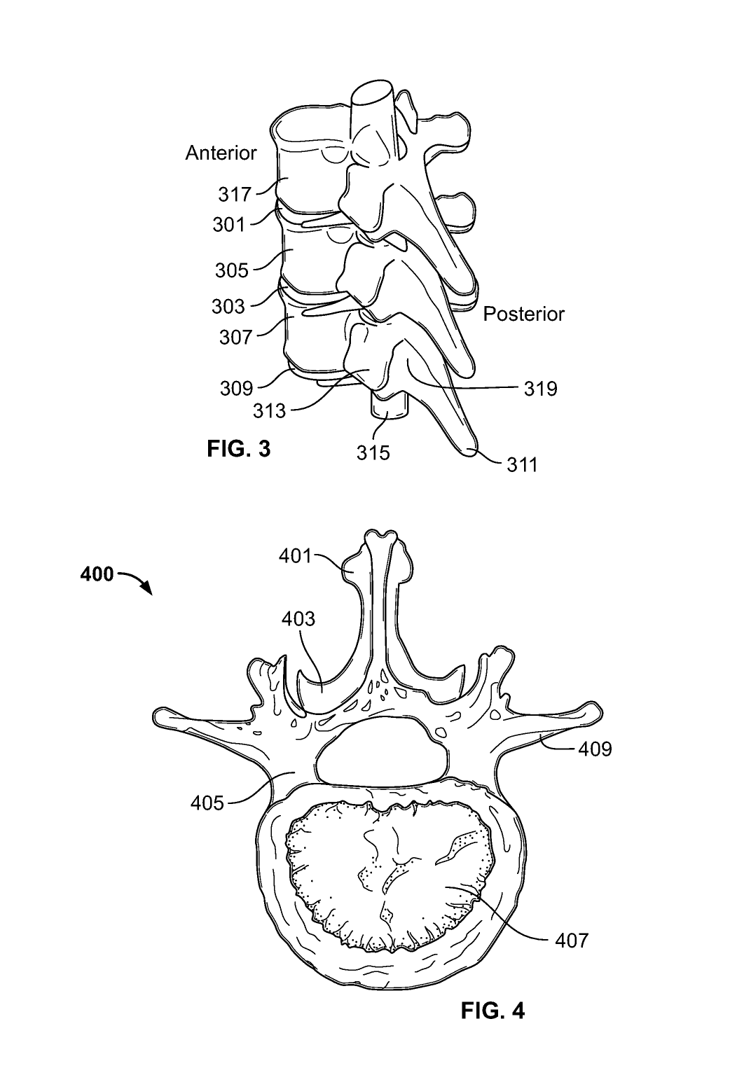

FIG. 3 shows an illustrative view of a portion of the spine. The illustrative view includes vertebras 317, 305 and 307. The illustrative view includes intervertebral discs 301, 303 and 309. Disc 301 is disposed between vertebra 317 and vertebra 305. Vertebra 303 is disposed between vertebra 305 and 307. Intervertebral disc 301 is disposed between vertebra 317 and vertebra 305. Intervertebral disc 301 is disposed adjacent vertebra 317. Intervertebral disc 301 is disposed adjacent vertebra 305.

The illustrative view includes spinal code 315 passing along a posterior face of vertebras 317, 305 and 307 and intervertebral discs 301, 303 and 309.

Vertebra 307 includes transverse process 313, spinous process 311 and lamina 319.

FIG. 4 shows a top plan view of illustrative vertebra 400. Vertebra 400 includes spinous process 401, transverse process 409, lamina 403, pedicle 405 and vertebral body 407.

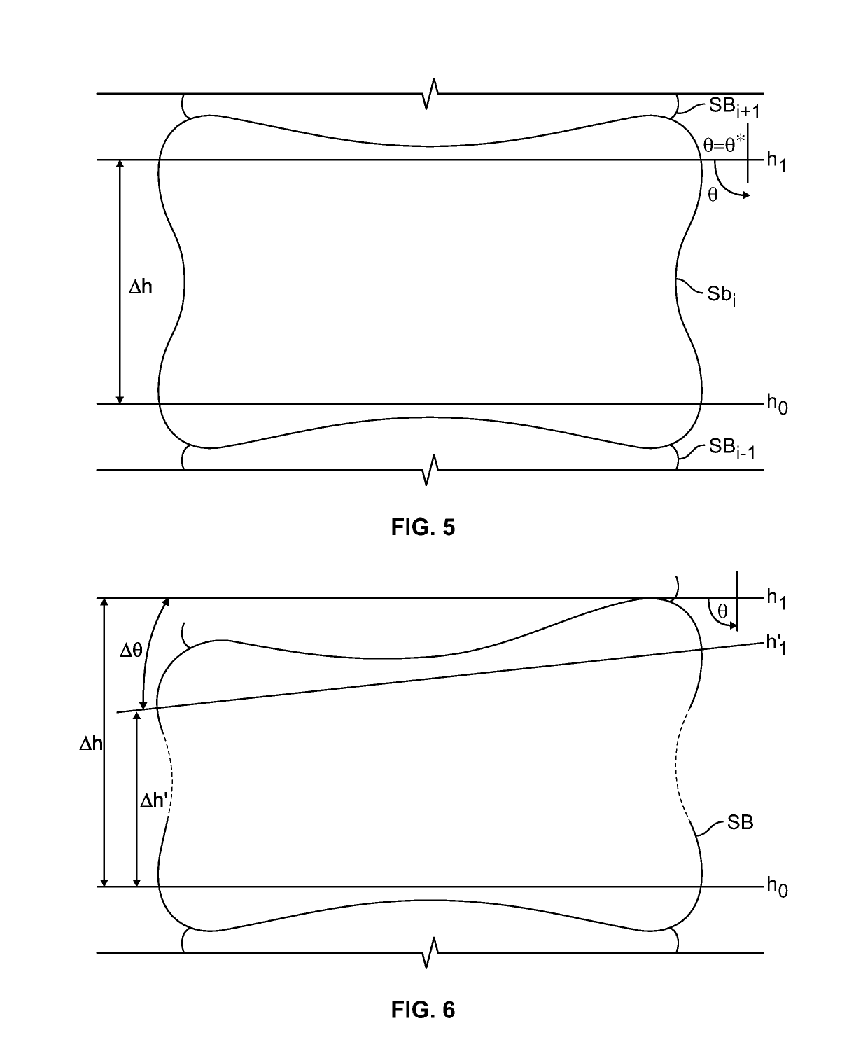

FIGS. 5-8 shows spinal body SBi vertically positioned above spinal body SBi−1 and below spinal body SBi+1. SBi is illustrated as a vertebra, and SBi−1 and SBi+1 as intervertebral discs, but the principles shown may be applied to anatomy in which SBi is an intervertebral disc and SBi−1 and SBi+1 are vertebra.

FIG. 5 shows reference horizons h0 and h1 for the bottom and top of SBi, respectively. h0 and h1 are oriented parallel to each other at q=q*, which is defined as a “normal” orientation of h0 and h1. h0 and h1 may have “normal” orientations q* that are different from each other. h0 and h1 may have “normal” orientations q* that are different from zero SBi has height Dh.

FIG. 6 shows a degenerate condition in SBi. h1 has moved down and has rotated through Dq. SBi now has height Dh′, which may vary from one side of SBi to the other. Some degenerate conditions may have a uniform reduction of height from one side of SBi to the other. Some degenerate conditions may have only a change in the angle of h1.

FIG. 7 shows illustrative cage expansion A for restoring SBi to h1. A is a broken line that crosses the boundary of SBi. An actual cage expansion may deform under the load applied to SBi, as shown in illustrative cage expansion A′.

The illustrative cage may be a single-layered cage. The cage may be the first cage. The illustrative cage may be the second cage.

FIG. 8 shows expansions B and C of two cages, one inside the other. The two cages, one inside the other, may form a double-layered cage. The cage may be the first cage. The cage may be the second cage.

Expansion B may be an expansion like expansion A′ (shown in FIG. 7). Expansion B may be an expansion that undergoes less deformation than that shown in FIG. 7. Expansion C of the inner cage may buttress expansion B.

The shape of a cage, such as the shape of the first cage, the second cage, the cage illustrated in FIG. 7, or the cage illustrated in FIG. 8, may impact on the radial or hoop strength of the cage. Cell patterns, wall thickness, and strut dimensions of a cage may be varied to enhance the radial or hoop strength of the cage. Additionally, the cage may be strengthened by adding additional layers to the construct. For example, the cage may be double-layered. The cage may be triple layered. The inner layer(s) may exhibit a different shape than an outer layer of the cage. The inner layer(s) may have a larger radial or hoop force than the outer layer, adding radial or hoop strength to the cage.

The outer layer may have an outer layer profile. The outer layer profile may be convex. The outer layer may be concave. The profile may match the anatomy of an interior of a spinal body in which the cage may be implanted. The profile may match the size and shape of a cavity prepared in the interior of the spinal body.

The inner layer may be round and symmetric. The inner layer may have an asymmetric shape.

A layer of a cage may have a constant cell density. A layer of a cage may have a varying cell density. A layer of a cage may define two or more cell densities.

Methods in accordance with the invention are described below. The shape of apparatus shown in the figures may be illustrative. An illustrated shape of apparatus may not account for the deformation, deflection, compression, torsion, and/or any other force that may be applied to the apparatus during the method.

A shape of an expanded cage may be shown schematically. A shape of an expanded broaching member may be shown schematically.

The methods illustrated below are shown as being performed with single-layer cages. Alternatively, one or more of the cages may be double-layered. One or more of the cages may be triple-layered. A double-layer cage may include two laser-cut tubes. A triple-layer cage may include three laser-cut tubes. FIG. 7 (above) shows exemplary expansion and deformation of a single-layered cage under a load applied to the single-layered cage. FIG. 8 (above) shows exemplary expansion and deformation of a double-layered cage under a load applied to the double-layered cage.

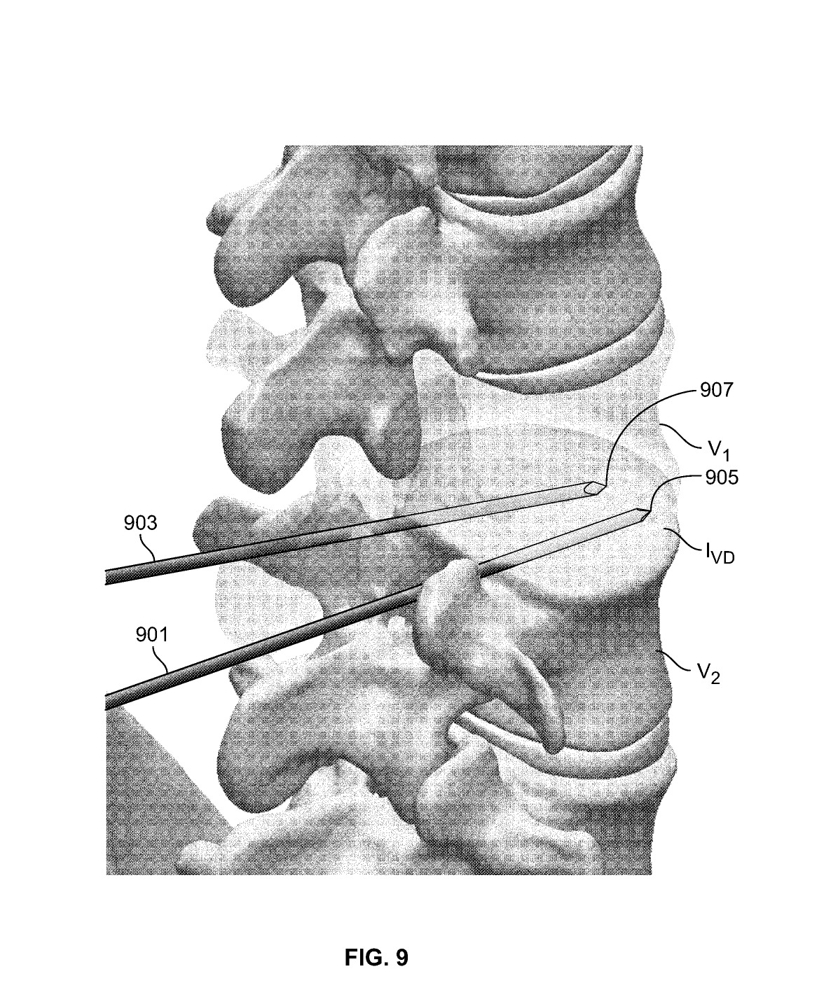

FIGS. 9-17 show an illustrative method for treating a spinal body. The spinal body may be intervertebral disc Ivd (“Ivd”). Ivd may be disposed between vertebra V1 and vertebra V2. The method illustrated in FIGS. 9-17 may include additional method steps disclosed herein. The method may be performed in the order shown. The method may be performed in an order different from the order shown. The method may involve one or more of some or all of the apparatus shown in FIGS. 9-17.

The method illustrated in FIGS. 9-17 is performed using a bilateral approach. The method shown in FIGS. 9-17 may be performed using a unilateral approach.

In FIGS. 9-17, Ivd is accessed through a posterior side of Ivd. Accessing Ivd through the posterior side may be performed during a Posterior Lumbar Interbody Fusion (“PLIF”) procedure. Accessing Ivd through the posterior side may be performed during an Oblique Lumbar Interbody Fusion (“OLIF”) procedure.

The method illustrated in FIGS. 9-17 may be performed using an extrapedicular approach. The method illustrated in FIGS. 9-17 may be performed using a transpedicular approach. The method illustrated in FIGS. 9-17 may be performed using both an extrapedicular and a transpedicular approach.

FIG. 9 shows illustrative posterior placement of guide wire 901 and guide wire 903 in Ivd. A tip of guide wire 901 may be positioned at target site 905. A tip of guide wire 903 may be positioned at target site 907. Guide wires 901 and 903 may be inserted into Ivd at a desired trajectory and depth. Fluoroscopy may be used in an anterior-posterior plate. Fluoroscopy may be used in one or both lateral planes.

Guide wires 901 and 903 may be inserted into Ivd during a bilateral procedure. Guide wire 901 may be inserted during a unilateral procedure. Guide wire 903 may be inserted into Ivd during a unilateral procedure.

Guide wires 901 and 903 may be inserted into multiple intervertebral discs (not shown). Guide wire 901 may be inserted into Ivd at a first elevation. Guide wire 903 may be inserted into Ivd at a second elevation. The first elevation may be the same as the second elevation. The first elevation may be different from the second elevation.

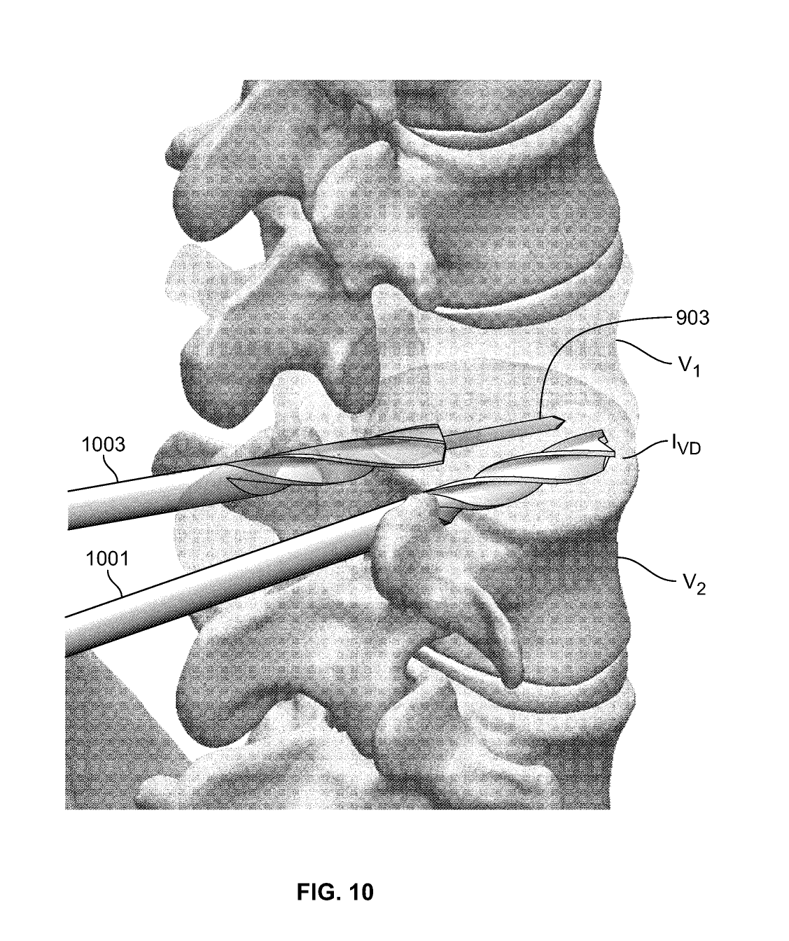

FIG. 10 shows illustrative drilling along guide wires 901 and 903. Drill 1001 may be advanced along guide wire 901 to target site 905. Drill 1003 may be advanced along guide wire 903 to target site 907. A trajectory of the drilling may be set by the position guide wires 901 and 903. The drilling may continue to a depth in Ivd. The depth may be determined by using fluoroscopy or instrumentation.

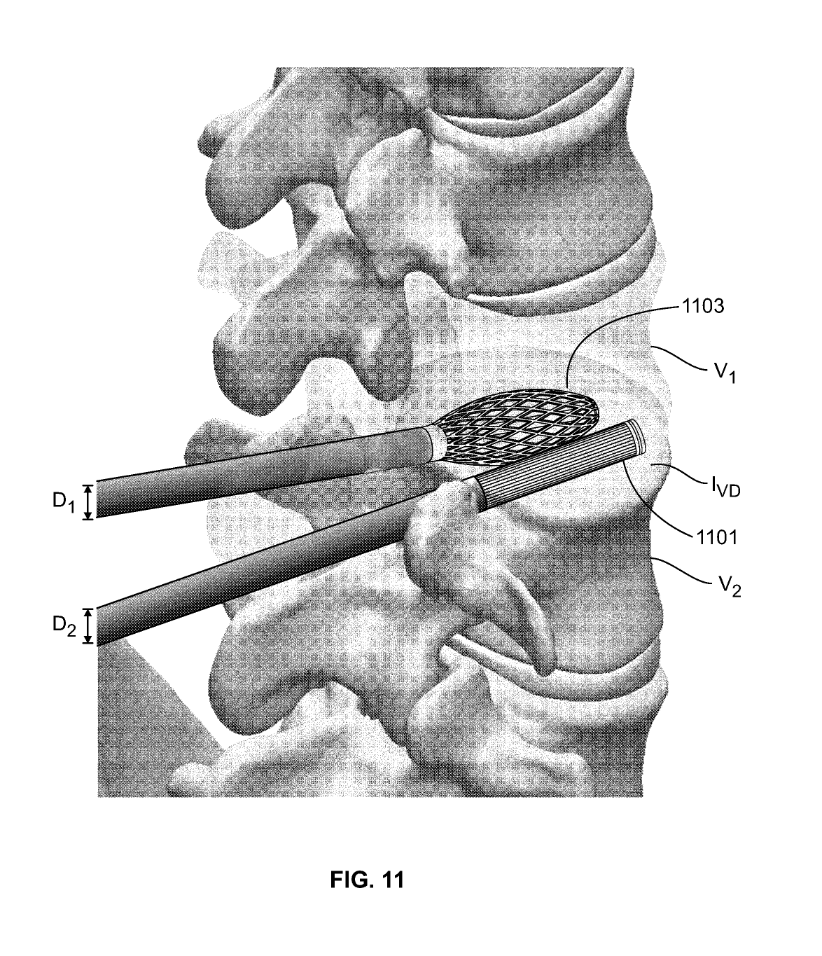

FIG. 11 shows illustrative positioning of unexpanded cage 1101 in Ivd. FIG. 11 illustrates cage 1103, expanded, and positioned in Ivd. The pattern used to represent the unexpanded cage may be schematic.

Cage 1101 may have one or more features in common with the first cage. Cage 1101 may have one or more features in common with the second cage. Cage 1101 may have one or more features in common with the first cage. Cage 1103 may have one or more features in common with the second cage.

FIG. 11 shows illustrative augmenting of a height of Ivd (change in disc height not illustrated). The height of Ivd may be augmented by expanding cage 1103 in Ivd. Expanding cage 1103 may alleviate nerve compression or other pathology. Cage 1103 may be expanded to a first volume.

Cage 1101 may be expanded to a second volume (not shown). The first volume may be equal to the second volume. The first volume may be different from the second volume. Expanding cage 1101 may augment a height of Ivd.

Cage 1101 may be advanced into Ivd through a diameter of a drill hole prepared by drill 1001. Cage 1103 may be advanced into Ivd through a diameter of a drill hole prepared by drill 1003.

Cages 1101 and 1103 may be expandable mesh cages. One or both of cages 1101 and 1103 may be an implant. One or both of cages 1101 and 1103 may be formed from non-implantable material. One or both of cages 1101 and 1103 may be a distraction tool. One or both of cages 1101 and 1103 may be a cavity preparation instrument (not shown).

After augmentation of the height of Ivd, one or more pedicle screws may be advanced into one or both of V1 and V2. The pedicle screws may be provisionally tightened to hold the augmented height in place.

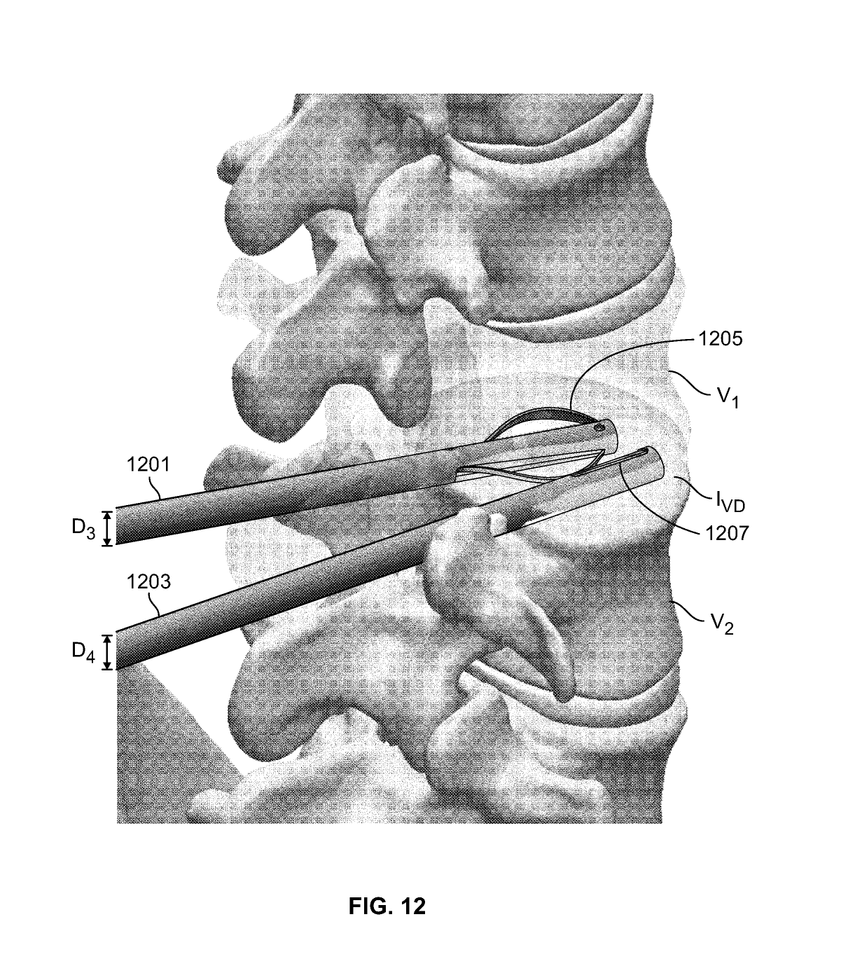

Cages 1101 and 1103 may be removed from Ivd after augmenting the height of Ivd to a desired distraction. The method may proceed with the step illustrated in FIG. 12. Cages 1101 and 1103 may be implanted in Ivd. External fixation mechanisms described herein may be used to maintain the augmented height of Ivd.

Cages 1101 and 1103 may be used to prepare a cavity preparation in Ivd. Cages 1101 and 1103 may be circumferentially rotated to prepare the cavity. Cages 1101 and 1103 may include one or more cutting edges.

FIG. 12 shows illustrative cavity preparation using cavity preparation instrument 1201 and cavity preparation instrument 1203.

In FIG. 12, cavity preparation instrument 1203 is shown with broaching member 1207 in a collapsed state. Cavity preparation instrument 1201 is shown with broaching member 1205 activated. Fluoroscopy and tactile feedback may be used by a practitioner to judge depth and size of disc dissection.

Cavity preparation instrument 1201 may be the cavity preparation instrument. Broaching member 1205 may be the broaching member.

Cavity preparation instrument 1201 may form a first cavity within Ivd by being rotated about a central axis of cavity preparation instrument 1201. Rotating cavity preparation instrument 1201 may displace bone material in Ivd. Rotating cavity preparation instrument 1201 may distract and remove disc material in Ivd to form a cavity for an implant. Rotating cavity preparation instrument 1201 may also prepare a vertebral end plate of V1 and a vertebral end plate of V2 for implantation by scraping the end plates with the broaching member, thereby promoting bleeding bone. Bleeding bone may be desirable for its aid in promoting fusion.

A practitioner may activate broaching member 1207 to prepare a second cavity within Ivd (not shown)

After the first and second cavities are prepared, broaching members 1205 and 1207 may be de-activated and removed from Ivd.

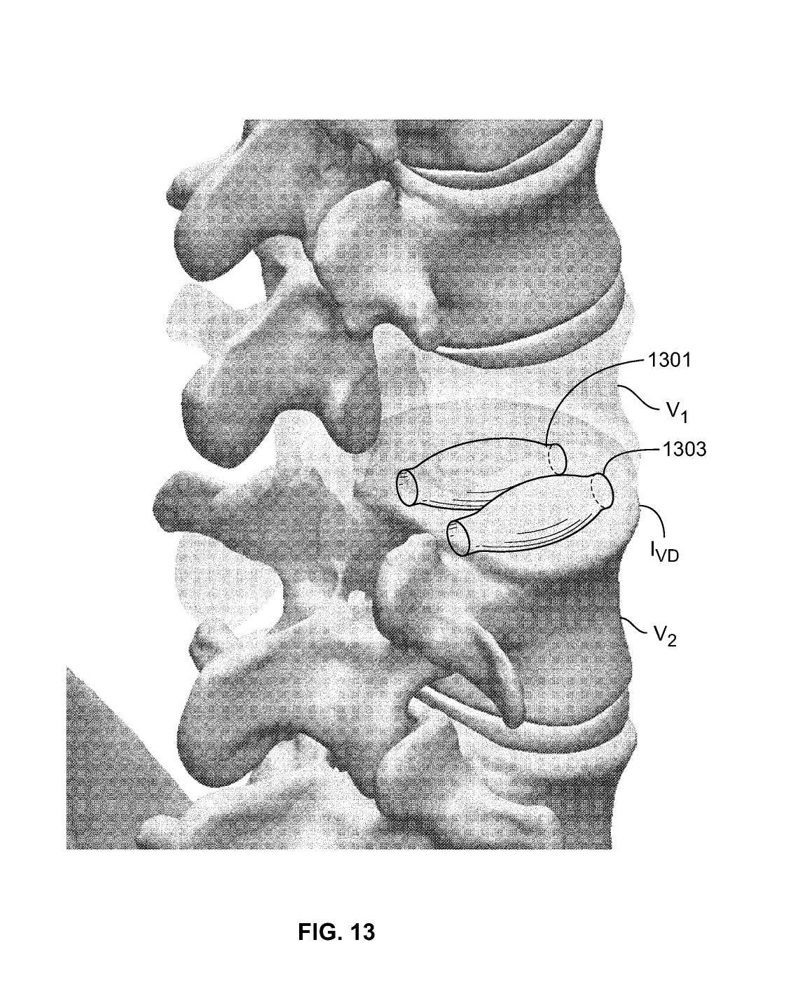

FIG. 13 shows illustrative cavity 1301 in Ivd. Cavity 1301 may be prepared using cavity preparation instrument 1201. FIG. 13 shows illustrative cavity 1303 in Ivd. Cavity 1303 may be prepared by cavity preparation instrument 1203.

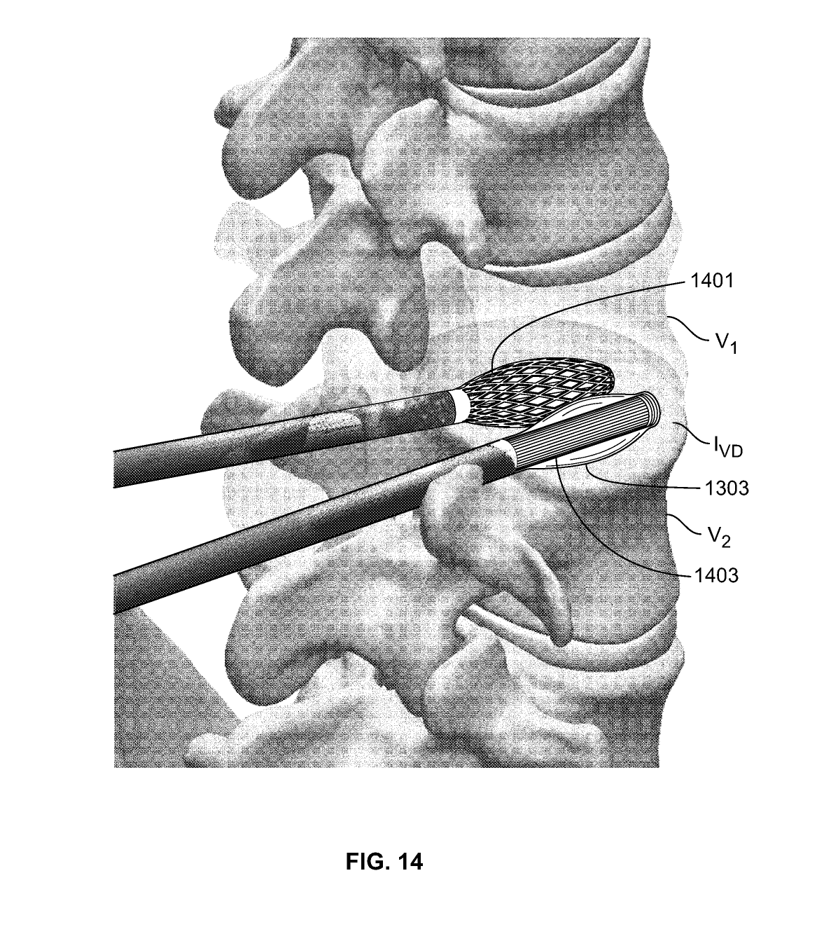

FIG. 14 shows illustrative positioning of unexpanded cage 1403 in Ivd and within cavity 1303. FIG. 14 also shows illustrative positioning of cage 1401 in cavity 1301. Cage 1401 is shown in its expanded state.

Cage 1401 may have one or more features in common with the first cage. Cage 1401 may have one or more features in common with the second cage. Cage 1403 may have one or more features in common with the first cage. Cage 1403 may have one or more features in common with the second cage.

The method illustrated in FIG. 14 may include inserting cage 1401, in a collapsed state, into cavity 1301. Cage 1401 may be advanced through a first cage delivery system and into cavity 1301. The method may include inserting cage 1403, in the collapsed state, into cavity 1303. Cage 1403 may be advanced through a second cage delivery instrument system and into cavity 1303. The method may include deploying cage 1401 and 1403. Deploying a cage may include expanding the cage.

Expanding cage 1401 may include rotating cage 1401 by engaging a tail of the cage. Expanding cage 1401 within the cavity may pack an interior of cage with disc material and further prepare vertebral end plates to the contour of cage 1401. Cage 1401 may be packed with one or more of bone, biologics, and/or cement before deployment.

Expanding cage 1403 may include rotating cage 1403 by engaging a tail of the cage. Expanding cage 1403 within the cavity may pack an interior of cage with disc material and further prepare vertebral end plates to the contour of cage 1403. Cage 1403 may be packed with one or more of bone, biologics, and/or cement before deployment.

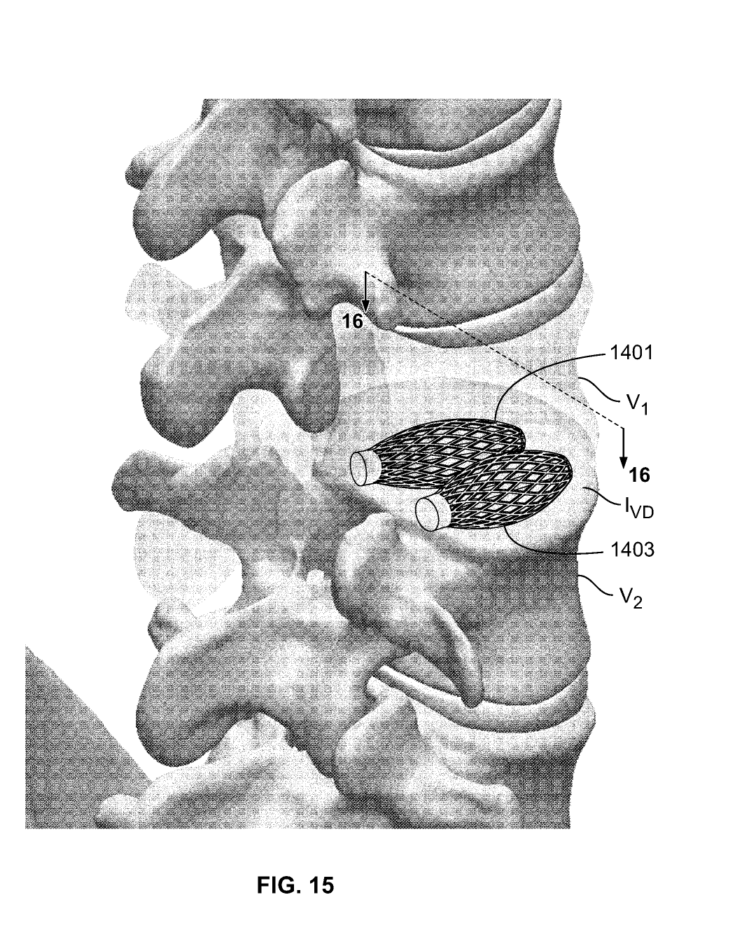

FIG. 15 shows illustrative bilateral placement of cages 1401 and 1403 in Ivd. Cages 1401 and 1403 may be positioned at a single level or position on the spine. FIG. 15 shows cages 1401 and 1403 after a handle has been removed from each of cages 1401 and 1403.

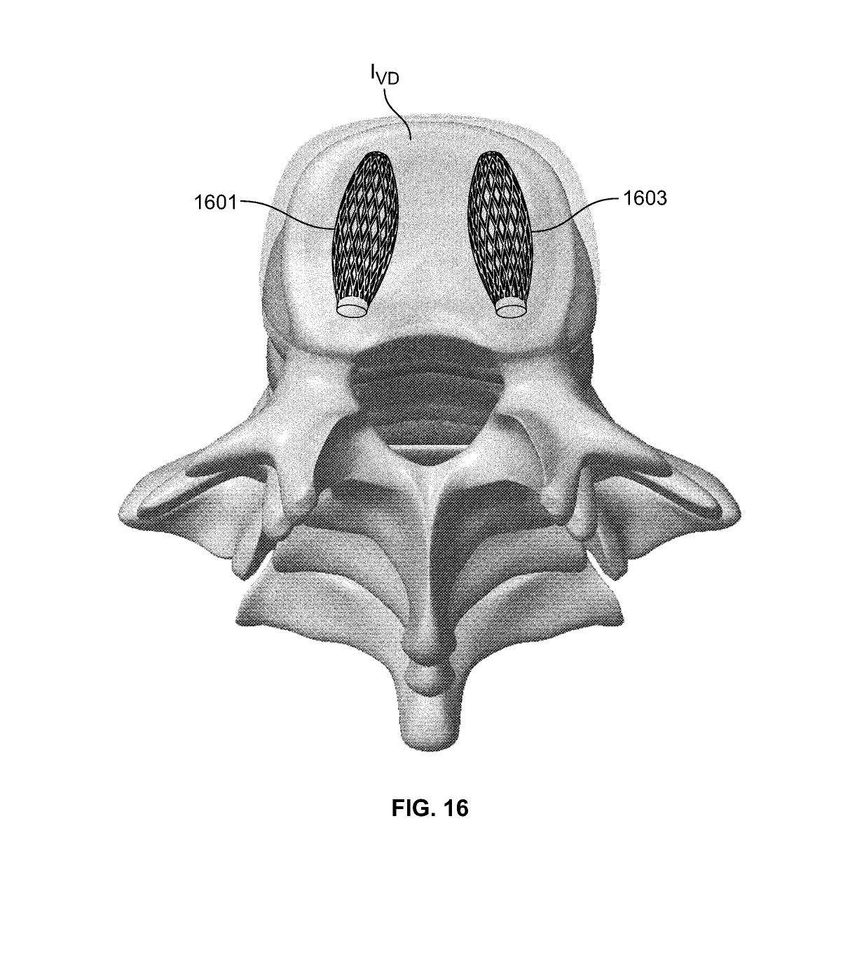

FIG. 16 shows an illustrative view of Ivd taken along cut lines 16-16, with V1 not being shown. FIG. 16 shows an illustrative view of cages 1601 and 1603 deployed in Ivd. Cages 1601 and 1603 may be cages 1401 and 1403. Cages 1601 and 1603 may be cages different from cages 1401 and 1403.

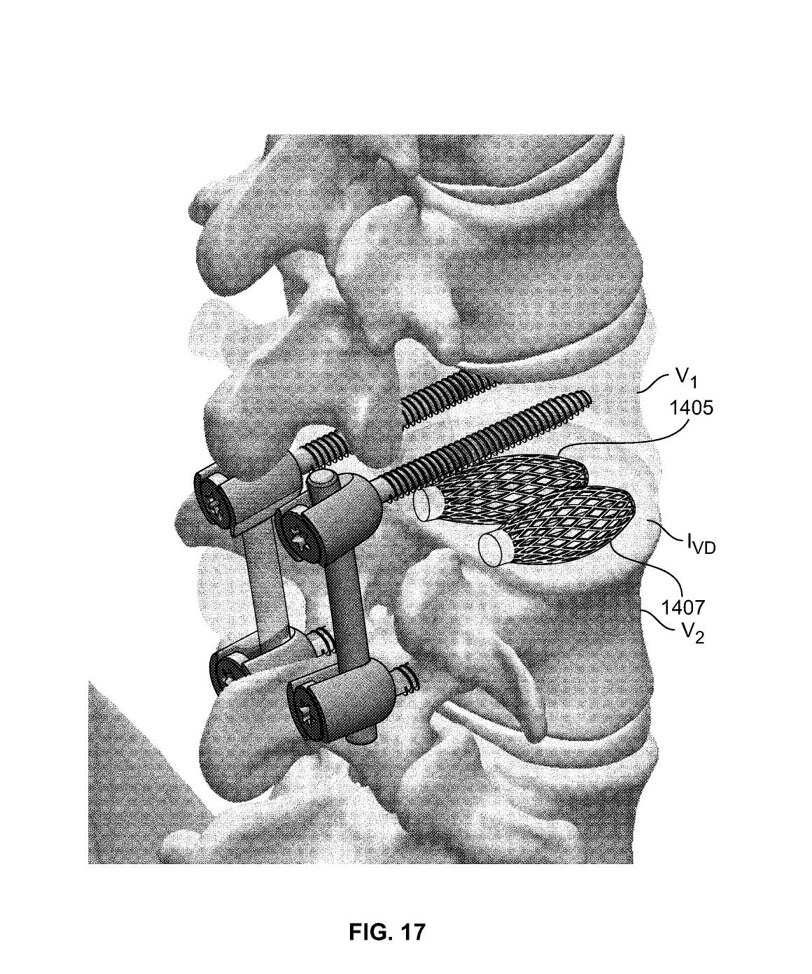

FIG. 17 shows illustrative supplemental fixation including bilateral placement of pedicle screws on V1 and V2. The supplemental fixation may include driving pedicle screws into V1. The supplemental fixation may include driving pedicle screws into V2. Unilateral placement of pedicle screws on V1 and V2 may be performed. The pedicle screws may be locked with rods.

Additional compression may be applied to the implants by compressing the vertebral bodies and locking pedicle screws to rods. Additional compression may be used to tightly position the cage between V1 and V2, reducing the possibility of the cage migrating away from an implanted position.

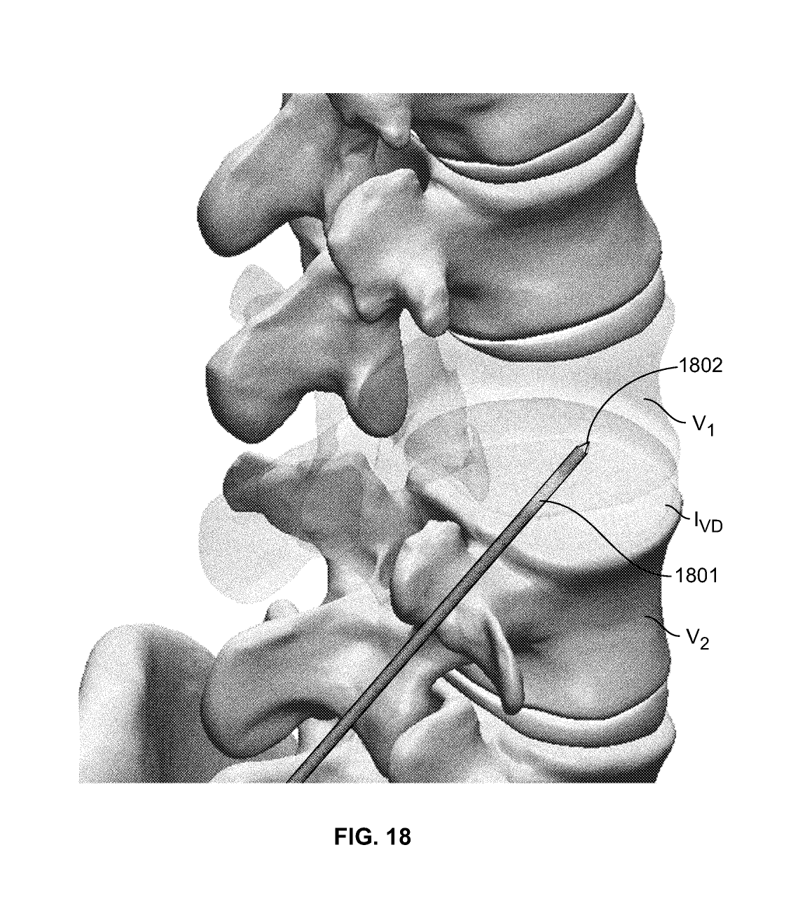

FIGS. 18-29 show an illustrative method for treating a spinal body. The spinal body may be intervertebral disc Ivd (“Ivd”). Ivd may be disposed between vertebra V1 and vertebra V2. The method illustrated in FIGS. 18-29 may include additional method steps disclosed herein. The method may be performed in the order shown. The method may be performed in an order different from the order shown. The method may involve one or more of some or all of the apparatus shown in FIGS. 18-29.

The method illustrated in FIGS. 18-29 is shown using a unilateral approach. The method shown in 18-29 may be performed using a bilateral approach.

In FIGS. 18-29, Ivd is accessed through a posterior side of Ivd. Ivd may be accessed through a posterior side during a Transforaminal Lumbar Interbody Fusion (“TLIF”) procedure.

FIG. 18 shows illustrative posterior placement of guide wire 1801 at target site 1802 in Ivd. Guide wire 1801 may be placed in Ivd at a desired trajectory and depth. Fluoroscopy may be used in both anterior-posterior and lateral planes to confirm placement. Guide wires may be inserted at multiple levels, or elevations, along the spine (not shown).

The method illustrated in FIGS. 18-29 may be performed using an extrapedicular approach. The method illustrated in FIGS. 18-29 may be performed using a transpedicular approach. The method illustrated in FIGS. 18-29 may be performed using both an extrapedicular and a transpedicular approach.

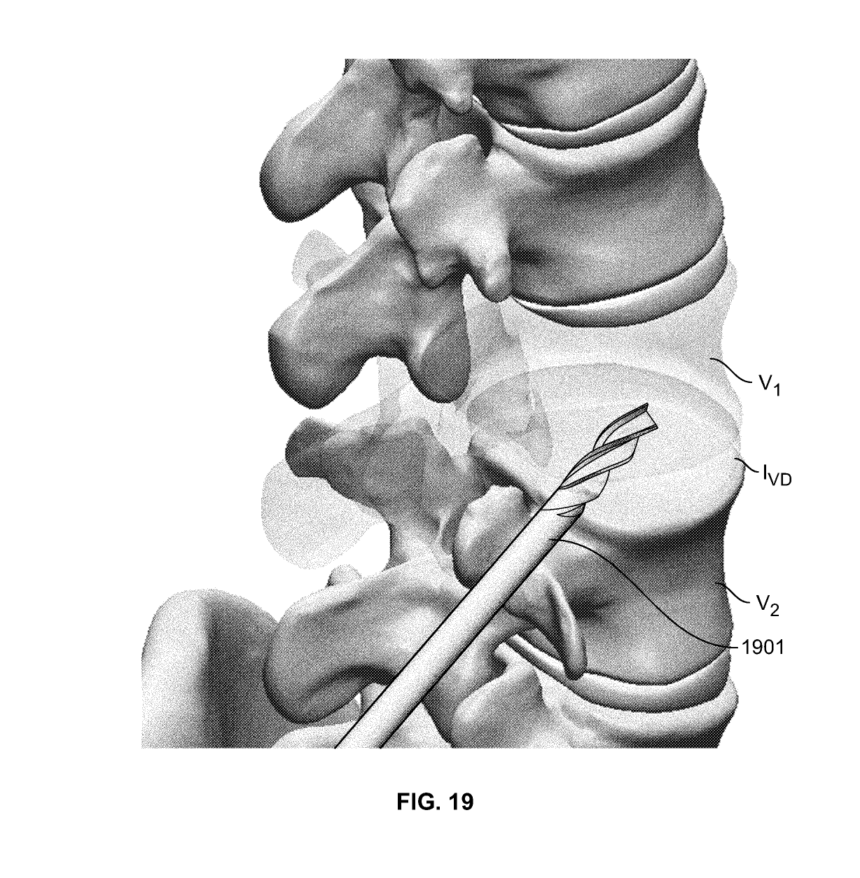

FIG. 19 shows illustrative drilling along an access path defined by guide wire 1801 to target site 1802. Drill 1901 may drill along the access path. Drill 1901 may be cannulated. Drill 1901 may be advanced along guide wire 1801 to target site 1802. Drill 1901 may be solid. Guide wire 1801 may be removed from Ivd prior to advancing drill 1901 along the access path and to target site 1802. The drilling may be performed to a depth in Ivd. The depth may be determined by using fluoroscopy and or instrumentation.

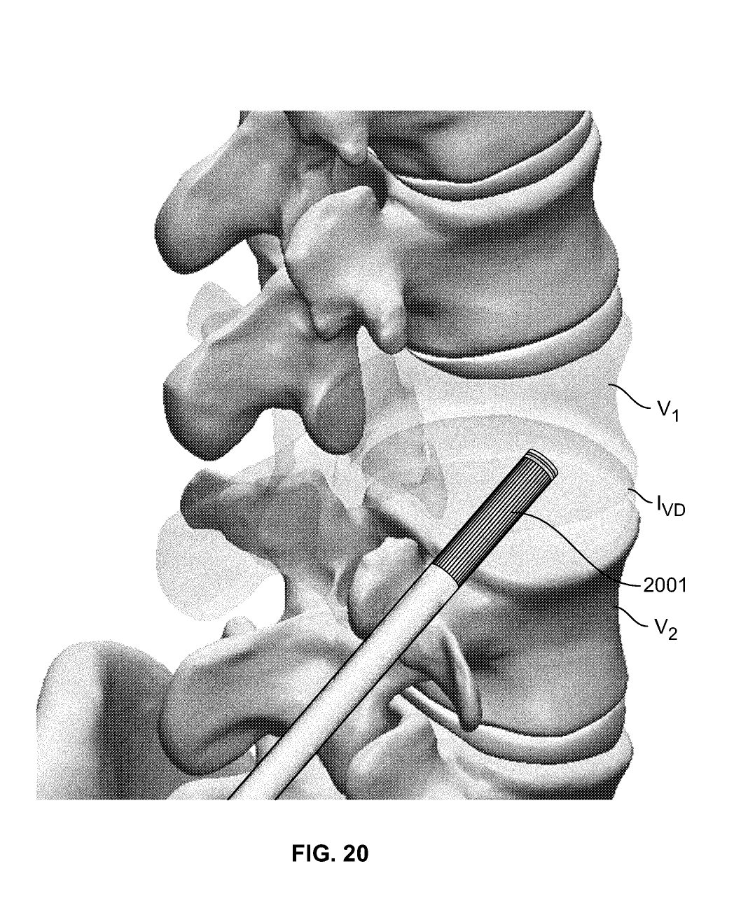

FIG. 20 shows illustrative placement of cage 2001 in Ivd. Cage 2001 is placed in Ivd in an unexpanded state. Cage 2001 may be advanced into Ivd through a diameter of a drill hole prepared by drill 1901.

Cage 2001 may have one or more features in common with the first cage. Cage 2001 may have one or more features in common with the second cage.

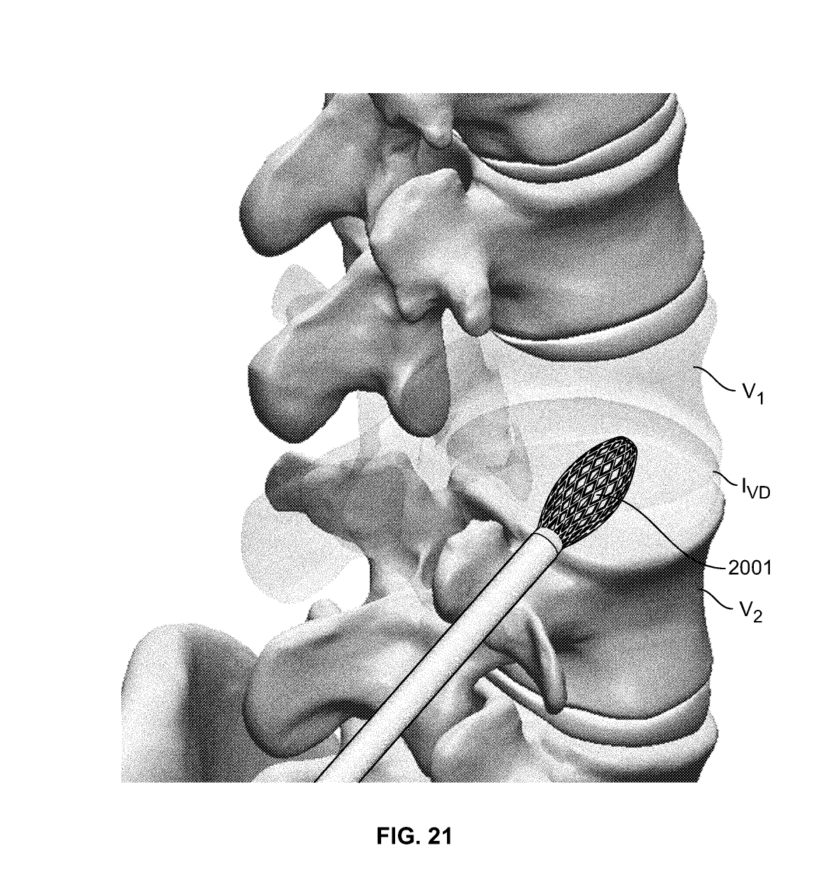

FIG. 21 shows illustrative augmenting of a height of Ivd (change in disc height not illustrated). The height of Ivd may be augmented by expanding cage 2001 in Ivd. Expanding cage 2001 may alleviate nerve compression or other pathology.

Cages 2001 may be an expandable mesh cage. Cage 2001 may be an implant. Cage 2001 may be formed from non-implantable material. Cage 2001 may be a distraction tool. Cage 2001 may be a cavity preparation instrument (not shown).

After augmentation of the height of Ivd, one or more pedicle screws may be advanced into one or both of V1 and V2. The pedicle screws may be provisionally tightened to hold the augmented height in place.

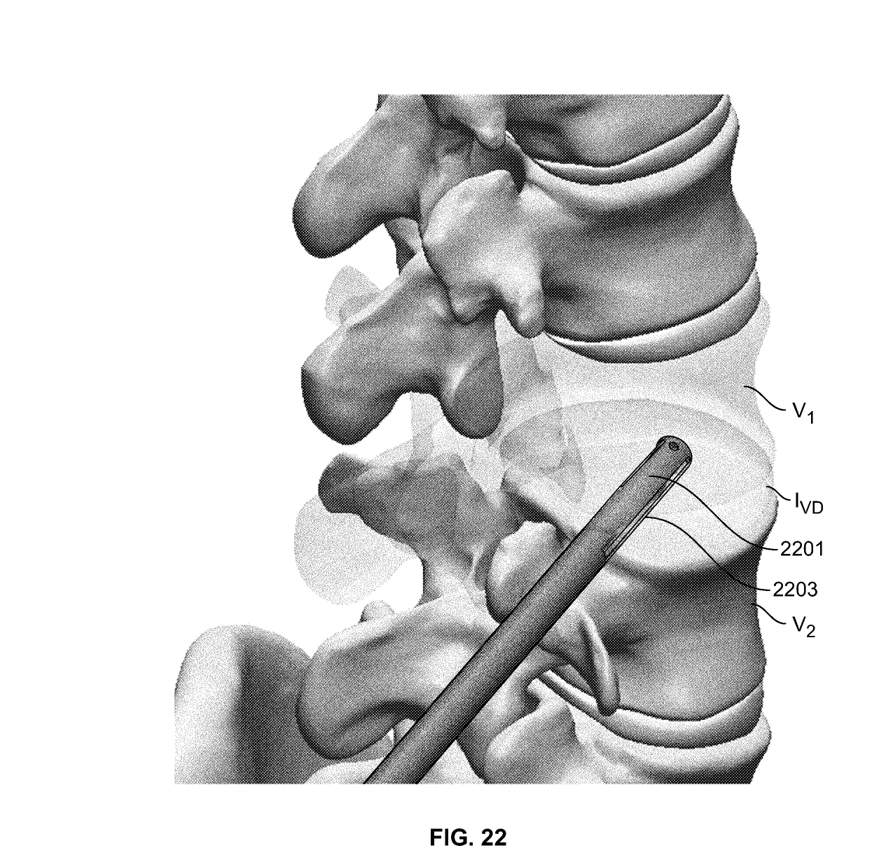

Cages 2001 may be removed from Ivd after augmenting the height of Ivd to a desired distraction. The method may proceed with the step illustrated in FIG. 22. Cage 2001 may be implanted in Ivd. External fixation mechanisms described herein may be used to maintain the augmented height of Ivd.

Cage 2001 may be used to prepare a cavity in Ivd. Cage 2001 may be circumferentially rotated to prepare the cavity. Cage 2001 may include one or more cutting edges.

FIG. 22 shows illustrative positioning of cavity preparation instrument 2203 in Ivd. Broaching member 2203 is shown in a collapsed state. Cavity preparation instrument 22013 may be the cavity preparation instrument. Broaching member 2203 may be the broaching member.

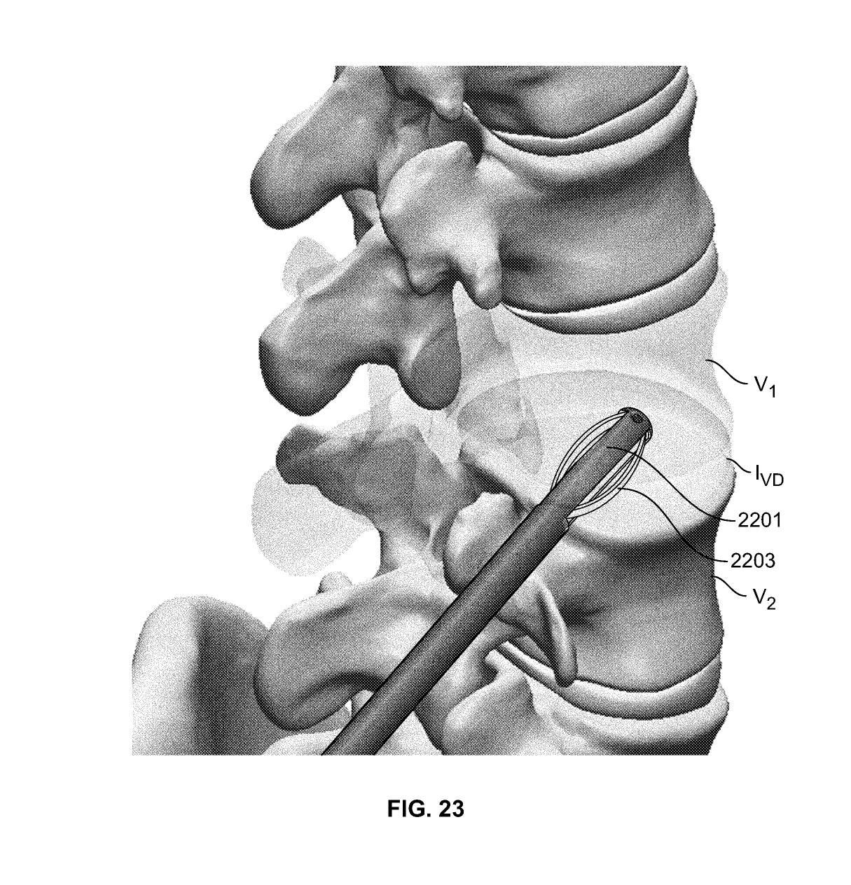

FIG. 23 shows illustrative preparation of a cavity in Ivd. In FIG. 23, broaching member 2203 has been activated. Rotating the broaching member in Ivd may cut bone material. Rotating the broaching member in Ivd may distract and remove disc material in Ivd. Fluoroscopy and tactile feedback may be used by a practitioner to judge a depth and size of disc dissection. Rotating broaching member in Ivd may prepare a vertebral end plate of V1 and a vertebral end plate of V2 for implantation of a cage. Broaching member may prepare the end plate for implantation by scraping the end plates and thereby promoting bleeding bone.

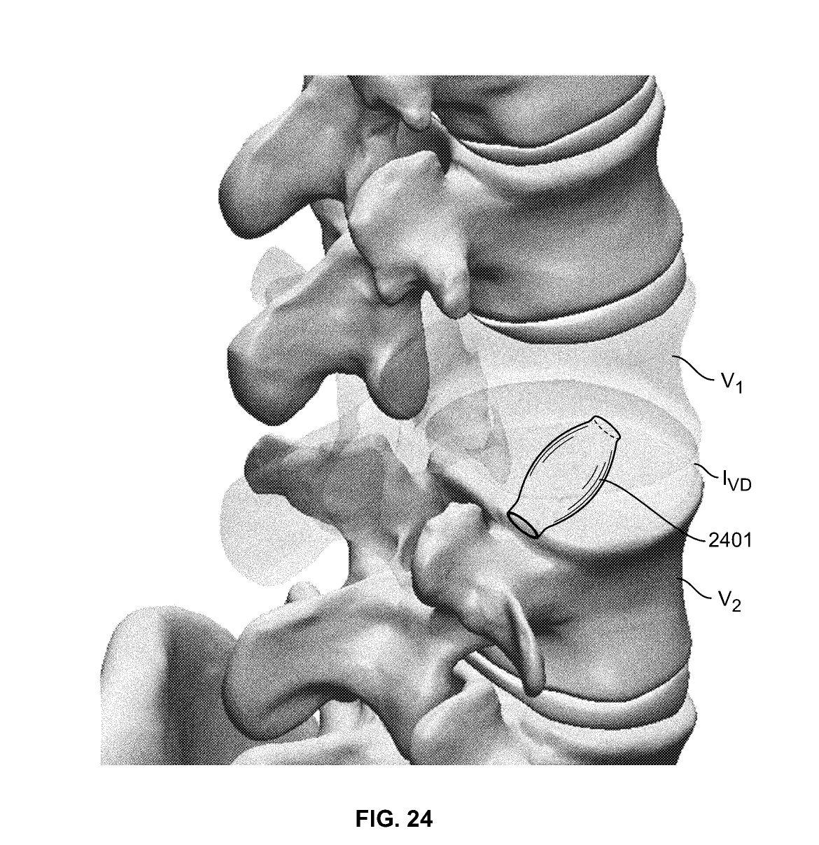

FIG. 24 shows an illustrative view of cavity 2401. Cavity 2401 may be prepared by cavity preparation instrument 2201.

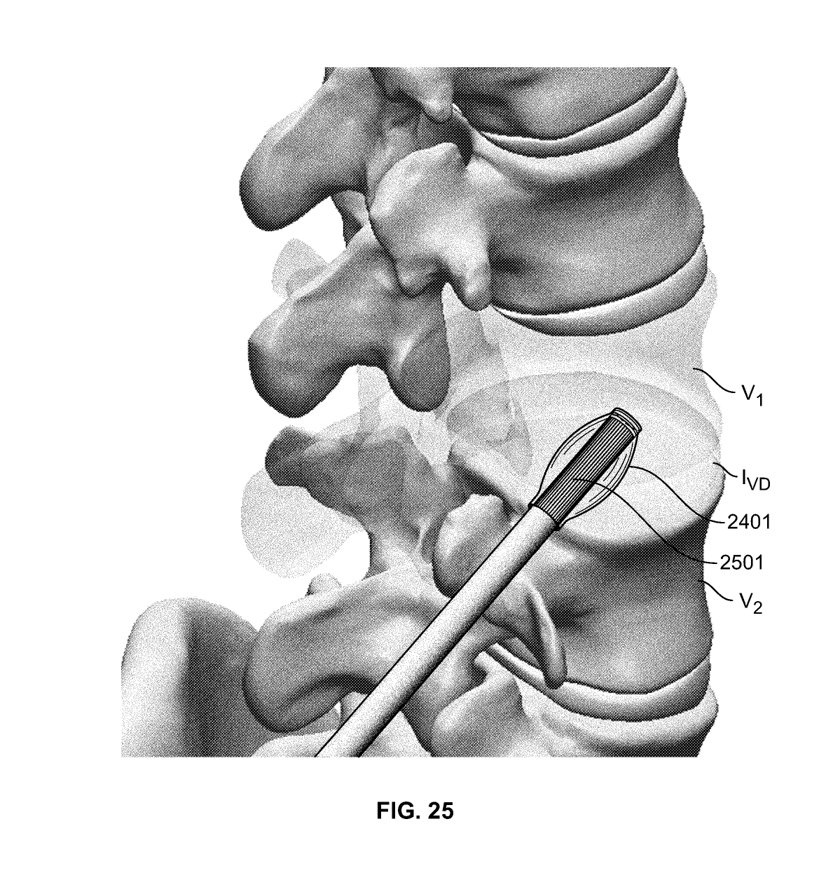

FIG. 25 shows illustrative positioning of cage 2501, in an unexpanded state, in cavity 2401. Cage 2501 may have one or more features in common with the first cage. Cage 2501 may have one or more features in common with the second cage.

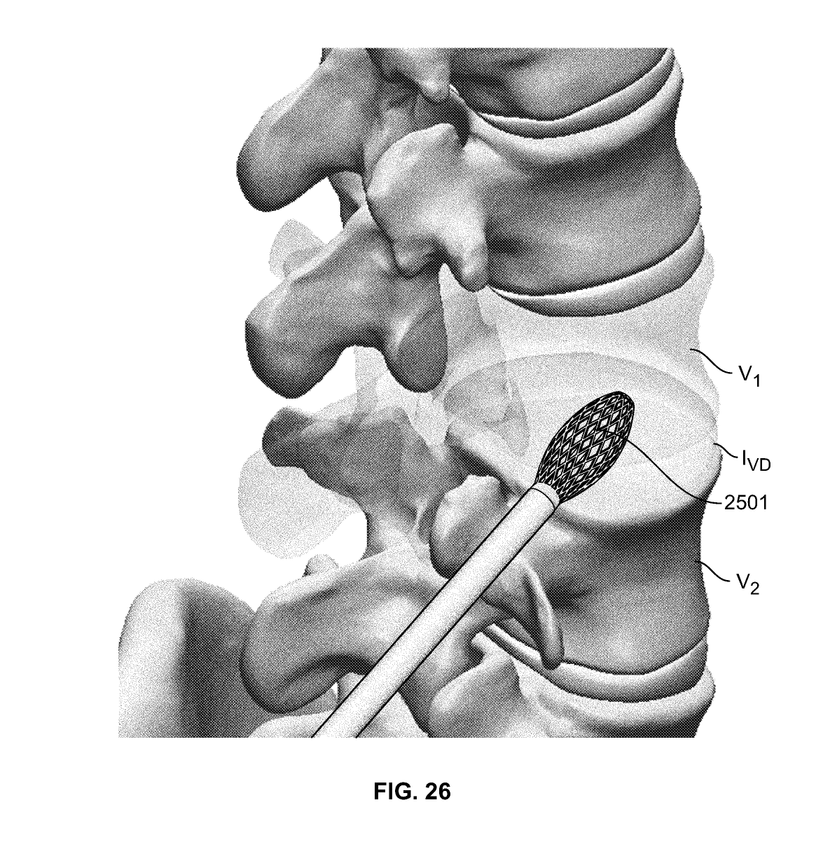

FIG. 26 shows illustrative expansion of cage 2501 in cavity 2401 to fill cavity 2401.

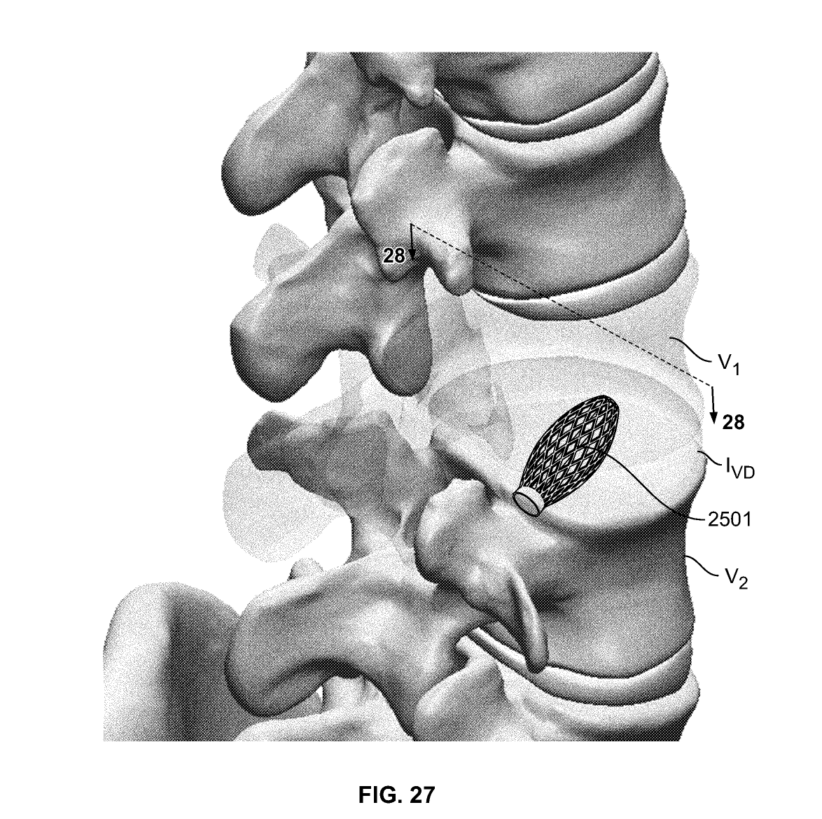

FIG. 27 shows cage 2501 in Ivd after a handle of cage 2501 has been removed.

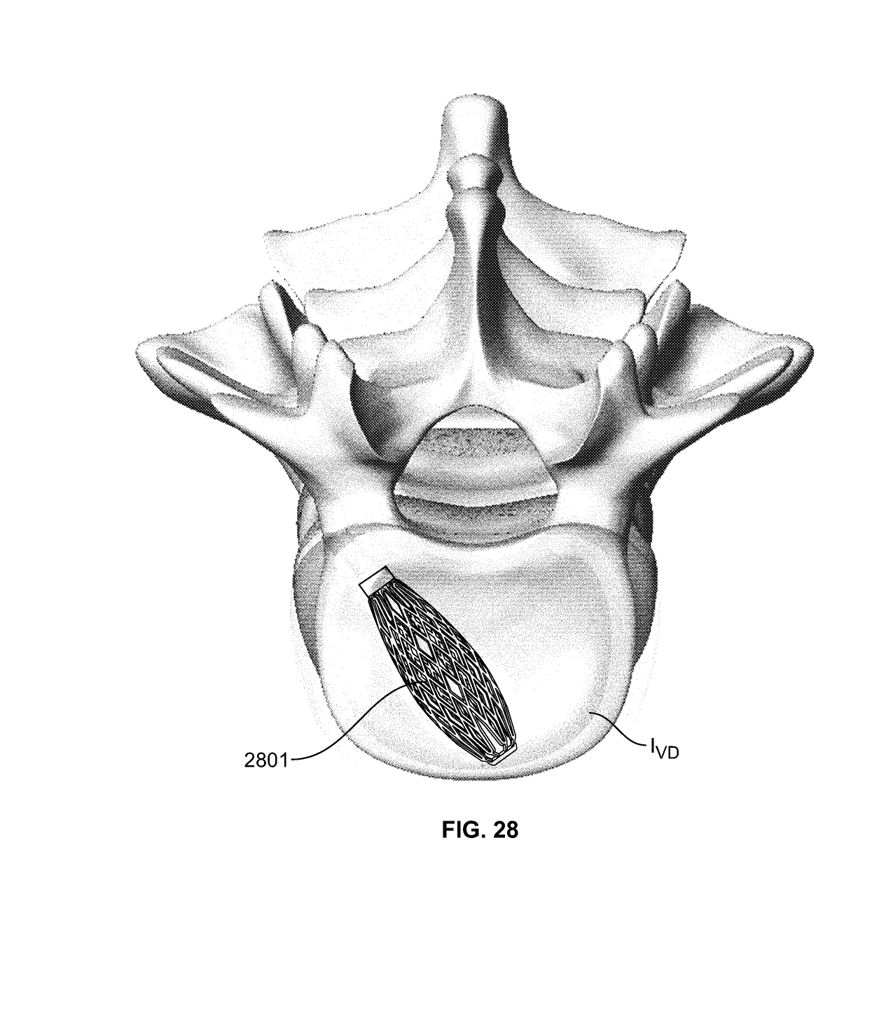

FIG. 28 shows an illustrative view of Ivd taken along cut lines 28-28, with V1 not being shown. The illustrative view shows cage 2801 expanded in Ivd. Cage 2801 may be cage 2501. Cage 2801 may be a cage different from cage 2501.

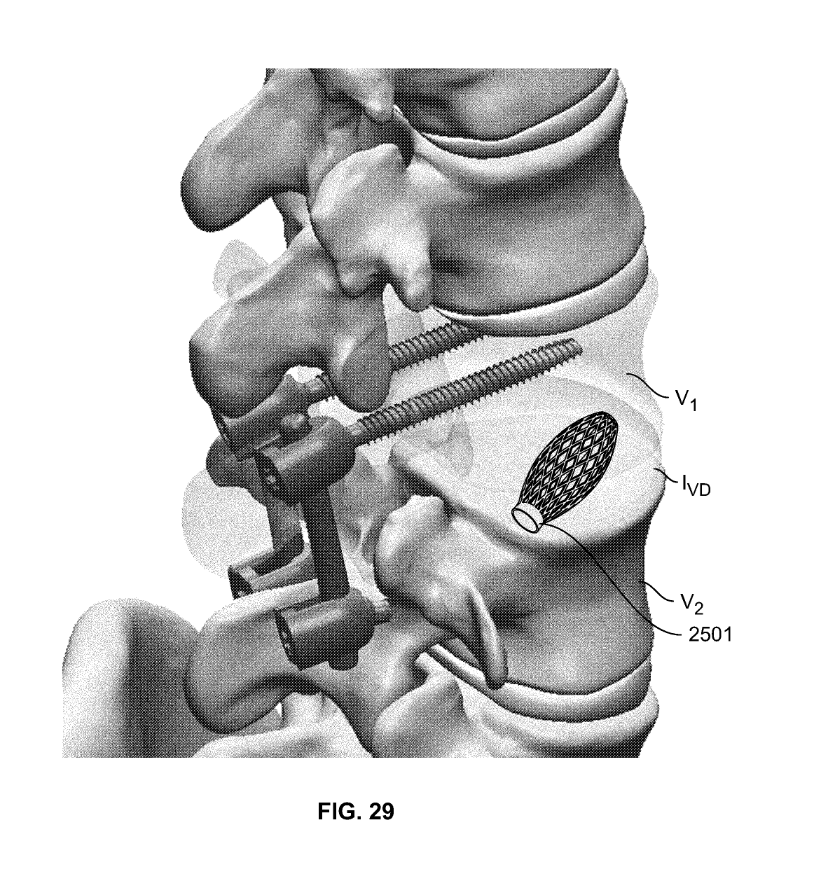

FIG. 29 shows external fixation of the augmented height of Ivd using bilateral pedicle screw and rods.

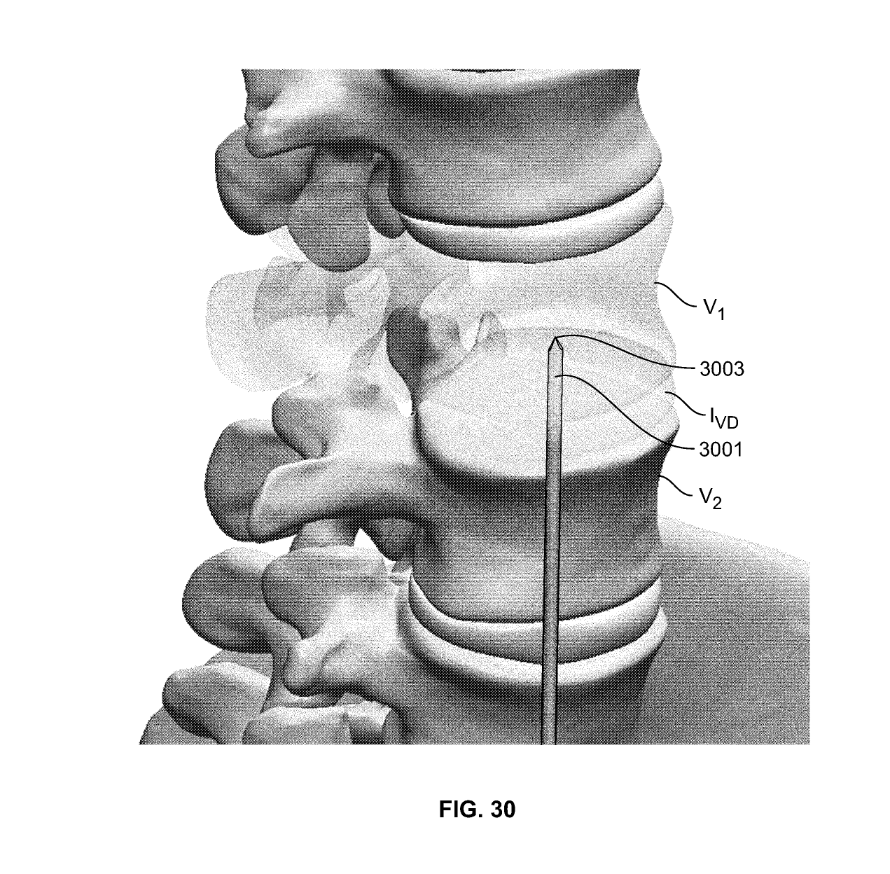

FIGS. 30-41 show an illustrative method for treating a spinal body. The spinal body may be intervertebral disc Ivd (“Ivd”). Ivd may be disposed between vertebra V1 and vertebra V2. The method illustrated in FIGS. 30-41 may include additional method steps disclosed herein. The method may be performed in the order shown. The method may be performed in an order different from the order shown. The method may involve one or more of some or all of the apparatus shown in FIGS. 30-41.

The method illustrated in FIGS. 30-41 is shown using a unilateral approach. The method shown in 30-41 may be performed using a bilateral approach.

In FIGS. 30-41, Ivd is accessed through a lateral side lateral side of Ivd. Ivd may be accessed through the lateral side during a Lateral Lumbar Interbody fusion and Transverse approach procedure. Such a procedure may also be referred to as Extreme Lateral Interbody Fusion and may be offered under the trademark “XLIF” available from Nuvasive, Inc. of San Diego, Calif.

FIG. 30 shows illustrative lateral placement of guide wire 3001 at target site 3003 in Ivd. Guide wire 3001 may be placed in Ivd at a desired trajectory and depth. Fluoroscopy may be used in both anterior-posterior and lateral planes to confirm placement. Guide wires may be inserted at multiple levels, or elevations, along the spine (not shown).

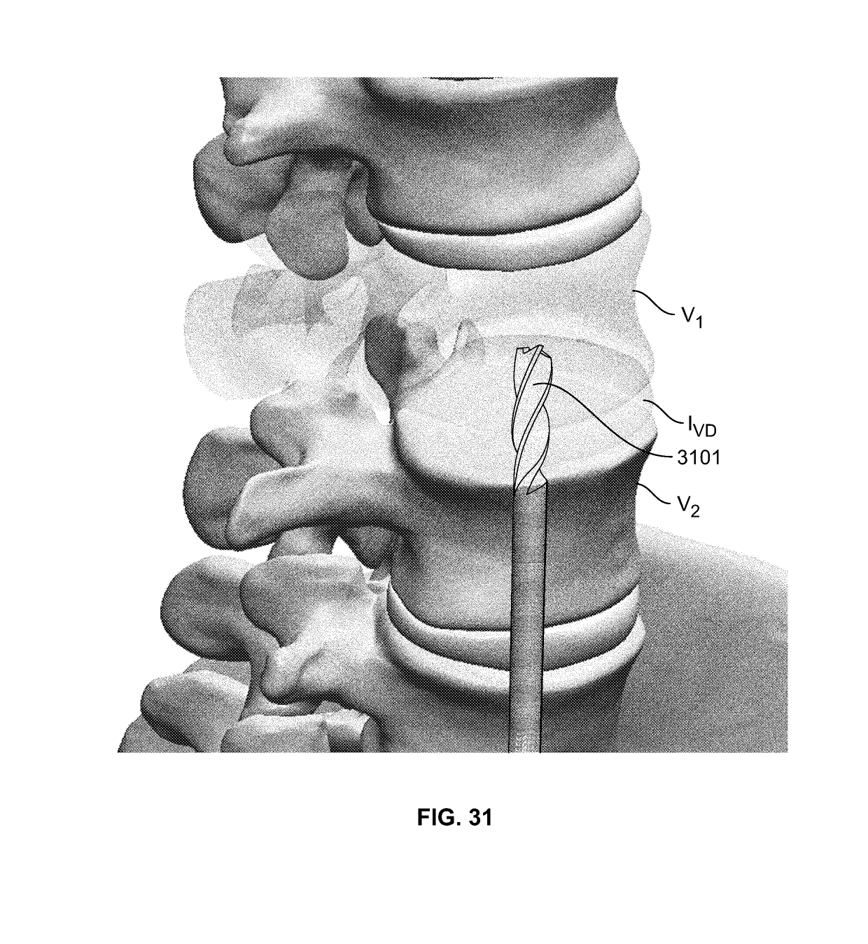

FIG. 31 shows illustrative drilling along an access path defined by guide wire 3001 to target site 3003. Drill 3101 may drill along the access path. Drill 3101 may be cannulated. Drill 3101 may be advanced along guide wire 3001 to target site 3003. Drill 3101 may be solid. Guide wire 3001 may be removed from Ivd prior to advancing drill 3101 along the access path and to target site 3002. The drilling may be performed to a depth in Ivd. The depth may be determined by using fluoroscopy and or instrumentation.

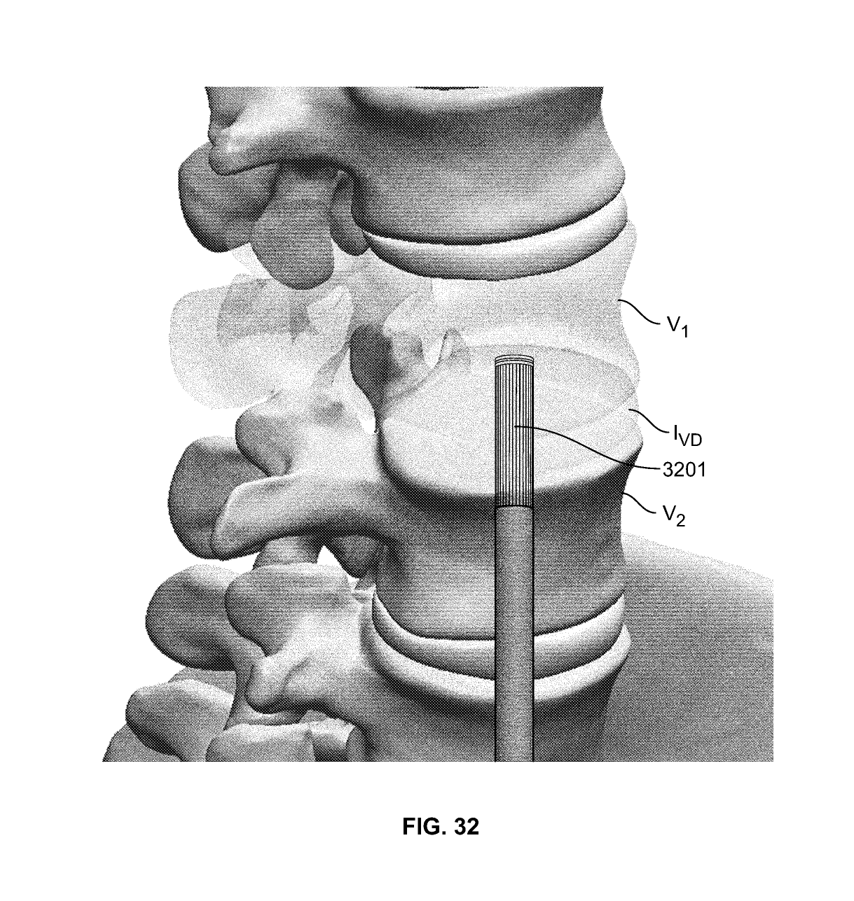

FIG. 32 shows illustrative placement of cage 3201 in Ivd. Cage 3201 is placed in Ivd in an unexpanded state. Cage 3201 may be advanced into Ivd through a diameter of a drill hole prepared by drill 3101.

Cage 3201 may have one or more features in common with the first cage. Cage 3201 may have one or more features in common with the second cage.

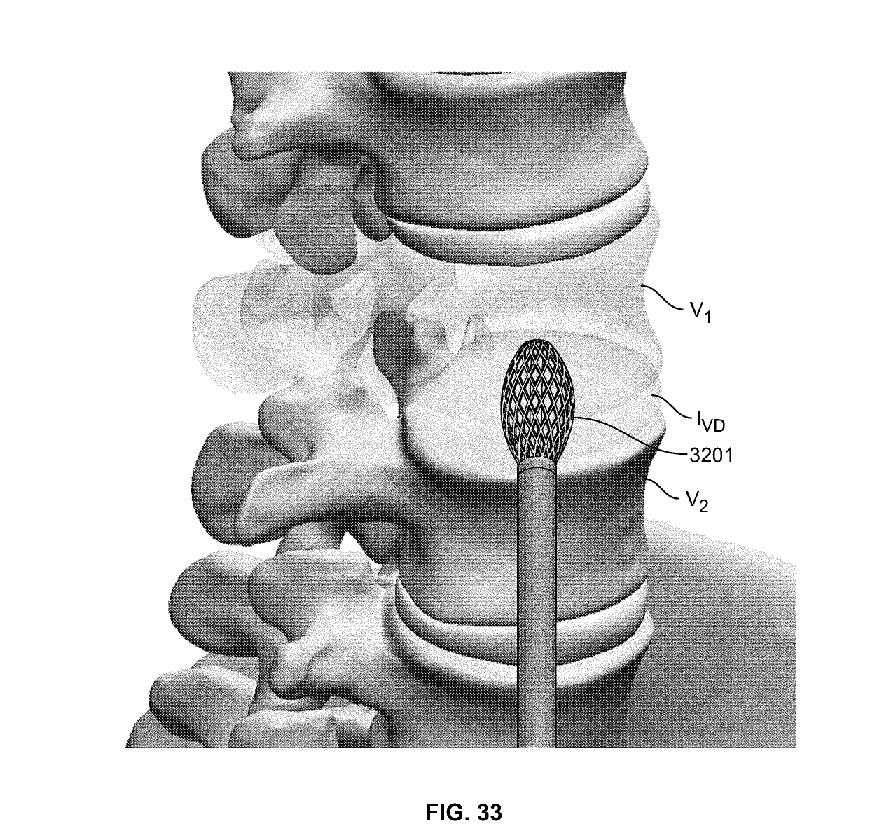

FIG. 33 shows illustrative augmenting of a height of Ivd (change in disc height not illustrated). The height of Ivd may be augmented by expanding cage 3201 in Ivd. Expanding cage 3201 may alleviate nerve compression or other pathology.

Cages 3201 may be an expandable mesh cage. Cage 3201 may be an implant. Cage 3201 may be formed from non-implantable material. Cage 3201 may be a distraction tool. Cage 3201 may be a cavity preparation instrument (not shown).

After augmentation of the height of Ivd, one or more pedicle screws may be advanced into one or both of V1 and V2. The pedicle screws may be provisionally tightened to hold the augmented height in place.

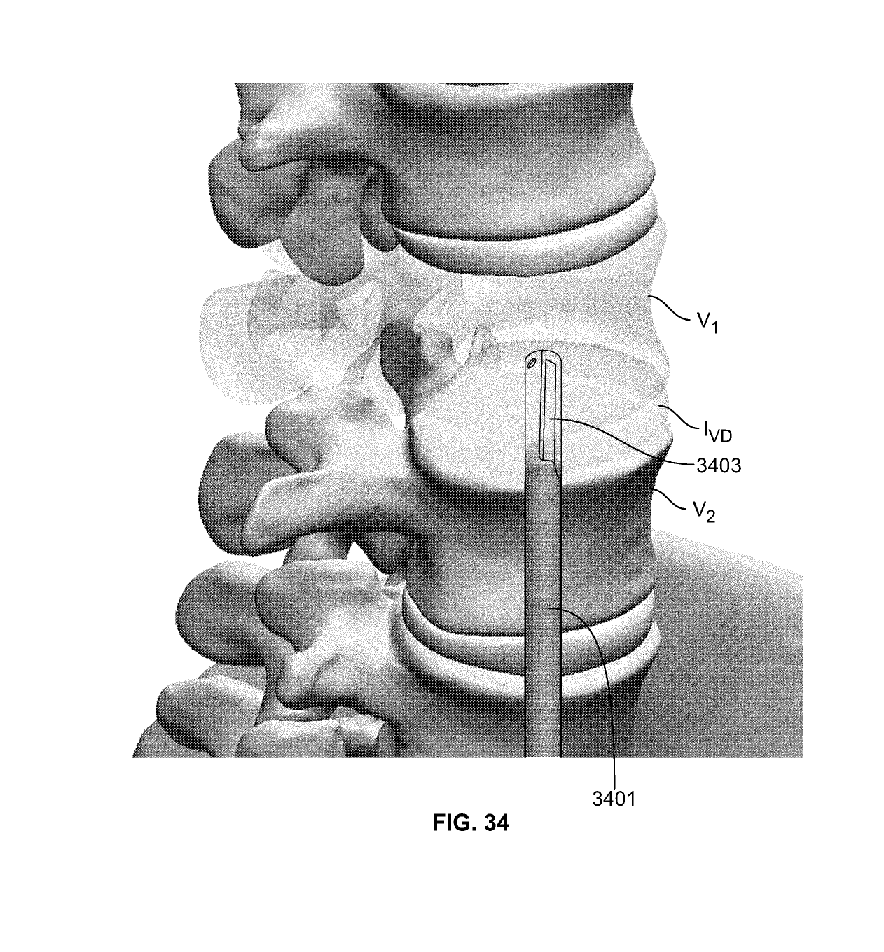

Cages 3201 may be removed from Ivd after augmenting the height of Ivd to a desired distraction. The method may proceed with the step illustrated in FIG. 34. Cage 3201 may be implanted in Ivd. External fixation mechanisms described herein may be used to maintain the augmented height of Ivd.

Cage 3201 may be used to prepare a cavity in Ivd. Cage 3201 may be circumferentially rotated to prepare the cavity. Cage 3201 may include one or more cutting edges.

FIG. 34 shows illustrative positioning cavity preparation instrument 3401 in Ivd. Broaching member 3403 is in a collapsed state. Cavity preparation instrument 3401 may be the cavity preparation instrument. Broaching member 3403 may be the broaching member.

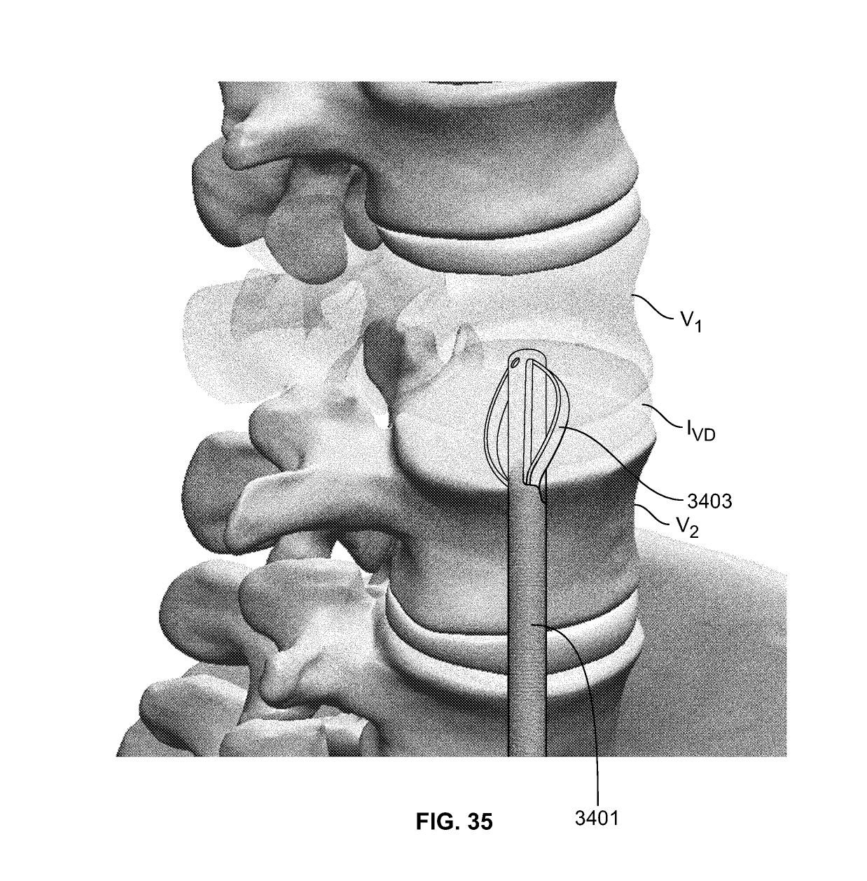

FIG. 35 shows illustrative preparation of a cavity in Ivd. In FIG. 35, broaching member 3403 has been activated. Rotating the broaching member in Ivd may displace material. Rotating the broaching member in Ivd may distract and remove disc material in Ivd. Fluoroscopy and tactile feedback may be used by a practitioner to judge a depth and size of disc dissection. Rotating broaching member in Ivd may prepare a vertebral end plate of V1 and a vertebral end plate of V2 for implantation of a cage. Broaching member may prepare the end plate for implantation by scraping the end plates and thereby promoting bleeding bone.

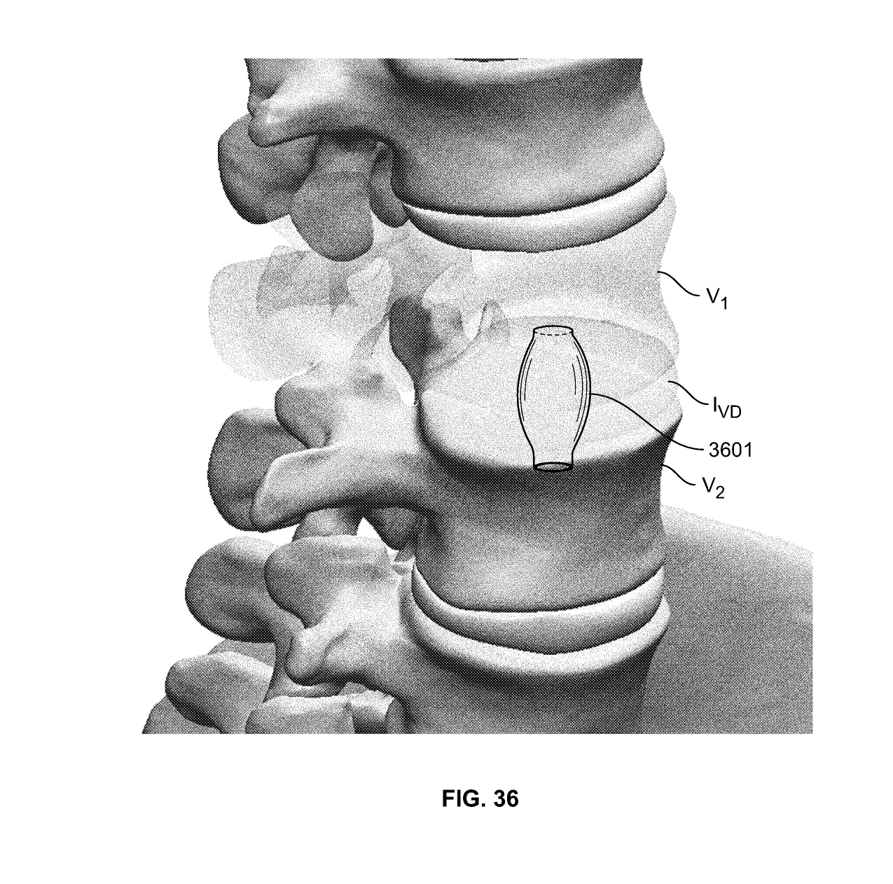

FIG. 36 shows an illustrative view of cavity 3601. Cavity 3601 may be prepared by cavity preparation instrument 3401.

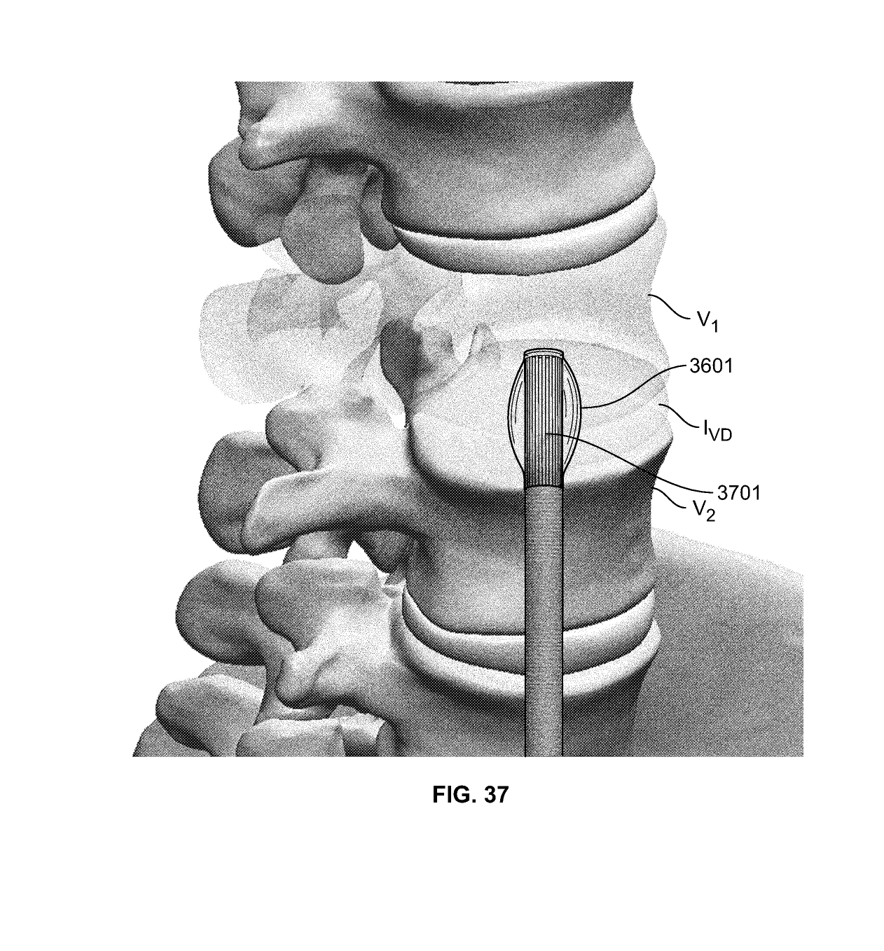

FIG. 37 shows illustrative positioning of cage 3701, in an unexpanded state, in cavity 3601. Cage 3701 may have one or more features in common with the first cage. Cage 3701 may have one or more features in common with the second cage.

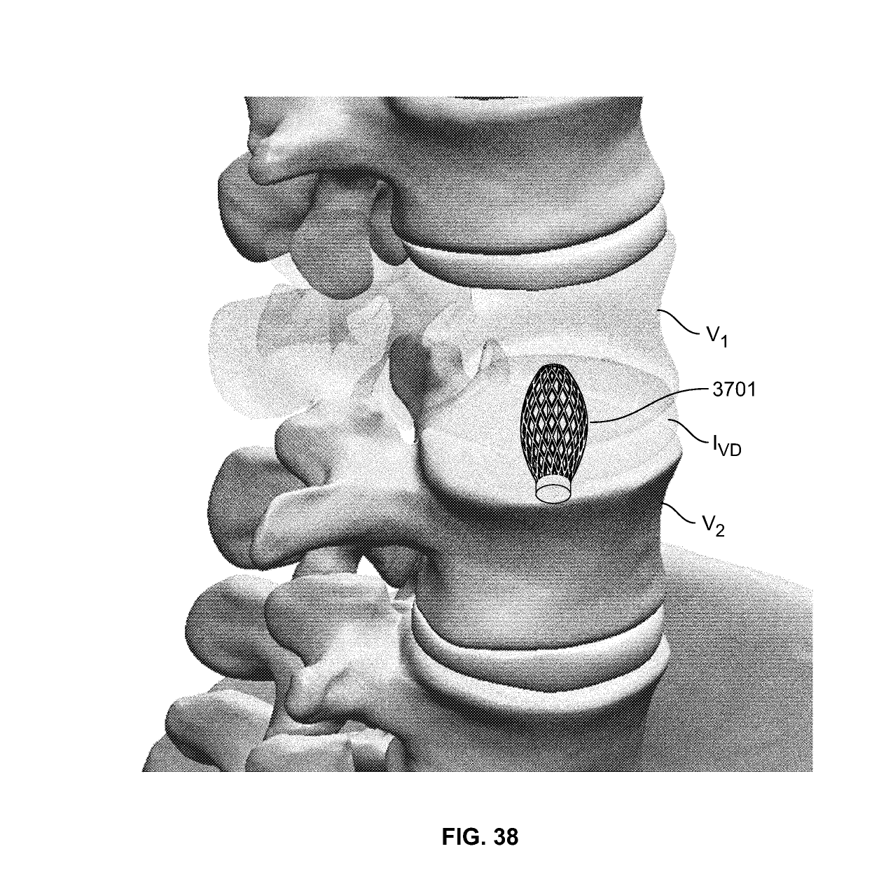

FIG. 38 shows illustrative cage 3701, in an expanded state, positioned in Ivd with a first handle of the cage having been removed.

A second handle may be coupled to cage 3701. The second handle may be used to rotate cage 3701. Rotating cage 3701 may further expand the cage 3701. Rotating cage 3701 may pack cage 3701 with disc material. Rotating cage 3701 may further conform a contour of the end plates to a contour of an implant.

An interior of cage 3701 may be packed with material such as displaced bone, bone graft, and bone cement. The material may be packed in the interior prior to positioning the cage in Ivd. The material may be packed in the interior after positioning the cage in Ivd.

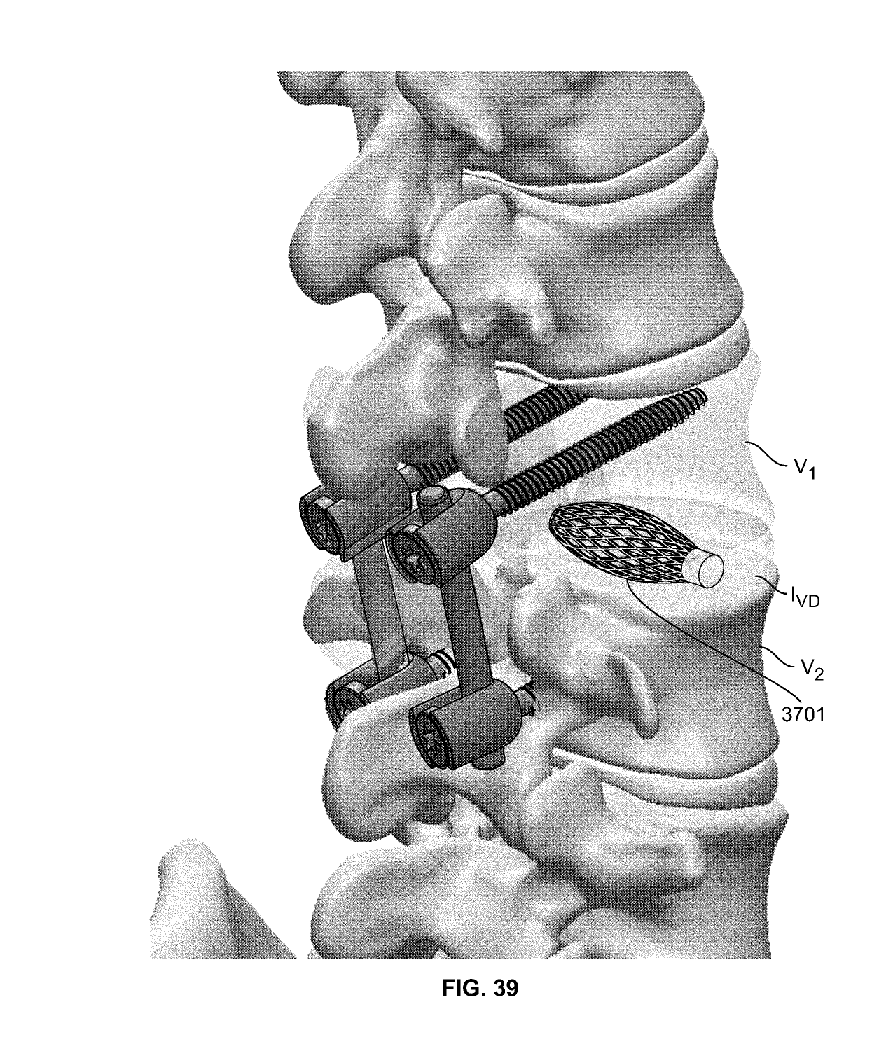

FIG. 39 shows illustrative supplemental fixation of V1 and V2. The supplemental fixation may include driving pedicle screws into V1. The supplemental fixation may include driving pedicle screws into V2. The supplemental fixation illustrated is bilateral. Alternatively, supplemental fixation of V1 and V2 may be unilateral. A rod may pass through two of the screws.

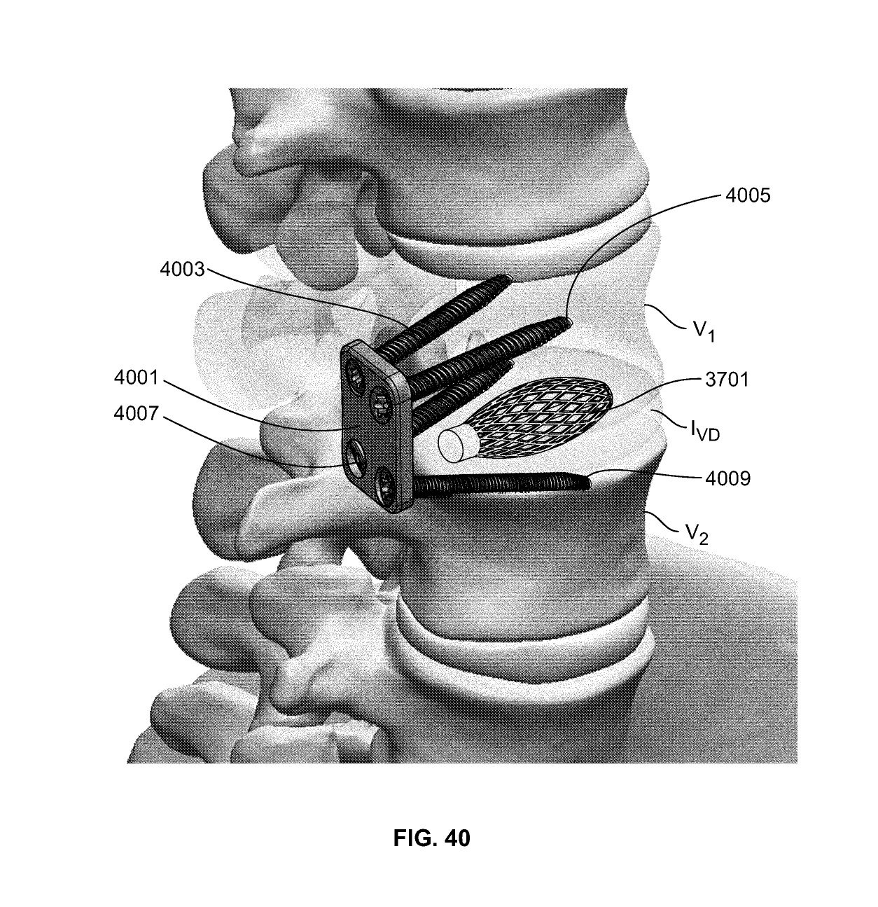

FIG. 40 shows illustrative supplemental fixation of V1 and V2. The supplemental fixation may include plate 4001. Plate 4001 may receive screws 4003, 4005, 4007 and 4009. A width of screws 4003, 4005, 4007 and 4009 is illustrative. The screws may have a with similar to the width illustrated in FIG. 39.

Plate 4001 may provide supplemental fixation to V1 and V1. The size of plate 4001 is illustrative. An appropriate size plate and length screws may be chosen to fit the anatomy of patient. The placement of the plate may be chosen to fit the anatomy of the patient.

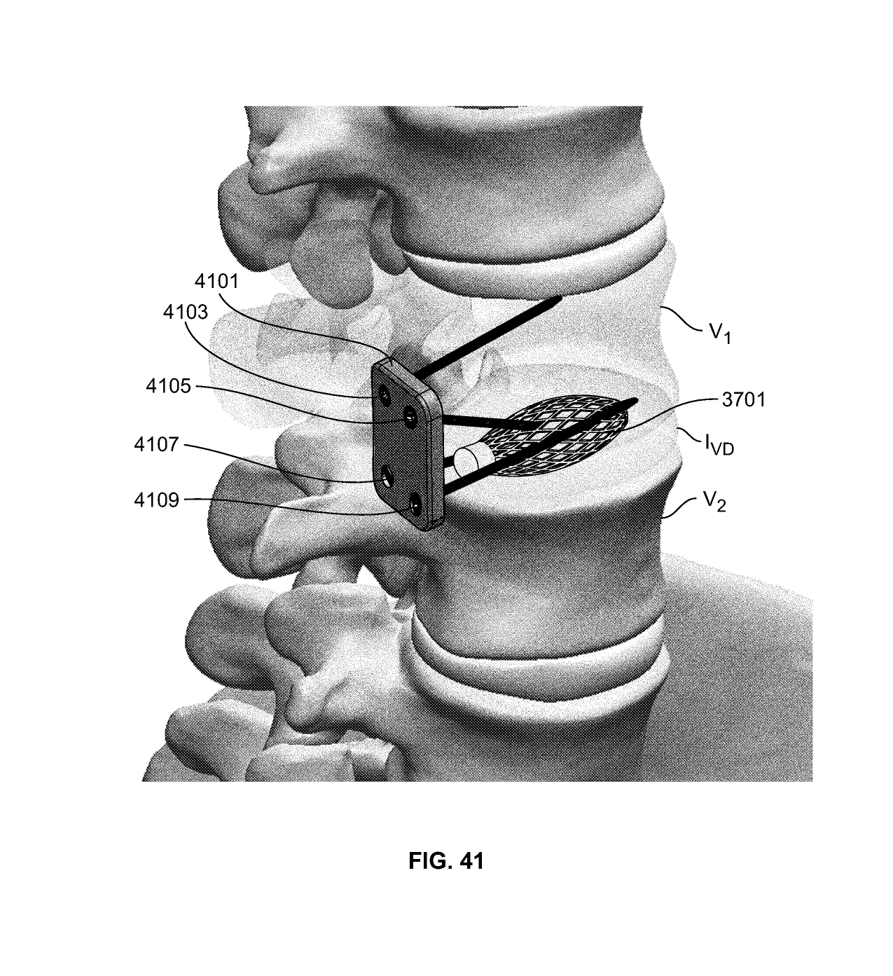

FIG. 41 shows illustrative supplemental fixation of V1 and V2. The supplemental fixation may include plate 4101. Plate 4010 may be positioned on V1. Plate 4010 may receive screws 4103, 4105, 4107, and 4109.

Cage 3701 may include a plurality of cells. Screw 4109 may be sized to pass through the cells of cage 3701. Screw 4109 is illustrated passing through two cells of cage 3701. Screw 4109 may be cannulated. A fixation element may be advanced through plate 4101 and through the two cells. Screw 4109, when cannulated, may be advanced along the fixation element and through the two cells.

Screw 4105 may be sized to pass through the cells of cage 3701. Screw 4105 is illustrated passing through a cell of cage 3701. Screw 4105 may be cannulated. A fixation element may be advanced through plate 4101 and through the cell. Screw 4105, when cannulated, may be advanced along the fixation element and through the cell.

Screws 4103 and 4107 are illustrated as not passing through one or two cells of cage 3701. One or both of screws 4103 and screws 4107 may pass through one or two cells of cage 3701.

When screws 4103 and 4107 do not pass through a cell of cage 3701, screws 4103 and 4107 may be sized larger than a cell of cage 3701. For example, screws 4103 and 4107 may have a diameter similar to the diameter of screw 3905 illustrated in FIG. 39. When screws 4103 and 4107 do not pass through a cell of cage 3701, screws 4103 and 4107 may be sized to pass through a cell of cage 3701.

A plate in accordance with the invention may define a first opening and a second opening. The first opening may receive a first screw. The first screw may engage a cell of a cage. The second opening may receive a second screw. The second screw may not engage a cell of a cage. The first opening may be smaller than the second opening.

Advancing one or more screws through cage 3701 may provide increased stiffness to the mesh of cage 3701.

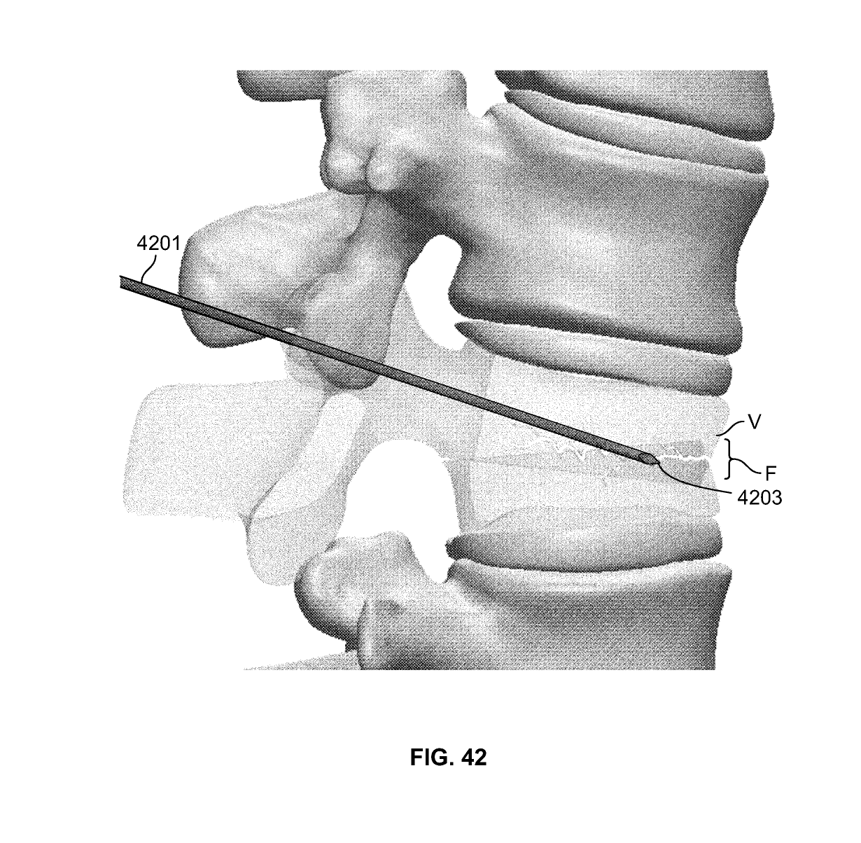

FIGS. 42-48 show an illustrative method for treating a spinal body. The spinal body may be vertebra V. Vertebra V may include an interior. Vertebra may have fracture site F.

The method illustrated in FIGS. 42-48 may include additional method steps disclosed herein. The method may be performed in the order shown. The method may be performed in an order different from the order shown. The method may involve one or more of some or all of the apparatus shown in FIGS. 42-48.

The method shown in FIGS. 42-48 may be an illustrative method for kyphoplasty. The method shown in FIGS. 42-48 is illustrated as unilateral, lumbar, transpedicular approach. Access to the interior is made through the pedicle.

The method shown in FIGS. 42-48 may be performed in a thoracic vertebra. The method shown in FIGS. 42-48 may be performed in any vertebra in the spine.

The method shown in FIGS. 42-48 may be performed using an extrapedicular approach. The method shown in FIGS. 42-48 may be performed using a transpedicular approach. The method shown may be using a extrapedicular and a transpedicular approach. The method shown in FIGS. 42-48 may be performed using a bilateral approach.

In FIGS. 42-48, vertebra V is accessed through a posterior face of vertebra V.

FIG. 42 shows illustrative posterior placement of guide wire 4201 at target site 4203 in vertebra V. Guide wire 4201 may be placed in vertebra V at a desired trajectory and depth. Fluoroscopy may be used in both anterior-posterior and lateral planes to confirm placement. Guide wires may be inserted at multiple levels, or elevations, along the spine (not shown).

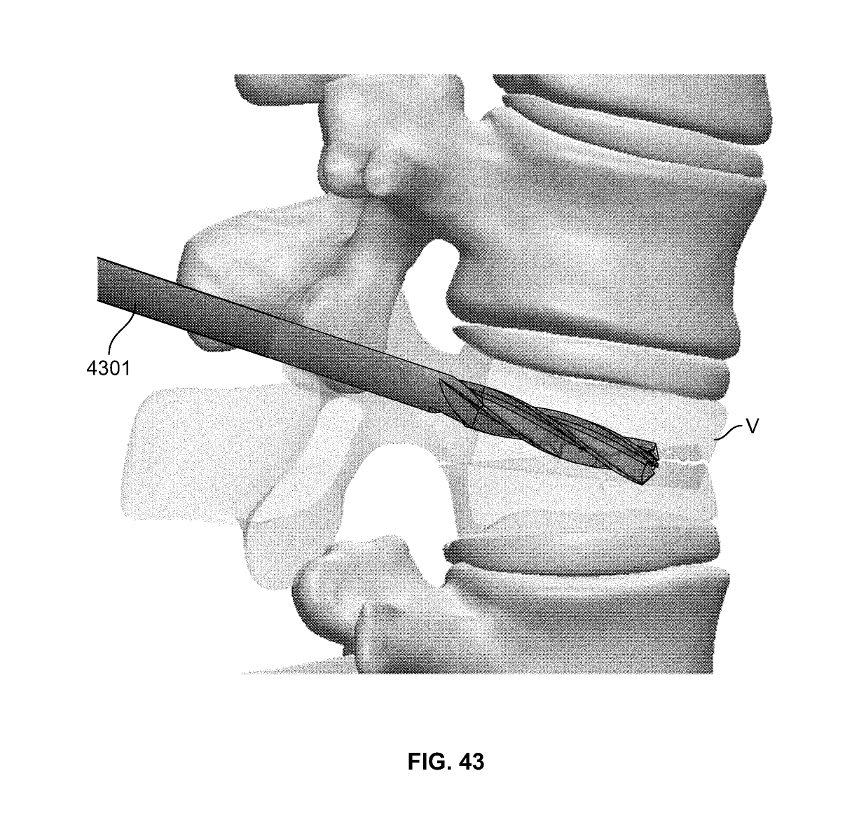

FIG. 43 shows illustrative drilling along an access path defined by guide wire 4201 to target site 4203. Drill 4301 may drill along the access path. Drill 4301 may be cannulated. Drill 4301 may be advanced along guide wire 4201 to target site 4203. Drill 4301 may be solid. Guide wire 4201 may be removed from vertebra V prior to advancing drill 4301 along the access path and to target site 4203. The drilling may be performed to a depth in vertebra V. The depth may be determined by using fluoroscopy and or instrumentation.

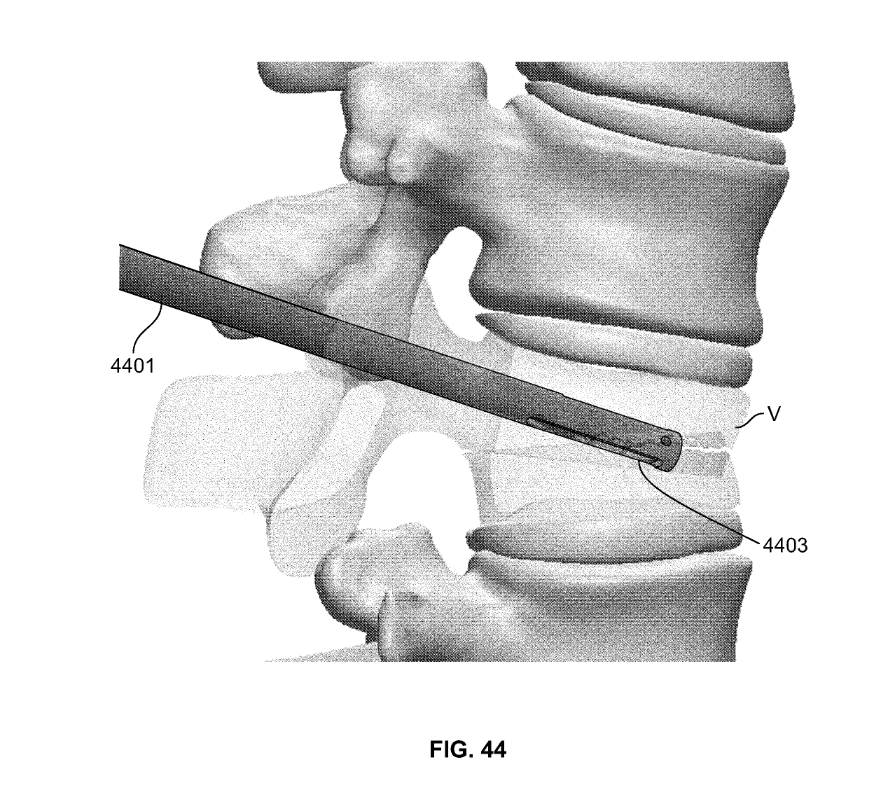

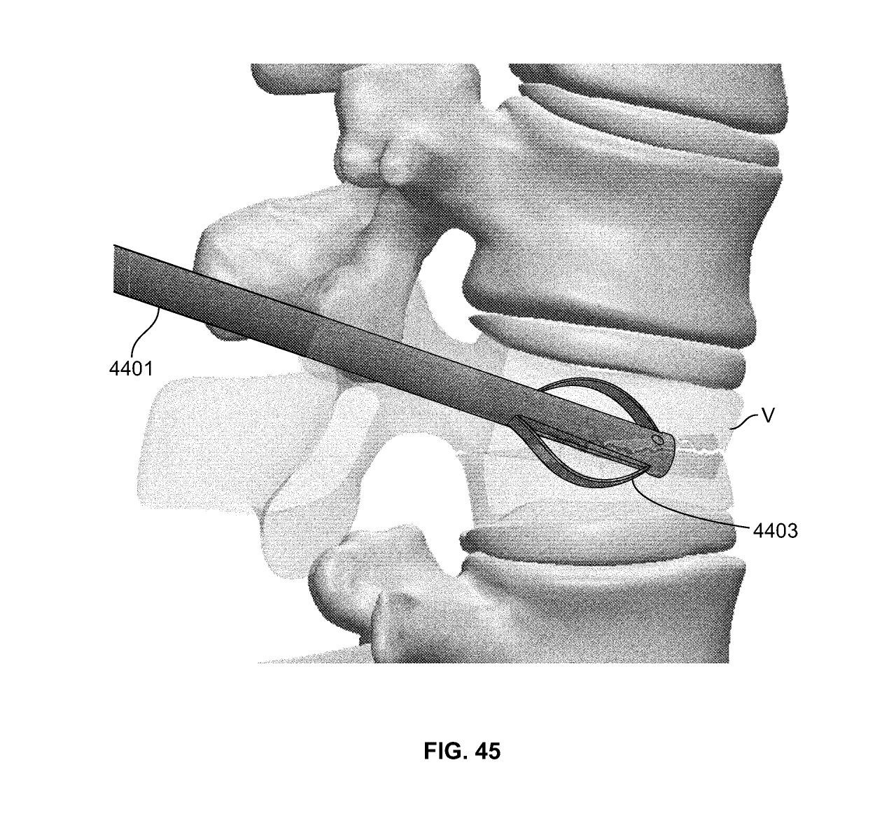

FIG. 44 shows illustrative positioning of cavity preparation instrument 4401 in vertebra V. Broaching member 4403 is shown in a collapsed state. Cavity preparation instrument 4401 may be the cavity preparation instrument. Broaching member 4403 may be the broaching member.

FIG. 45 shows illustrative preparation of a cavity in vertebra V. In FIG. 45, broaching member 4403 has been activated. Rotating broaching member 4403 in vertebra V may displace bone material. Rotating broaching member 4403 in vertebra V may distract and remove bone material in vertebra V. Fluoroscopy and tactile feedback may be used by a practitioner to judge a depth and size of bone displacement.

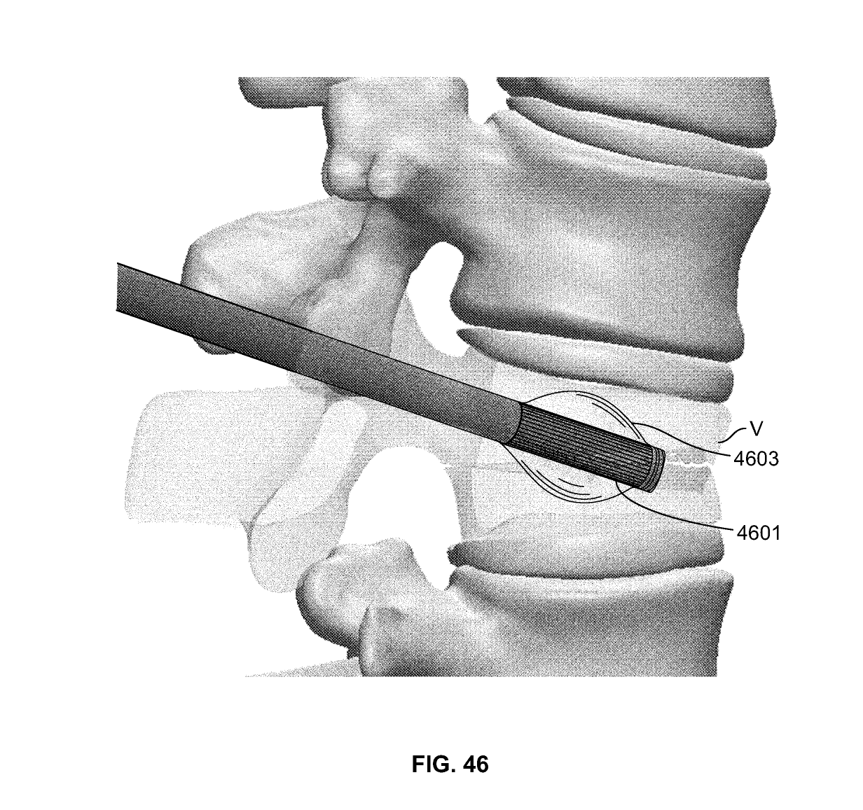

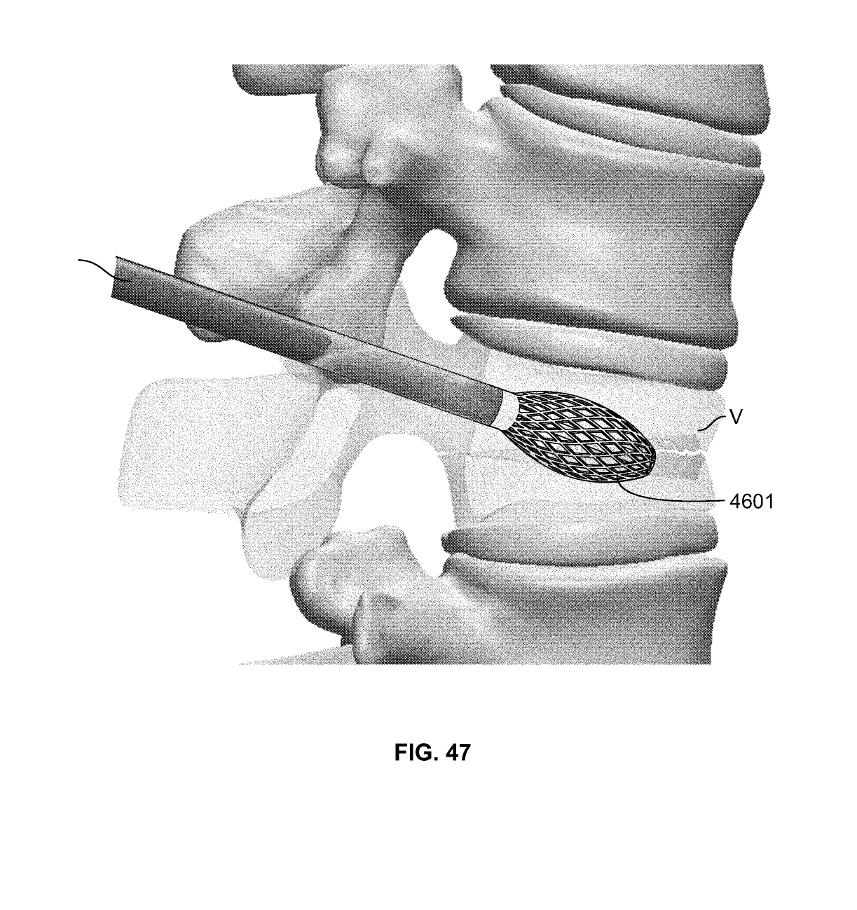

The method may continue at FIG. 46. A cavity formed by broaching member 4403 may be filled with bone cement, bone graft or any other suitable material. The material deposited in the cavity may augment a height of vertebra V. The material deposited in the cavity may not augment a height of vertebra V. The material deposited in the cavity may promote bone growth. After filling the cavity, an incision used to access vertebra V may be surgically closed.

FIG. 46 shows illustrative positioning of cage 4601, in an unexpanded state, in cavity 4603. Cavity 4603 may be prepared by cavity preparation instrument 4401. The method step illustrated in FIG. 46 may include positioning an inserter into the cavity (not shown). An inserter may be an instrument used to deliver material into a cavity such as cavity 4603. The inserter may be disposed within cage 4601. The inserter may be disposed on an outer face of cage 4601.

Cage 4601 may have one or more features in common with the first cage. Cavity 4601 may have one or more features in common with the second cage.

FIG. 47 shows illustrative expansion of cage 4601. Cage 4601 may be expanded in cavity 4603 to fill cavity 4603. Expansion of cage 4601 in vertebra V may not augment a height of vertebra V. Expansion of cage 4601 in vertebra V may augment a height of vertebra V.

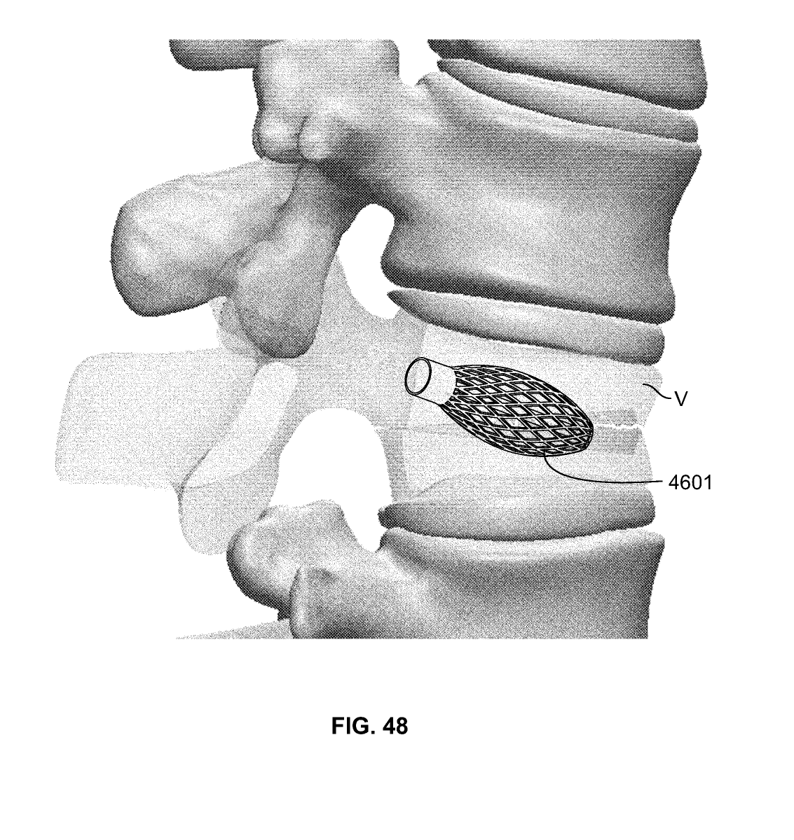

FIG. 48 shows illustrative cage 4601 implanted in vertebra V after a handle of cage 4601 has been removed. After cage 4601 is implanted in vertebra V, cage 4601 may be filled with bone cement, bone great, or any other suitable material. When an inserter is used to fill cage 4601, the inserter may be removed after cage 4601 has been filled. Removing the inserter may leave behind the material deposited in cage 4601.

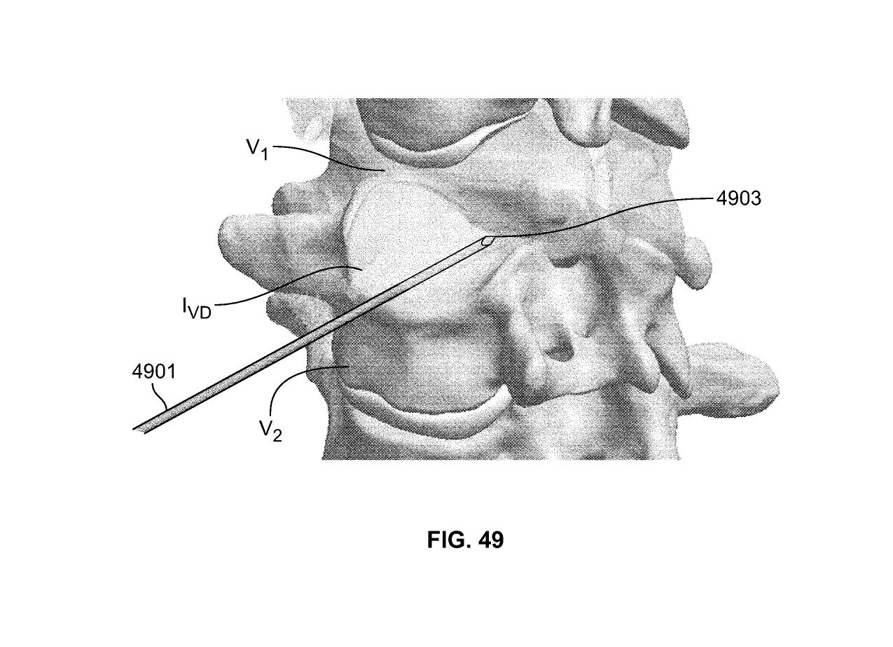

FIGS. 49-60 show an illustrative method for treating a spinal body. The spinal body may be intervertebral disc Ivd (“Ivd”). Ivd may be disposed between vertebra V1 and vertebra V2. The method illustrated in FIGS. 49-60 may include additional method steps disclosed herein. The method may be performed in the order shown. The method may be performed in an order different from the order shown. The method may involve one or more of some or all of the apparatus shown in FIGS. 49-60.

In FIGS. 49-60, Ivd is accessed through a lateral side of Ivd. Ivd may be accessed through a lateral side during an Anterior Cervical Discectomy Fusion (“ACDF”) procedure.

FIG. 49 shows illustrative lateral placement of guide wire 4901 at target site 4103 in Ivd. Guide wire 4901 may be placed in Ivd at a desired trajectory and depth. Fluoroscopy may be used in both anterior-posterior and lateral planes to confirm placement. Guide wires may be inserted at multiple levels, or elevations, along the spine (not shown).

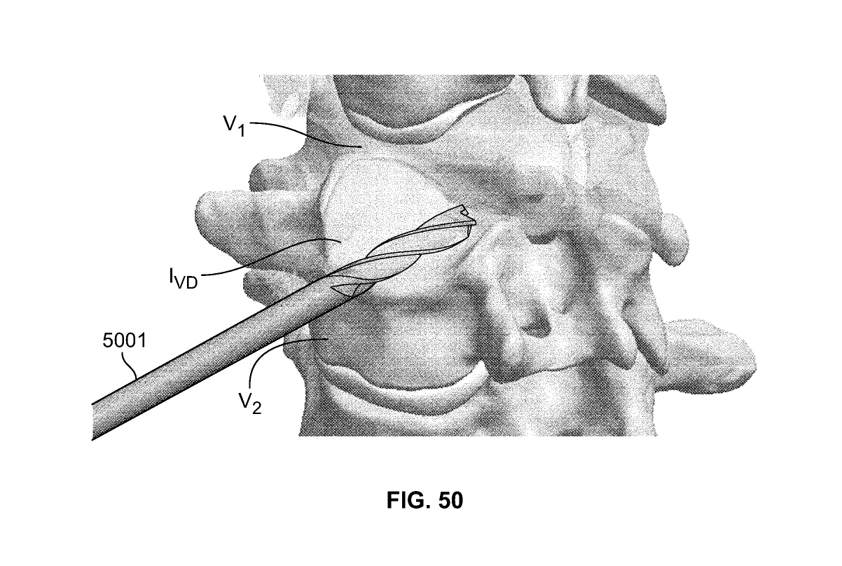

FIG. 50 shows illustrative drilling along an access path defined by guide wire 4901 to target site 4903. Drill 5001 may drill along the access path. Drill 5001 may be cannulated. Drill 5001 may be advanced along guide wire 4901 to target site 4903. Drill 5001 may be solid. Guide wire 4901 may be removed from Ivd prior to advancing drill 5001 along the access path. The drilling may be performed to a depth in Ivd. The depth may be determined by using fluoroscopy and or instrumentation.

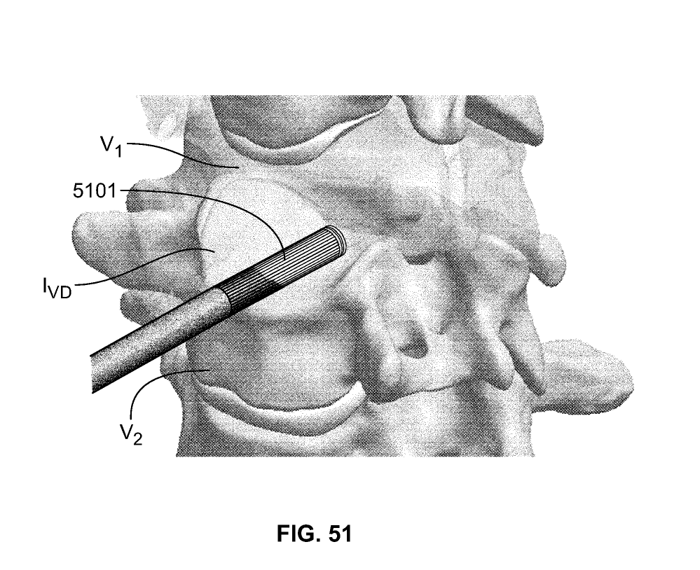

FIG. 51 shows illustrative placement of cage 5101 in Ivd. Cage 5101 may be placed in Ivd in an unexpanded state. Cage 5101 may be advanced into Ivd through a diameter of a drill hole prepared by drill 5001.

Cage 5101 may have one or more features in common with the first cage. Cage 5101 may have one or more features in common with the second cage.

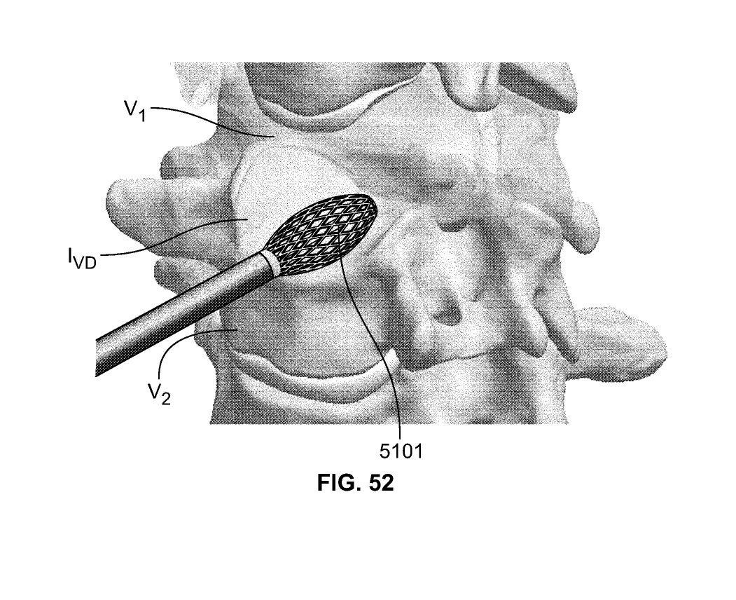

FIG. 52 shows illustrative augmenting of a height of Ivd (change in disc height not illustrated). The height of Ivd may be augmented by expanding cage 5101 in Ivd. Expanding cage 5101 may alleviate nerve compression or other pathology.

Cage 5101 may be an expandable mesh cage. Cage 5101 may be an implant. Cage 5101 may be formed from non-implantable material. Cage 5101 may be a distraction tool. Cage 5101 may be the cavity preparation instrument (not shown).

After augmentation of the height of Ivd, one or more pedicle screws may be advanced into one or both of V1 and V2. The pedicle screws may be provisionally tightened to hold the augmented height in place.

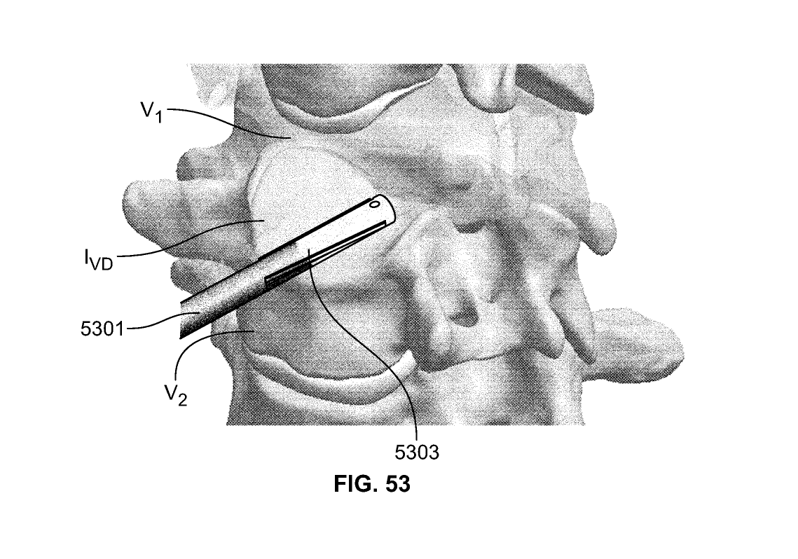

Cages 5101 may be removed from Ivd after augmenting the height of Ivd to a desired distraction. The method may proceed with the step illustrated in FIG. 53. Cage 5101 may be implanted in Ivd. External fixation mechanisms described herein may be used to maintain the augmented height of Ivd.

Cage 5101 may be used to prepare a cavity in Ivd. Cage 5101 may be circumferentially rotated to prepare the cavity. Cage 5101 may include one or more cutting edges.

FIG. 53 shows illustrative positioning of cavity preparation instrument 5301 in Ivd. Broaching member 5303 is shown in a collapsed state.

Cavity preparation instrument 5301 may be the cavity preparation instrument. Broaching member 5303 may be the broaching member.

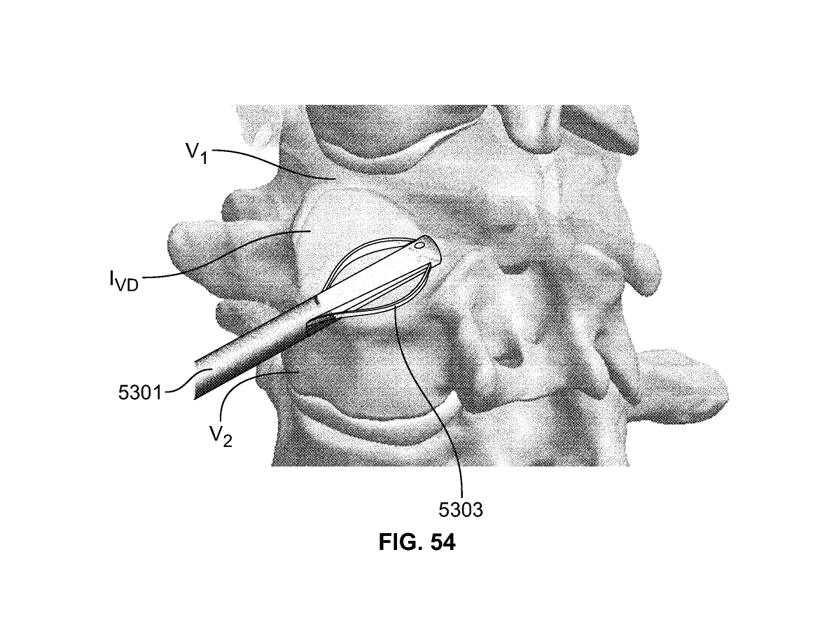

FIG. 54 shows illustrative preparation of a cavity in Ivd. In FIG. 54, broaching member 5303 has been activated. Rotating the broaching member in Ivd may cut bone material. Rotating the broaching member in Ivd may distract and remove disc material in Ivd. Fluoroscopy and tactile feedback may be used by a practitioner to judge a depth and size of disc dissection. Rotating broaching member in Ivd may prepare a vertebral end plate of V1 and a vertebral end plate of V2 for implantation of a cage. Broaching member may prepare the end plate for implantation by scraping the end plates and thereby promoting bleeding bone.

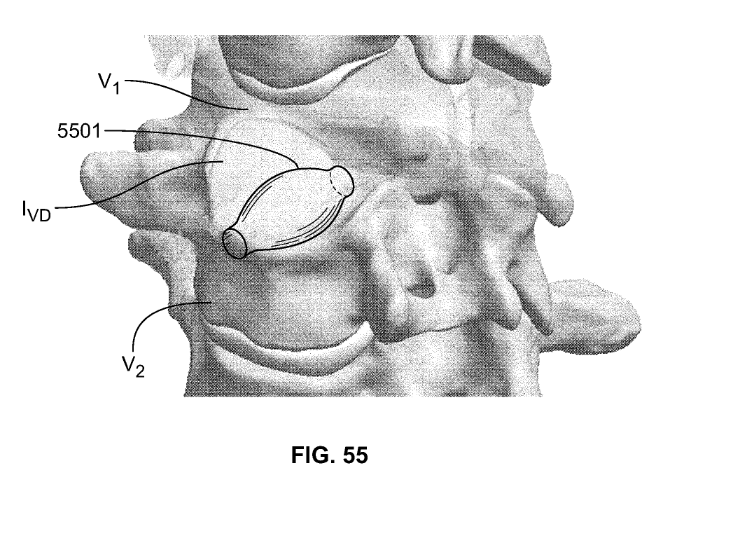

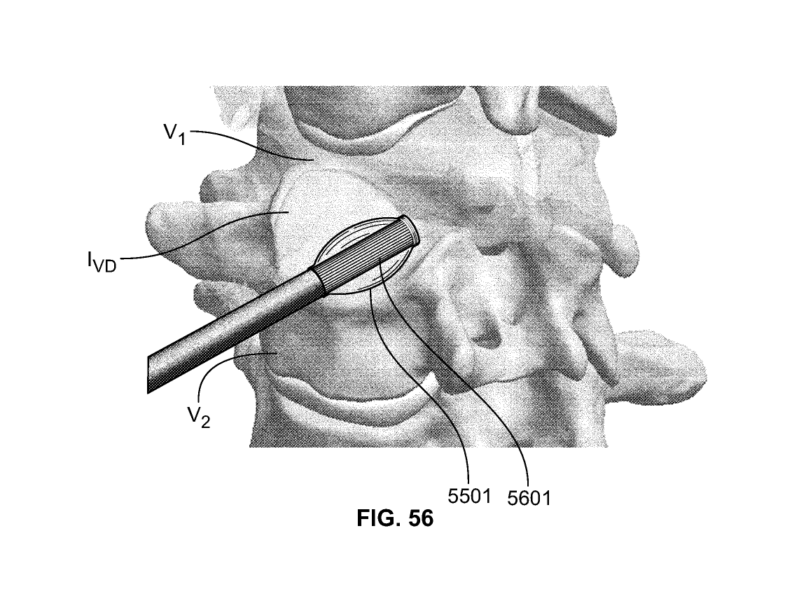

FIG. 55 shows an illustrative view of cavity 5501. Cavity 5501 may be prepared by cavity preparation instrument 5301.

FIG. 56 shows illustrative positioning of cage 5601, in an unexpanded state, in cavity 5501. Cage 5601 may have one or more features in common with the first cage. Cage 5601 may have one or more features in common with the second cage.

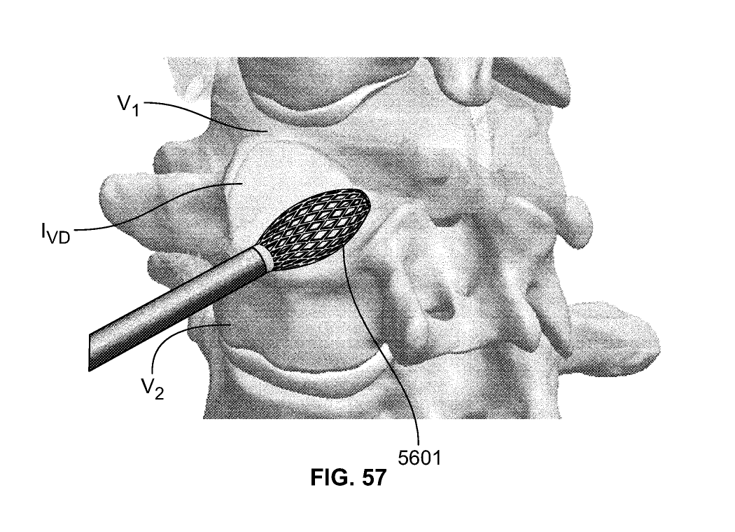

FIG. 57 shows illustrative expansion of cage 5601 in cavity 5501. Cage 5601 may be expanded to fill cavity 5501.

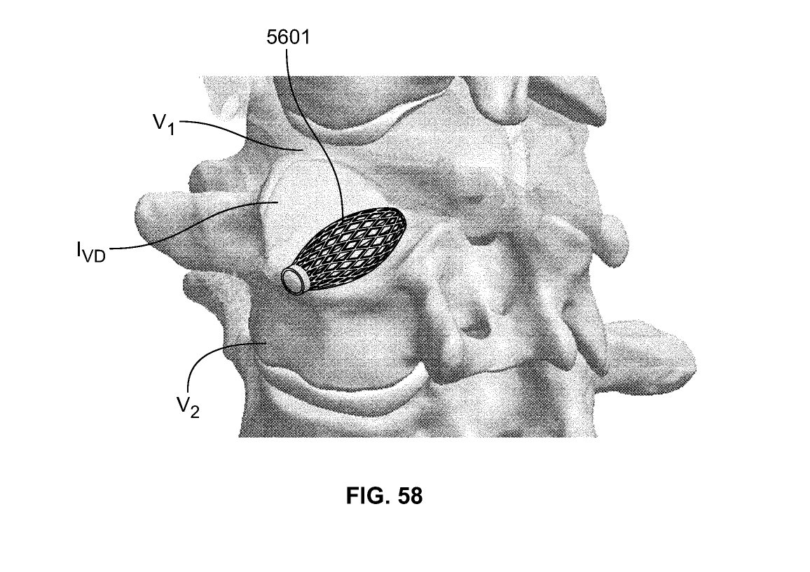

FIG. 58 shows cage 5601 in Ivd after a handle of cage 5601 has been removed. Cage 5601 may be filled with material as described at FIG. 48.

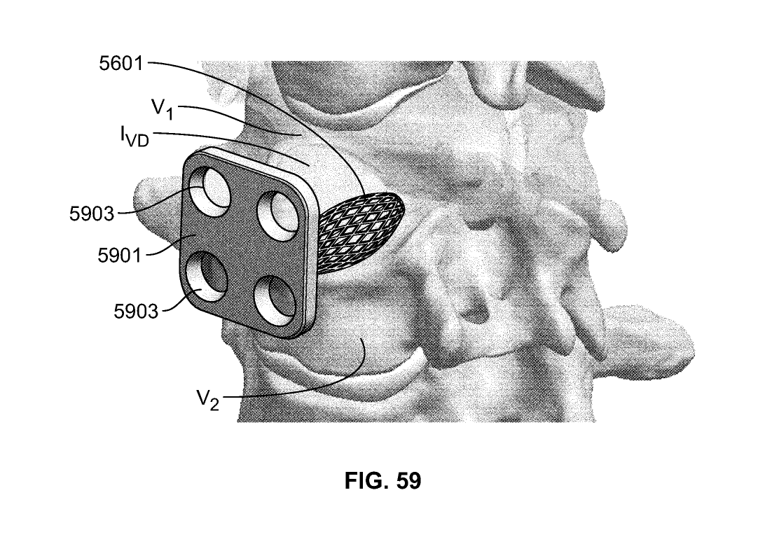

FIG. 59 shows plate 5901 positioned on a lateral face of Ivd. Plate 5901 may extend along V1, Ivd and V2. Plate 5901 defines holes 5903. Holes 5903 are sized for receiving screws.

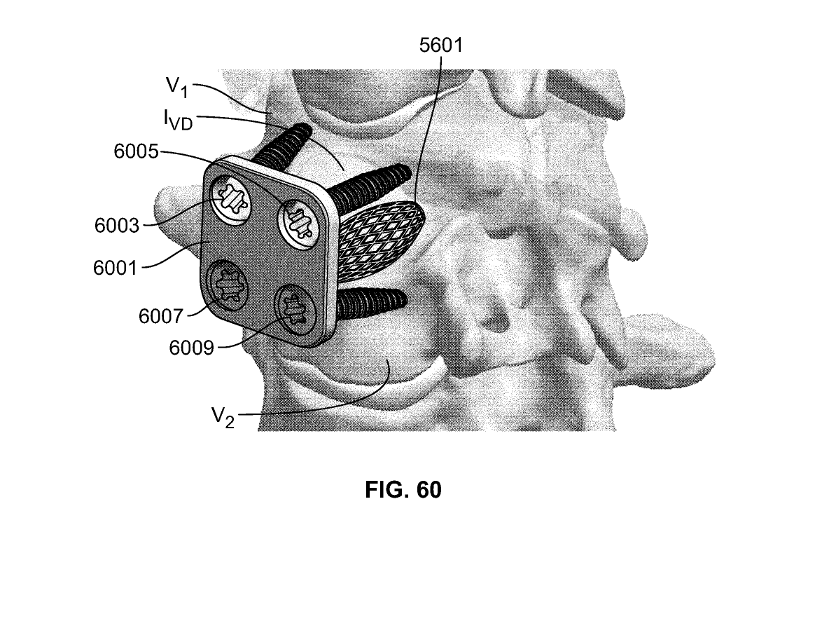

FIG. 60 shows plate 6001 positioned on a lateral face of Ivd. Plate 6001 may extend along V1, Ivd and V2. Each of screws 6003, 6005, 6007 and 6009 are engaged with a hole defined by plate 6001. Screws 6003, 6005, 6007 and 6009 may not contact cage 5601.

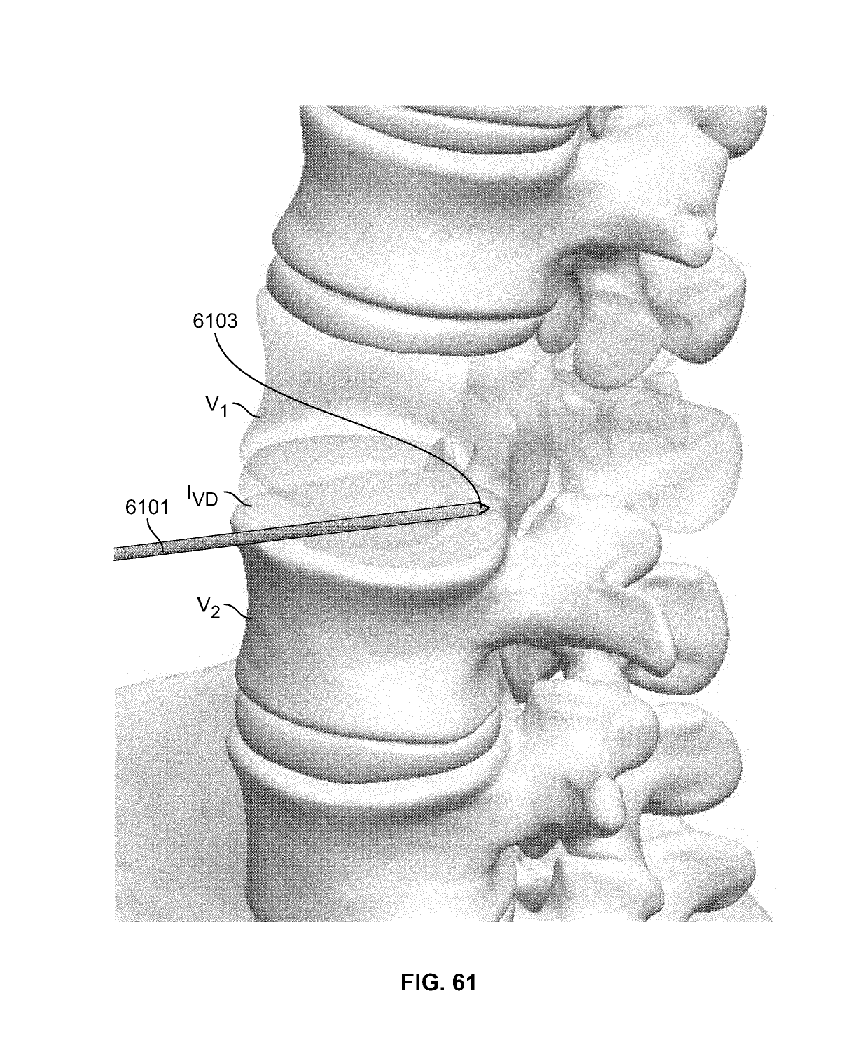

FIGS. 61-69 show an illustrative method for treating a spinal body. The spinal body may be intervertebral disc Ivd (“Ivd”). Ivd may be disposed between vertebra V1 and vertebra V2. The method illustrated in FIGS. 61-69 may include additional method steps disclosed herein. The method may be performed in the order shown. The method may be performed in an order different from the order shown. The method may involve one or more of some or all of the apparatus shown in FIGS. 61-69.

In FIGS. 61-69, Ivd is accessed through an anterior side of Ivd. Ivd may be accessed through an anterior side during an Anterior Lateral Interbody Fusion (“ALIF”) procedure.

FIG. 61 shows illustrative anterior placement of guide wire 6101 in Ivd. A tip of guide wire 6101 may be positioned at target site 6103. Guide wire 6101 may be inserted into Ivd at a desired trajectory and depth. Fluoroscopy may be used in an anterior-posterior plate. Fluoroscopy may be used in one or both lateral planes.

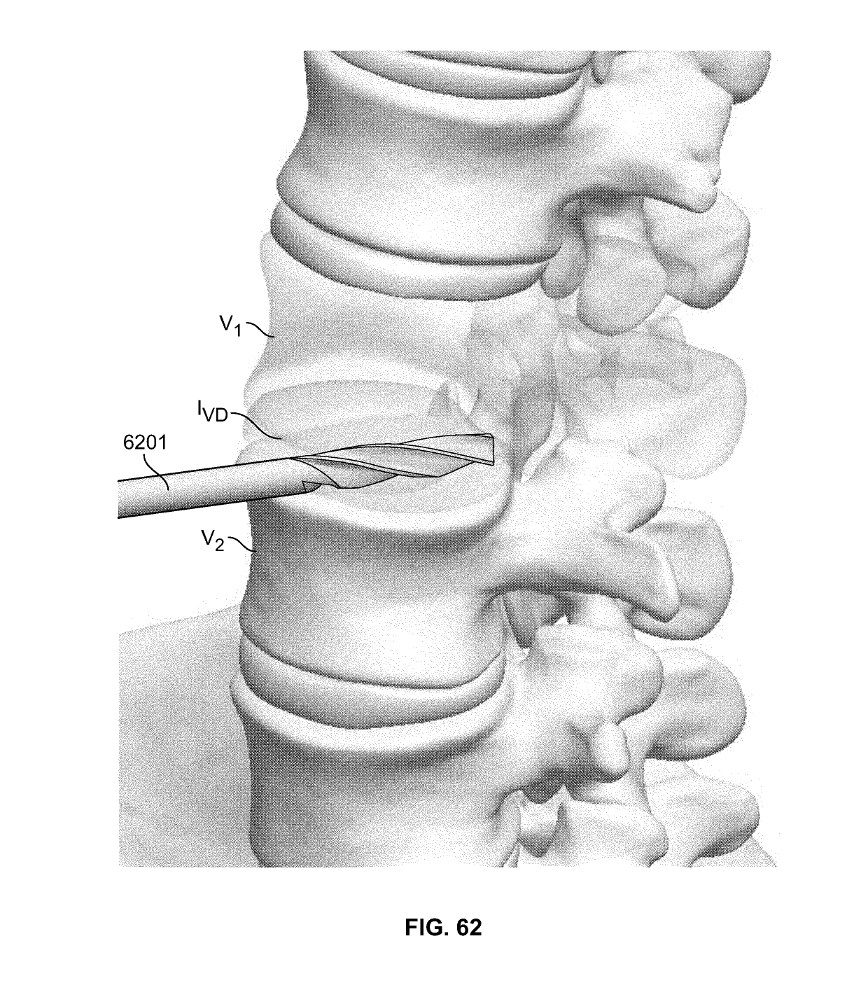

FIG. 62 shows illustrative drilling along guide wire 6101. Drilling may include advancing drill 6101 along guide wire 6101 to target site 6103. Drill 6101 may be advanced along guide wire 6101 to target site 6103. The drilling may continue to a depth in Ivd. The depth may be determined by using fluoroscopy or instrumentation.

Drilling may include manually dissecting tissue using kerrisons, rongeurs, curettes, or any other suitable tools.

Drilling may be performed along a first access path. The drill may then be removed from Ivd and positioned on Ivd such that a central axis of the drill is not coaxial with a central axis of the first access path. The drill may then be used to drill into Ivd along a second access path. Drilling along the second access path may augment a hole created by the drill along the first access path. Drilling along multiple access paths may create a chamber in Ivd that is elongated in the coronal plan. This may be desirable when a cage positioned in Ivd has a non-cylindrical and/or non-spherical expanded shape.

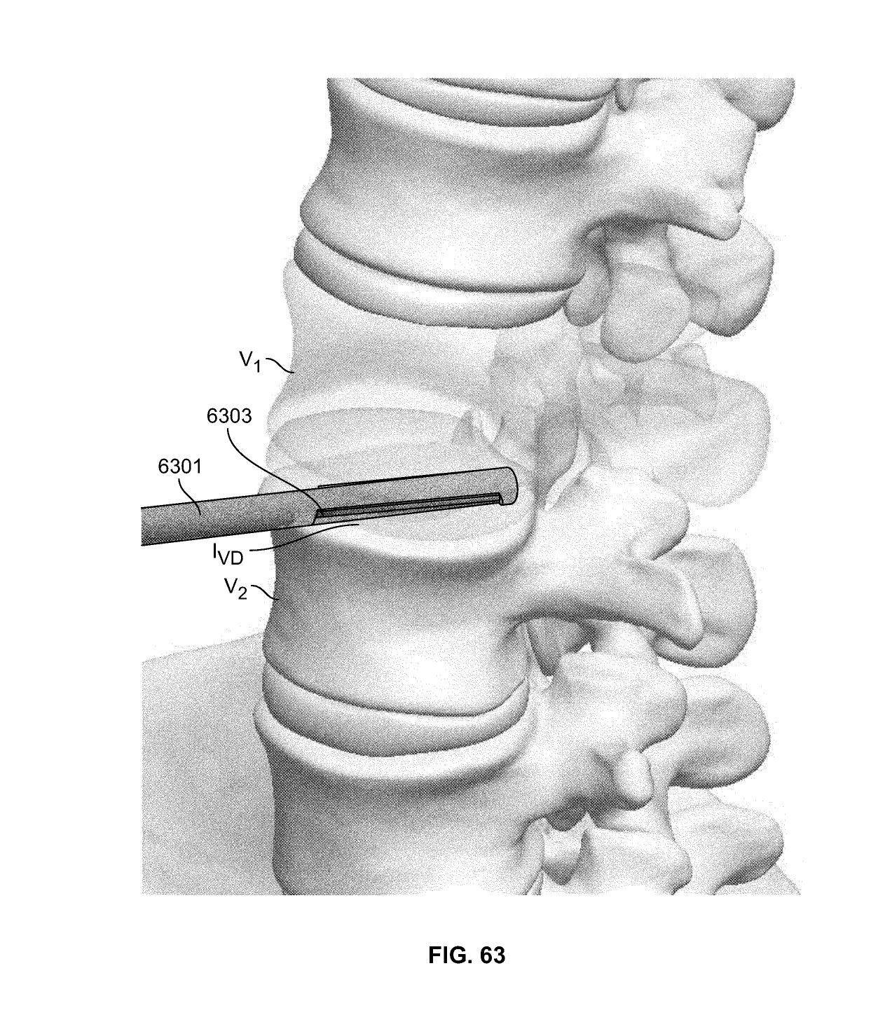

FIG. 63 shows illustrative placement of cavity preparation instrument 6301 in Ivd. Broaching member 6303 may be collapsed. Cavity preparation instrument 6301 may be the cavity preparation instrument. Broaching member 6303 may be the broaching member.

Broaching member 6303 may be activated. Cavity preparation instrument 6301 may form a first cavity within Ivd by being rotated about a central axis of cavity preparation instrument 6301. Rotating cavity preparation instrument 6301 may displace bone material in Ivd. Rotating cavity preparation instrument 6301 may distract and remove disc material in Ivd to form a cavity for a cage. Rotating cavity preparation instrument 6301 may also prepare a vertebral end plate of V1 and a vertebral end plate of V2 for implantation by scraping the end plates with the broaching member, thereby promoting bleeding bone. Bleeding bone may be desirable for its aid in promoting fusion.

When the drilling includes drilling along two or more access paths, the cavity preparation instrument may be inserted into Ivd along the first access path. The cavity preparation instrument may be rotated to form first cavity. The cavity preparation instrument may then be withdrawn from Ivd and advanced into Ivd along the second access path. The cavity preparation instrument may be rotated to form a second cavity.

The cavity preparation instrument may be inserted into Ivd after the drill has drilled along both the first and the second access path.

The cavity preparation instrument may be inserted into Ivd after the drill has formed the first access path but not the second access path. After forming a first cavity in Ivd, the cavity preparation instrument may be removed from Ivd. The drill may then drill along the second access path. The cavity preparation instrument may then be re-inserted into Ivd along the second access path and rotated to form a second cavity.

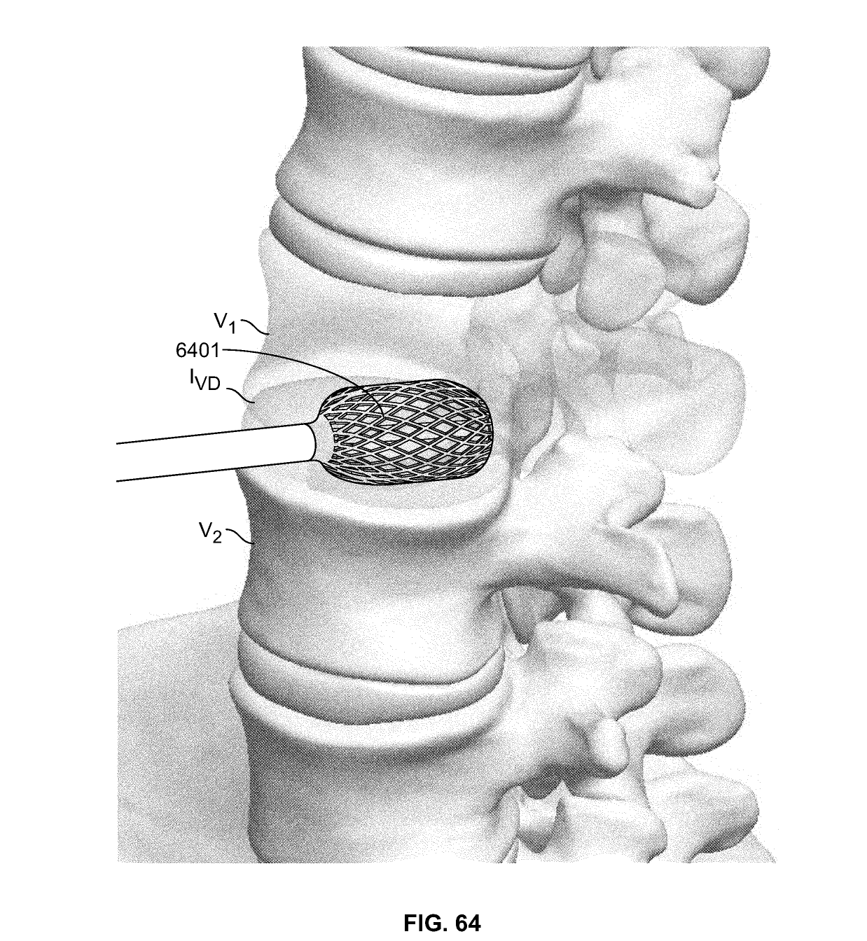

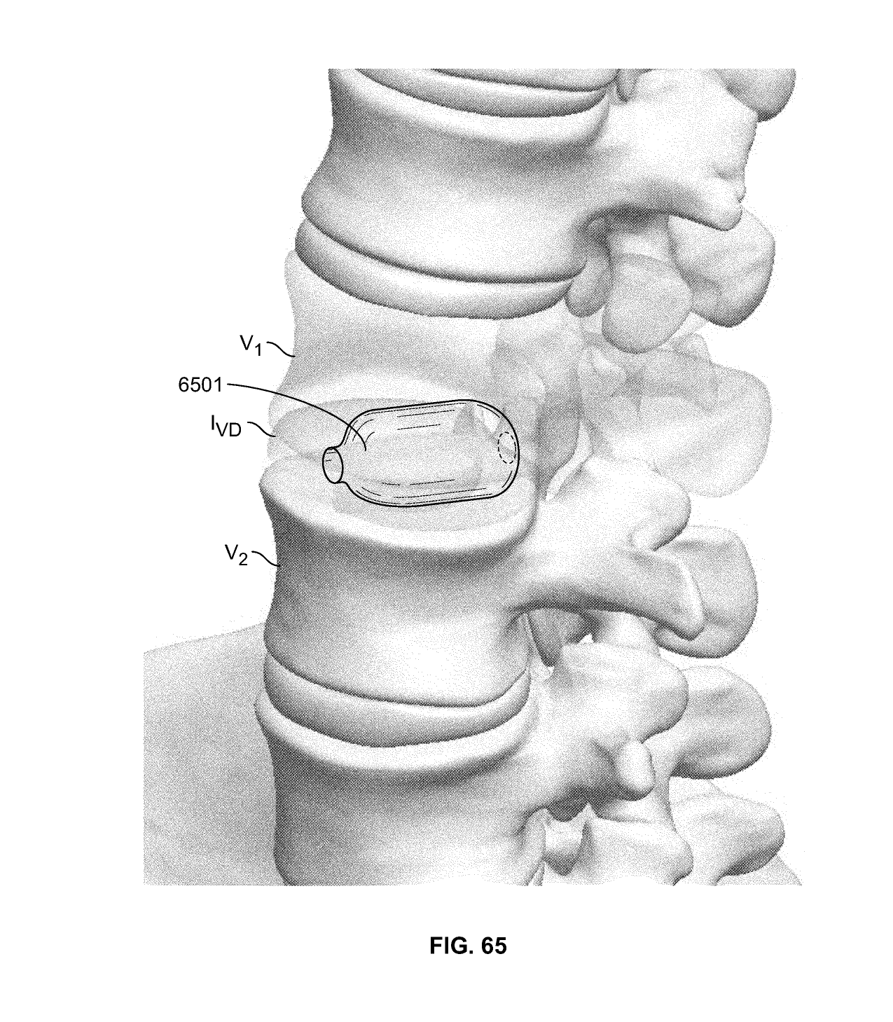

FIG. 64 shows illustrative step of augmenting a height of Ivd by expanding an instrument in Ivd (augmented height of Ivd not shown). In FIG. 64, the instrument is the cage. The instrument may be any suitable height restoring instrument known to those skilled in the art.

FIG. 65 shows illustrative cavity 6501 in Ivd. Cavity preparation instrument 6301 may form cavity 6501. Cavity 6501 may be formed, at least in part, by cage 6401, when cage 6401 is expanded in Ivd. Cavity preparation instrument 6301 may form some of cavity 6501. Cage 6401 may augment a height of the partially formed cavity when expanded.

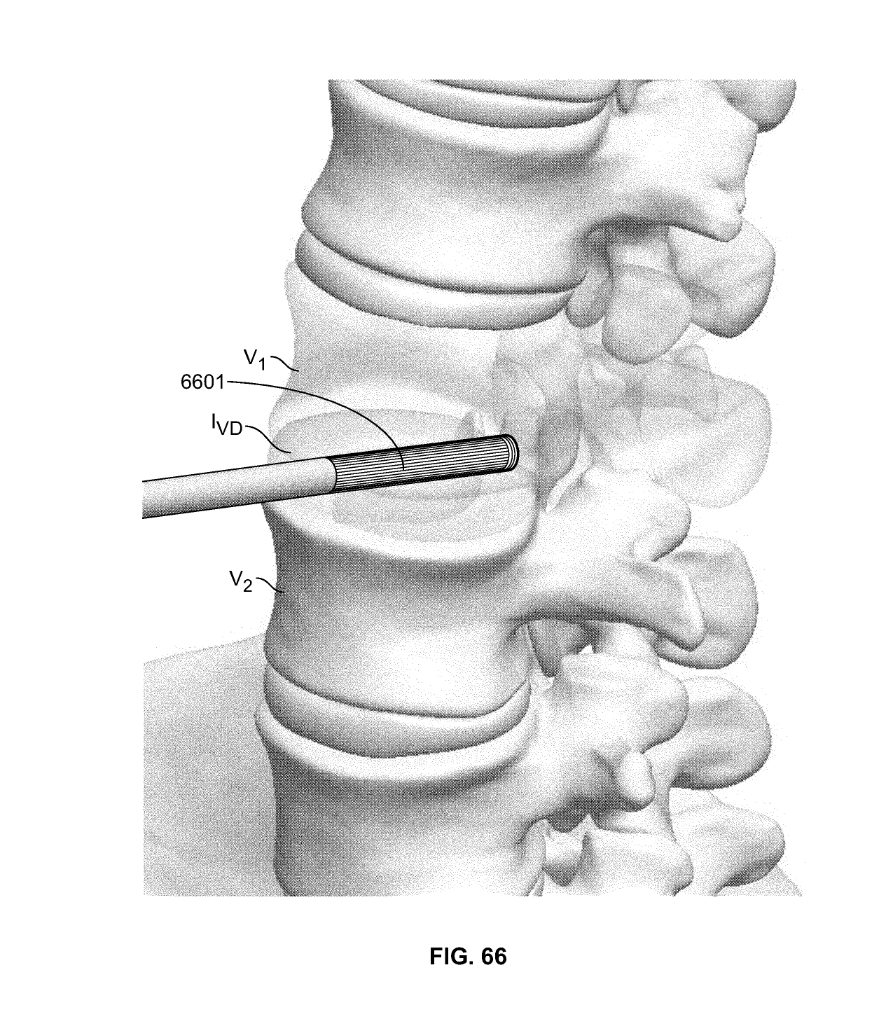

FIG. 66 shows illustrative positioning of cage 6601, in an unexpanded state, in Ivd. Cage 6601 may be positioned within cavity 6501. Cage 6601 may have one or more features in common with the first cage. Cage 6601 may have one or more features in common with the second cage.

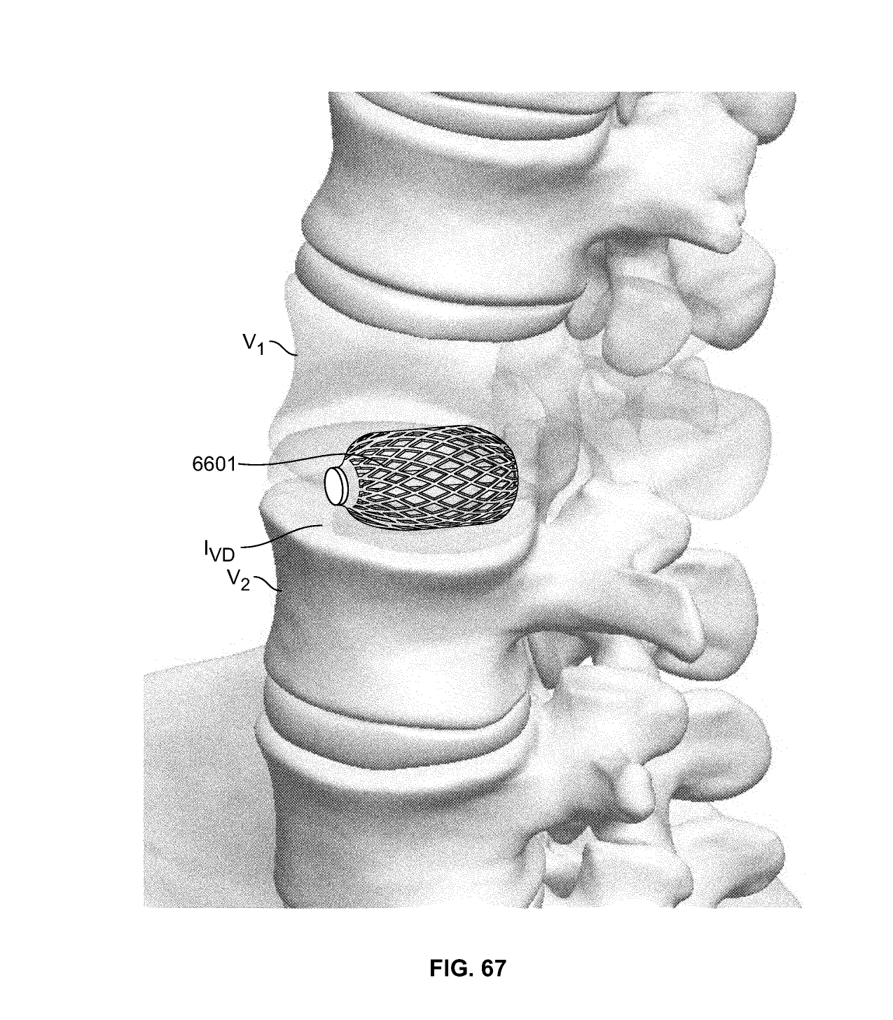

FIG. 67 shows illustrative cage 6601 in Ivd after deployment of cage 6601 in Ivd. Deploying a cage may include positioning the cage within a prepared cavity. Deploying the cage may include expanding the cage. Deploying the cage may include rotating the cage. The cage may be rotated by engaging a handle of the cage. Rotating the cage may further expand the cage. Rotating the cage may pack the cage with disc material. Rotating the cage may further prepare end plates to the contour of the cage. Optionally, material may be packed into the cage as desired. Material may be packed into the cage before or after deployment of the cage into the prepared cavity.

Cage 6601 is shown after expansion of cage 6601 and after removal of a handle previously coupled to cage 6601. Expanding cage 6601 may augment a height of Ivd (not shown).

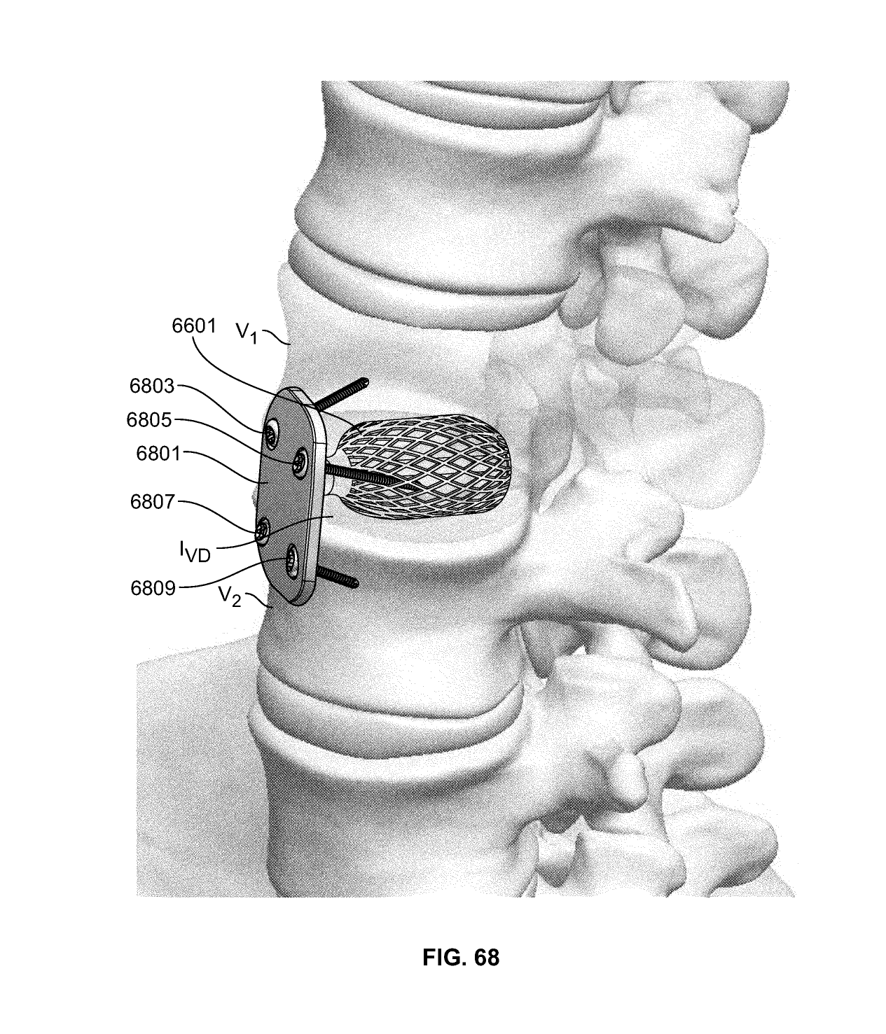

FIG. 68 shows illustrative supplemental fixation of V1 and V2 using plate 6601 and screws. Screws may engage either V1, V2, or one or two cells included in cage 6601. In FIG. 68, screw 6805 is shown engaging a cell of cage 6601. Screws 6803, 6807 and 6809 are shown engaging either V1 or V2. A screw may pass through V1, Ivd and V2.

A screw may engage a cell of cage 6601. A screw may engage two cells of cage 6601. A screw may engage two cells of 6601 and one or both of V1 or V2. A screw may pass through V1, Ivd and V2 without passing through cage 6601.

Driving one or more screws into cage 6601 may lock plate 6801 to cage 6601.

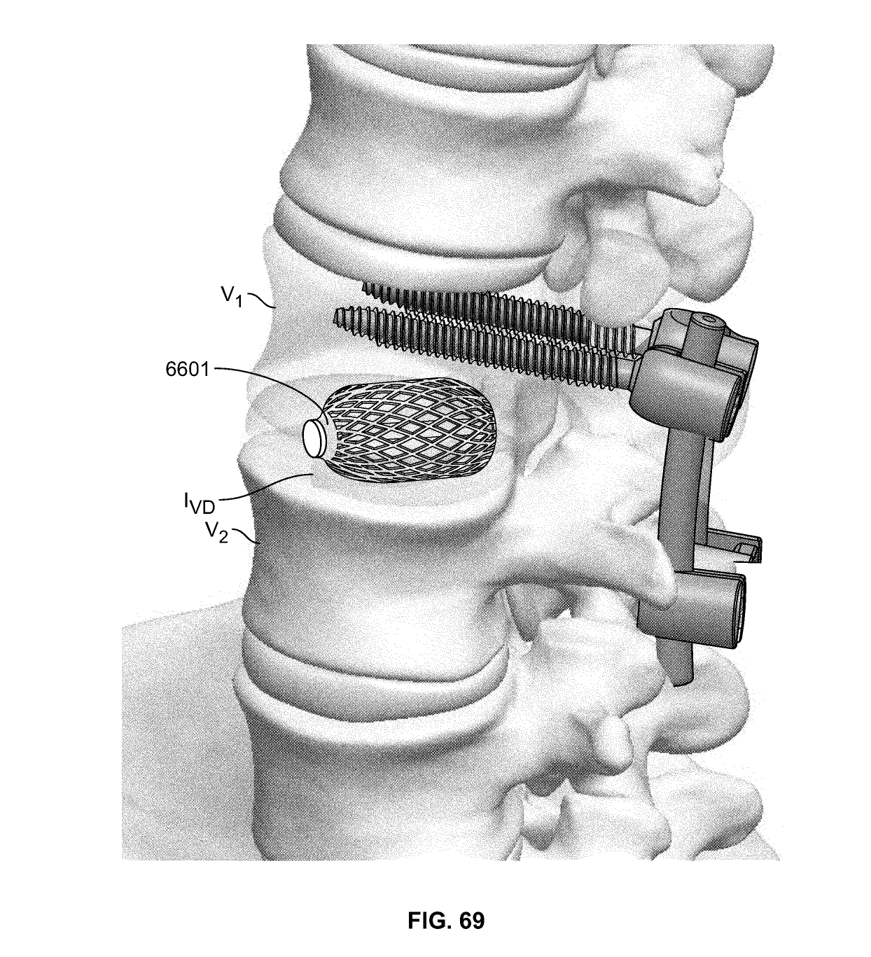

FIG. 69 shows illustrative supplemental fixation V1 and V2 using plate 6601 and pedicle screws. Screws may engage one of V1 or V2.

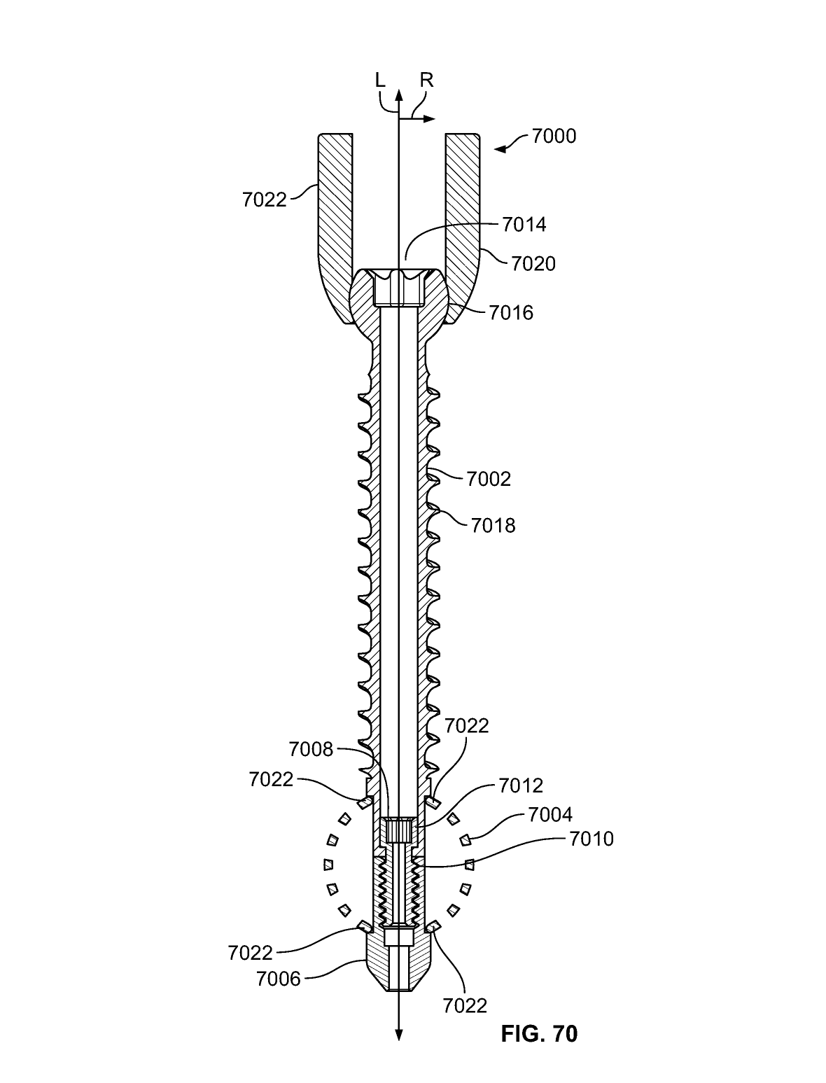

FIG. 70 shows a cross-section of illustrative pedicle screw 7000. Pedicle screw 7000 may include shaft 7002. Pedicle screw 7000 may include expandable mesh 7004. Expandable mesh 7004 is shown in an expanded state. Expandable mesh 7004 may be configured in a contracted state (not shown). Expandable mesh 7004 in a contracted state may be tubular. Pedicle screw 7000 may include tip 7006. Mesh 7004 may include T-shaped tabs 7022. Tabs 7022 may include transverse members forming the roof of the T. The transverse members run perpendicular to the cross-section. Receptacles on or about shaft 7002 may retain the transverse members and allow the tabs to change angle relative to axis L when the mesh expands or contracts. In the expanded state, the angle may be in the range of 30-35, 35-40, 40-45, 45-50, 50-55, 55-60, 60-65, 65-70, 70-75, 75-80, 80-85, 85-90 degrees.

Pedicle screw 7000 may be cannulated along longitudinal axis L. Expandable mesh 7004 may expand in radial direction R. The cannula may be used to advance pedicle screw 7000 along a guide instrument, such as a guidewire, a K-wire or any other suitable guide instrument.

Cannulated actuator screw 7008 may include distal thread 7010. Cannulated actuator screw 7008 may include proximal head 7012. Thread 7010 may engage internal threading (not shown) in the cannulated region of tip 7006. Shaft 7002 may longitudinally retain head 7012. A driver (not shown) may be introduced into proximal end 7014 of the cannula. The driver may be advanced along the cannula and seated in a receptacle (not shown) in head 7012. The driver may be used to turn head 7012. This may draw tip 7006 in the proximal direction and cause expansion of mesh 7004.

Head 7016 may receive a driver for rotating pedicle screw 7000 into tissue such as vertebra V. Vertebra V may be a vertebra shown in FIG. 2. Threads 7018 may cause pedicle screw 7000 to advance into vertebra V through an access hole prepared on a surface of the patient's body. Threads 7018 may engage the wall of the access hole and fix a position of tip 7006 in the interior of vertebra V.

Tulip head 7020 may be engaged in a swivel fashion with head 7016. Tulip head 7020 may be rotatable about axis L. Tulip head 7020 may be free to angulate with respect to axis L. Tulip head 7020 may include prongs 7022 in a U-shaped configuration to provide for fixation to an exterior fixation rod (not shown).

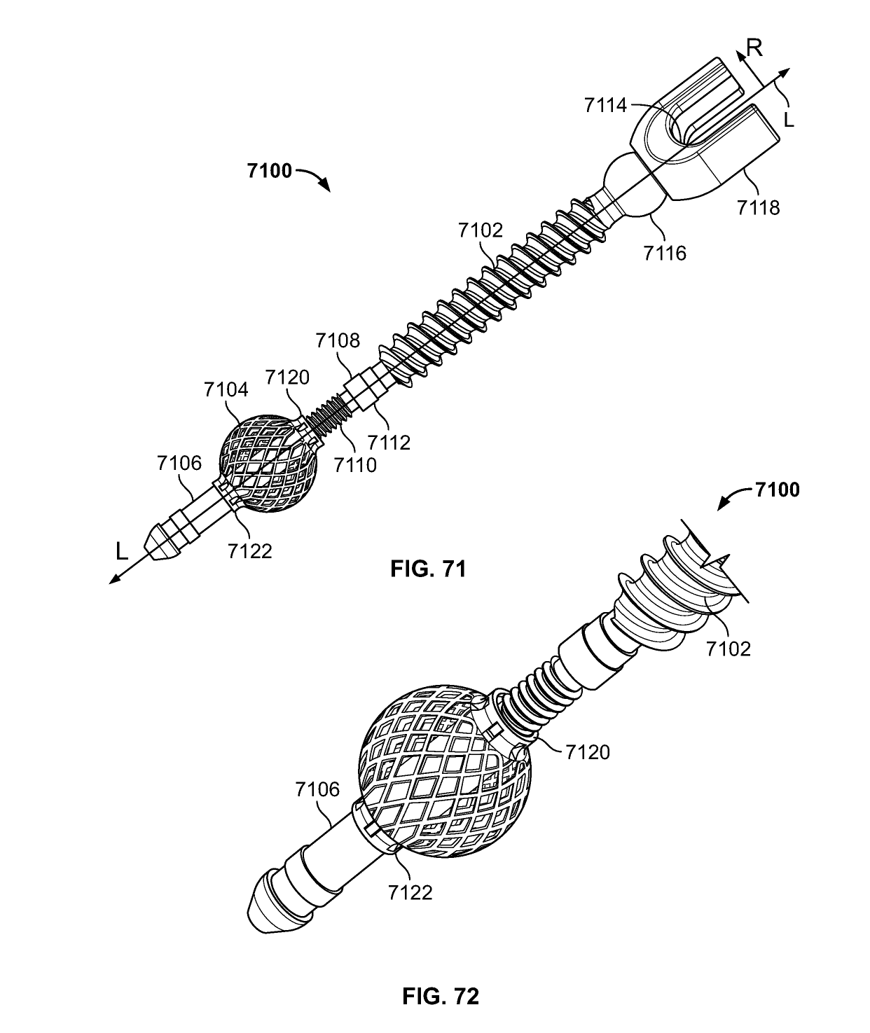

FIG. 71 shows a part cross-section of illustrative pedicle screw 7100. Pedicle screw 7100 may have one or more features in common with pedicle screw 7000 (shown in cross section in FIG. 70). Pedicle screw 7100 may include shaft 7102. Pedicle screw 7100 may include expandable mesh 7104. Expandable mesh 7104 is shown in an expanded state. Expandable mesh 7104 may be configured in a contracted state (not shown). Expandable mesh 7104 in a contracted state may be tubular. Pedicle screw 7100 may include tip 7106. Mesh 7104 may be fastened to collars 7120 and 7122. Collars 7120 and 7122 may be fixed to shaft 7102 and tip 7106, respectively.

Expandable mesh 7104 may have one or more features in common with the first cage. Expandable mesh 7104 may have one or more features in common with the second cage.

Pedicle screw 7100 may be cannulated along longitudinal axis L. Expandable mesh 7104 may expand in radial direction R. The cannula may be used to advance pedicle screw 7100 along a guide instrument, such as a guidewire, a K-wire or any other suitable guide instrument.

Cannulated actuator screw 7108 may include distal thread 7110. Cannulated actuator screw 7108 may include proximal head 7112. Thread 7110 may engage internal threading (not shown) in the cannulated region of tip 7106. Shaft 7102 may longitudinally retain head 7112. A driver (not shown) may be introduced into proximal end 7114 of the cannula. The driver may be advanced along the cannula and seated in a receptacle (not shown) in head 7112. The driver may be used to turn head 7112. This may draw tip 7106 in the proximal direction and cause expansion of mesh 7104.

Head 7116 may receive a driver for rotating pedicle screw 7100 into tissue such as vertebra V. Vertebra V may be a vertebra shown in FIG. 2. Threads 7118 may cause pedicle screw 7100 to advance into vertebra V through the access hole. Threads 7118 may engage the wall of the access hole and fix a position of tip 7106 in the interior of vertebra V.

Tulip head 7120 may be engaged in a swivel fashion with head 7116. Tulip head 7120 may be rotatable about axis L. Tulip head 7120 may be free to angulate with respect to axis L. Tulip head 7120 may include prongs 7122 in a U-shaped configuration to provide for fixation to an exterior fixation rod (not shown).

FIG. 72 shows some of pedicle screw 8300 from a perspective that is different from that shown in FIG. 71.

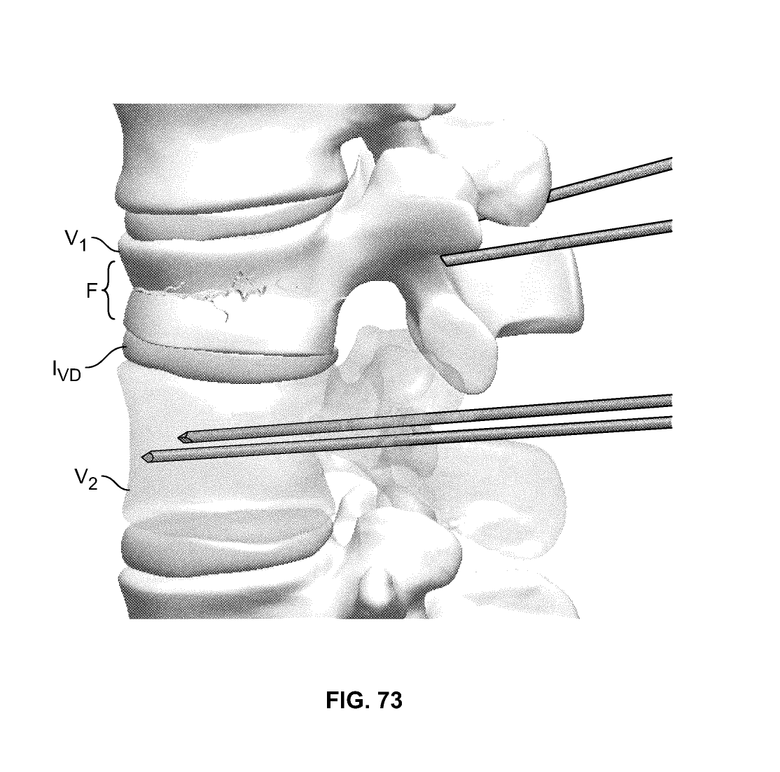

FIGS. 73-78 show an illustrative method for treating two spinal bodies. The spinal bodies may be vertebra V1 and vertebra V2. The method illustrated in FIGS. 73-78 may include additional method steps disclosed herein. The method may be performed in the order shown. The method may be performed in an order different from the order shown. The method may involve one or more of some or all of the apparatus shown in FIGS. 73-78.

The method illustrated in FIGS. 73-78 is shown using a bi-lateral, two-level approach. The method shown in FIGS. 73-78 may be performed using a unilateral approach. The method shown in FIGS. 73-78 may be performed at one or more levels, or elevations, along the spine.

The method illustrated in FIGS. 73-78 may be performed using an extrapedicular approach. The method illustrated in FIGS. 73-78 may be performed using a transpedicular approach. The method illustrated in FIGS. 73-78 may be performed using both an extrapedicular and a transpedicular approach.

FIG. 73 shows illustrative posterior placement of K-wires in spinal body V1, which has fracture F, and in spinal body V2.

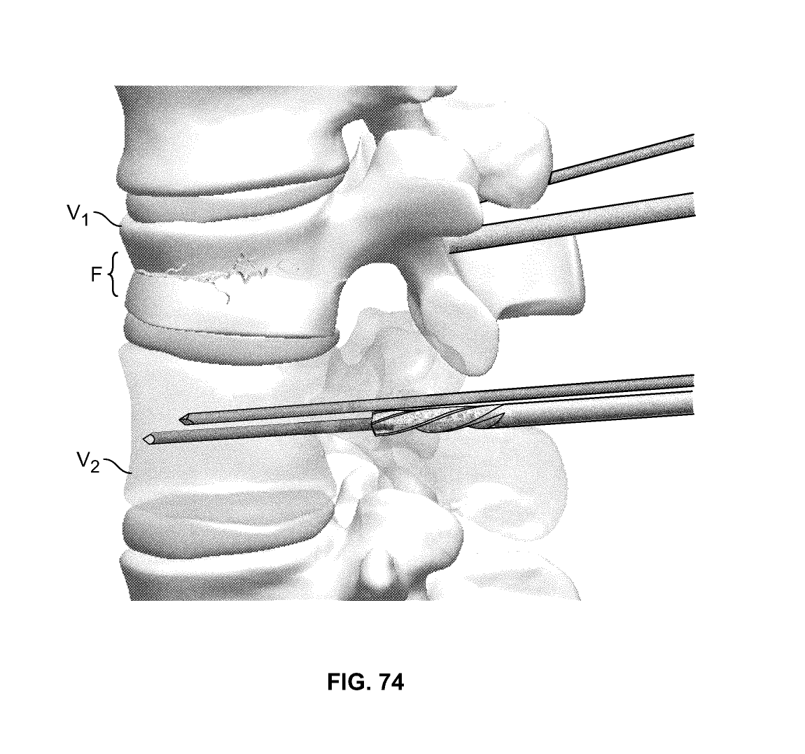

FIG. 74 shows illustrative access hole drilling over K-wires using a cannulated drill.

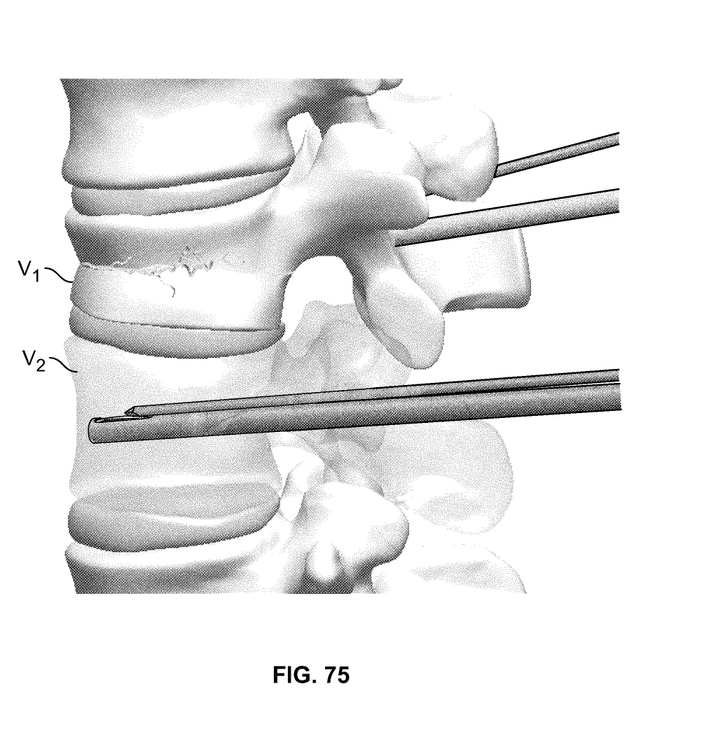

FIG. 75 shows illustrative insertion of the cavity preparation instruments in the access holes. The cavity preparation instruments have elongated rotators and expandable broaching members. The members are illustrated in the contracted state. The broaching members are expanded (not shown) and rotated to remove tissue from the vertebra to form cavities.

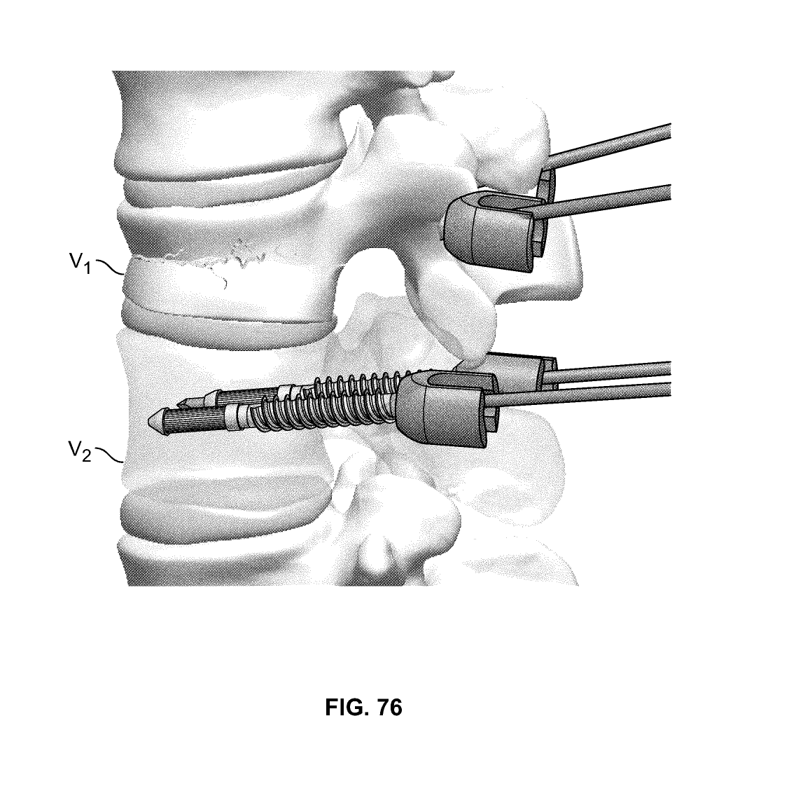

FIG. 76 shows illustrative placement of mesh-cage pedicle screws. The mesh-cage pedicle screws may have one or more features in common with one or more of pedicle screws 70 (shown in FIG. 70) and 71 (shown in FIG. 71). The pedicle screws are shown with mesh cages in the contracted state. In the contracted state, the mesh cages may be cylindrical. In the contracted state, the mesh cages may have concave-in (facing an axis such as L) curvature. The pedicle screws are shown as being cannulated and inserted over guide wires, such as K-wires. The pedicle screws may be pedicle screws that are not cannulated.

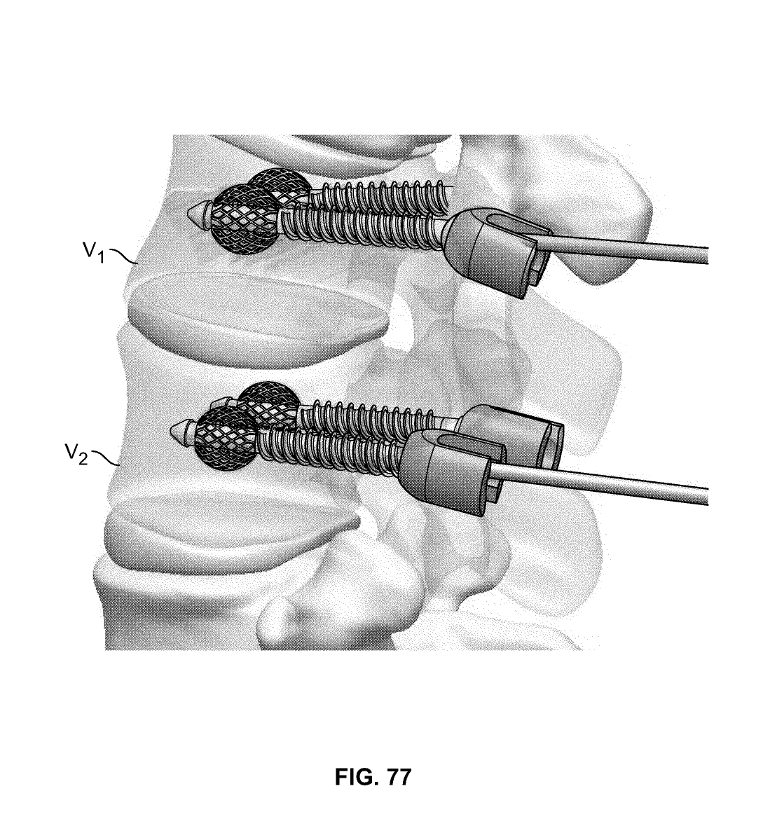

FIG. 77 shows illustrative expansion of the mesh cages inside the cavities.

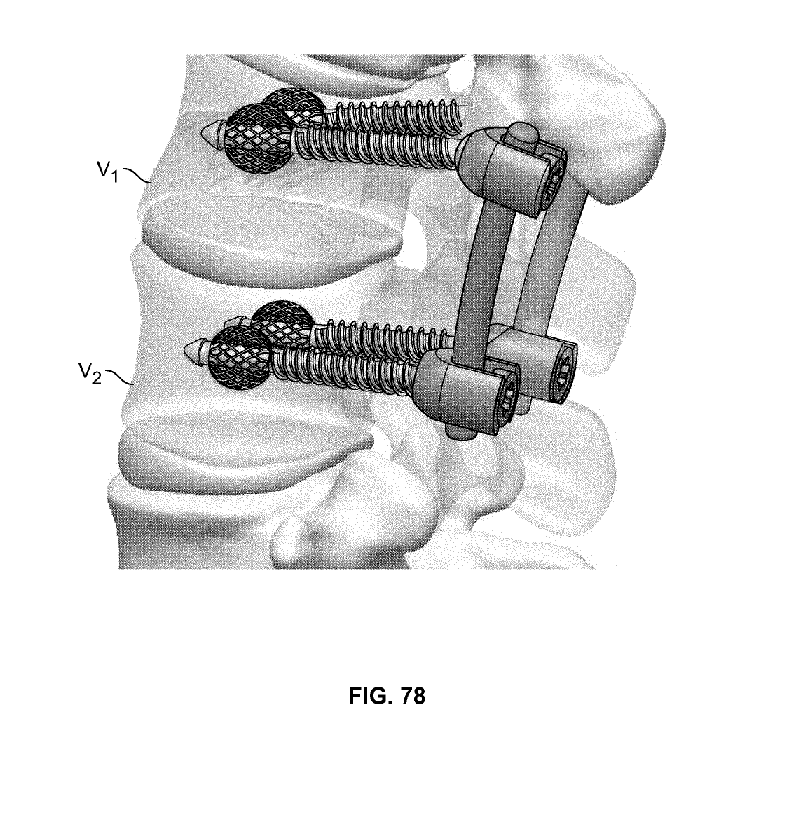

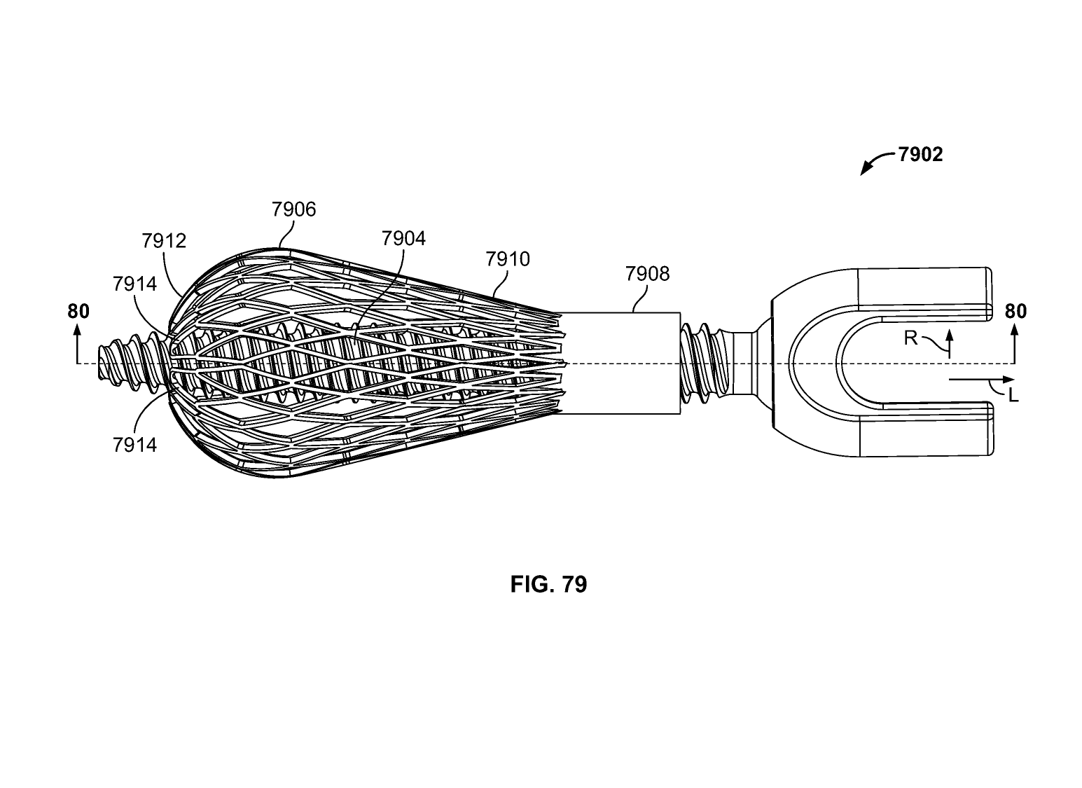

FIG. 78 shows illustrative fixation of the pedicle screws using exterior fixation rods.

FIG. 79 shows illustrative pedicle screw 7902. Pedicle screw 7902 may have one or more features in common with pedicle screw 7000.

Pedicle screw 7902 may include threaded member 7904. Pedicle screw 792 may include expandable cage 7906. Pedicle screw 7902 may include bushing 7908.

Proximal end 7910 of cage 7906 may be fixed to bushing 7908. Distal end 7912 of cage 7906 may include extensions 7914. Extensions 7914 may be biased toward longitudinal axis L. Extensions 7914 may be curved inward toward longitudinal axis L. Threaded member 7904 may deflect extensions 7914 away from axis L when threaded member 7904 advances past extensions 7914 in the distal direction. Threaded member 7904 may spread extensions 7914 apart from each other when threaded member 7904 advances past extensions 7914 in the distal direction. After expansion of cage 7906, extensions 7914 may terminate at or near longitudinal axis. Extensions 7914 may terminate in a zone defined by an inner radius (not shown) away from longitudinal axis L and an outer radius (not shown) away from longitudinal axis L. The outer diameter of the zone may be lesser than a major diameter of the threaded member. As such, when threaded member 7904 passes longitudinally by the zone, it deflects the extensions. Threaded member 7904 may radially deflect some or all of cage 7906.

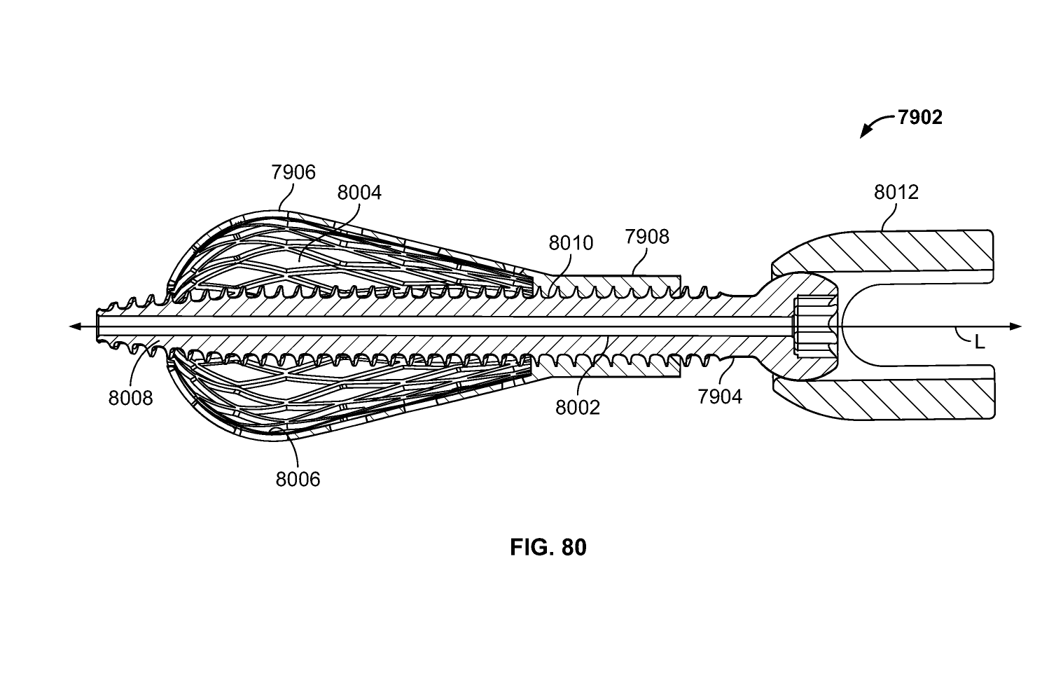

FIG. 80 shows cannula 8002 of threaded member 7904. Cannula 8002 may be used to flow a material through threaded member 7904 and back, through cage 7906, into space 8004 between threaded member 7904 and inner surface 8006 of cage 7906. One or more other cannulas (not shown) may be present in root 8008 of threaded member 7904, at an angle to cannula 8002, for flow of the material from cannula 8002 into space 8004. The other cannula may be in fluid communication with cannula 8002 and space 8004. The material may include cement, bone graft, biologically active material or any other suitable material.

Bushing 7908 may include internal threads 8010 for engagement with threaded member 7904.

Threaded member 7904 may be engaged with tulip head 8012 for fixation to other hardware.

Cage 7906 may be allowed to self-expand in a prepared cavity in a spinal body such as a vertebra. Threaded member 7904 may then be threaded through bushing 7908 and advanced distally through extensions 7914 and into a region of the spinal body distal extensions 7914. The material may be provided to space 8004.

Cage 7906 may have one or more features in common with the first cage. Cage 7906 may have one or more features in common with the second cage.

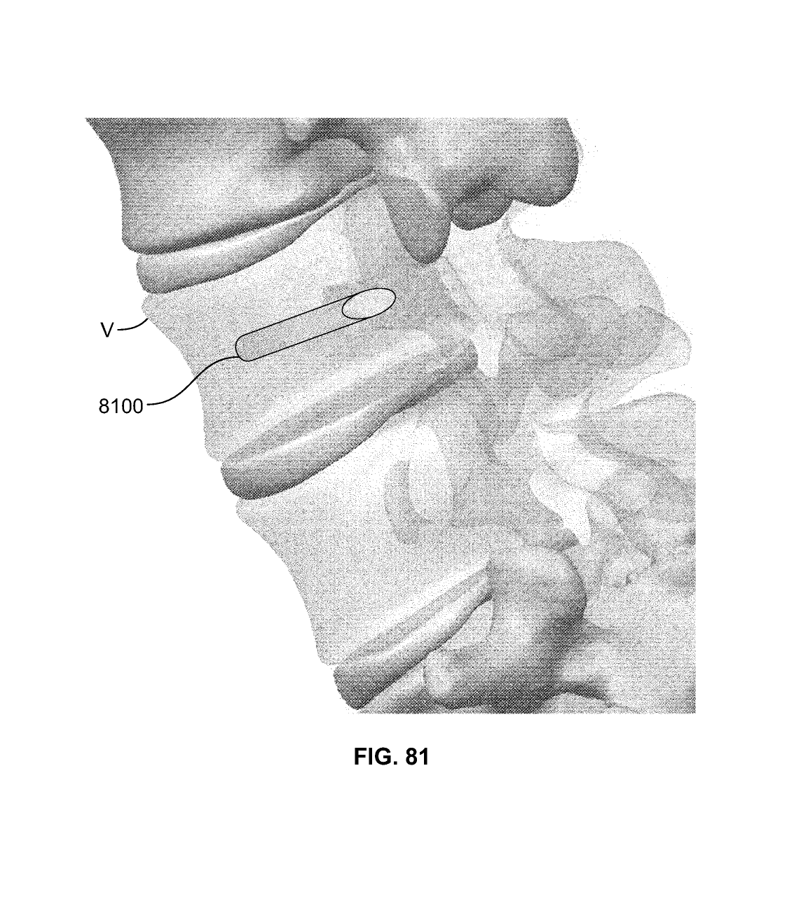

FIGS. 81-89 show an illustrative method for treating a spinal body. The spinal body may be intervertebral disc Ivd (“Ivd”). Ivd may be disposed between vertebra V1 and vertebra V2. The method illustrated in FIGS. 81-89 may include additional method steps disclosed herein. The method may be performed in the order shown. The method may be performed in an order different from the order shown. The method may involve one or more of some or all of the apparatus shown in FIGS. 81-89.

The method illustrated in FIGS. 81-89 is shown using a unilateral approach. The method shown in FIGS. 81-89 may be performed using a bilateral approach. The method shown in FIGS. 81-90 may be performed at multiple levels, or elevations, along the spine.

The method illustrated in FIGS. 81-89 may be performed using an extrapedicular approach. The method illustrated in FIGS. 81-89 may be performed using a transpedicular approach. The method illustrated in FIGS. 81-89 may be performed using both an extrapedicular and a transpedicular approach.

In FIGS. 81-89, vertebra V is accessed through a posterior side of V. V may be accessed through a posterior side during a Transforaminal Lumbar Interbody Fusion (“TLIF”) procedure or any other suitable procedure disclosed herein.

FIG. 81 shows the path of illustrative access hole 8100.

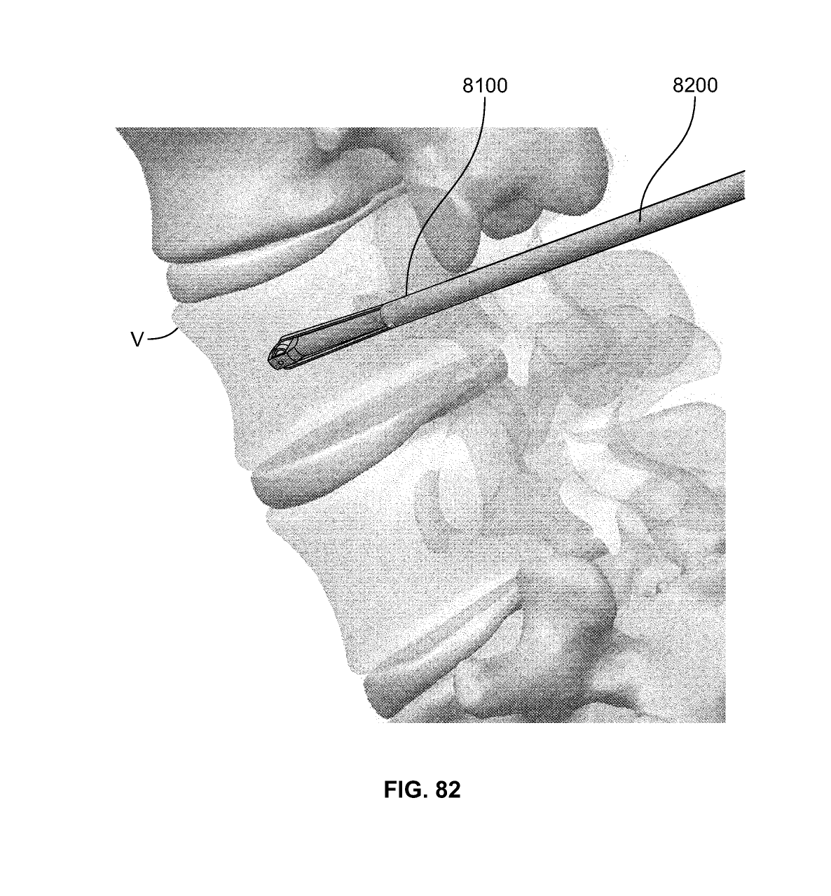

FIG. 82 shows illustrative placement of cavity preparation instrument 8200 in hole 8100. Cavity preparation instrument 8200 may be the cavity preparation instrument. Cavity preparation instrument 8200 is shown with a broaching member in a contracted state.

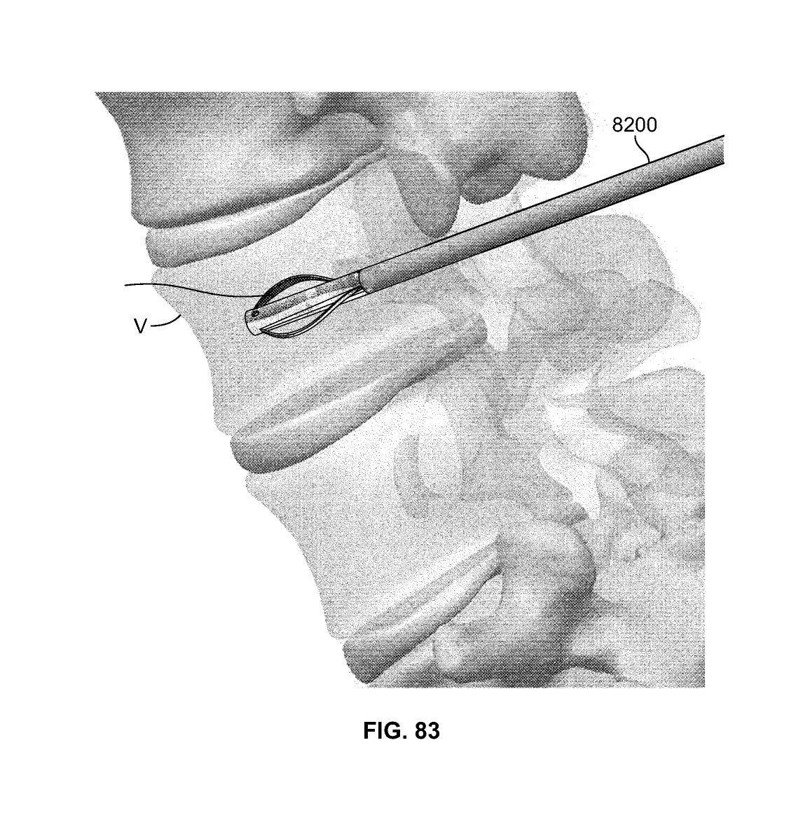

FIG. 83 shows cavity preparation instrument 8200 with the broaching member in an expanded state. Cavity preparation instrument 8200 may be rotated to prepare a cavity in which a mesh cage may be expanded.

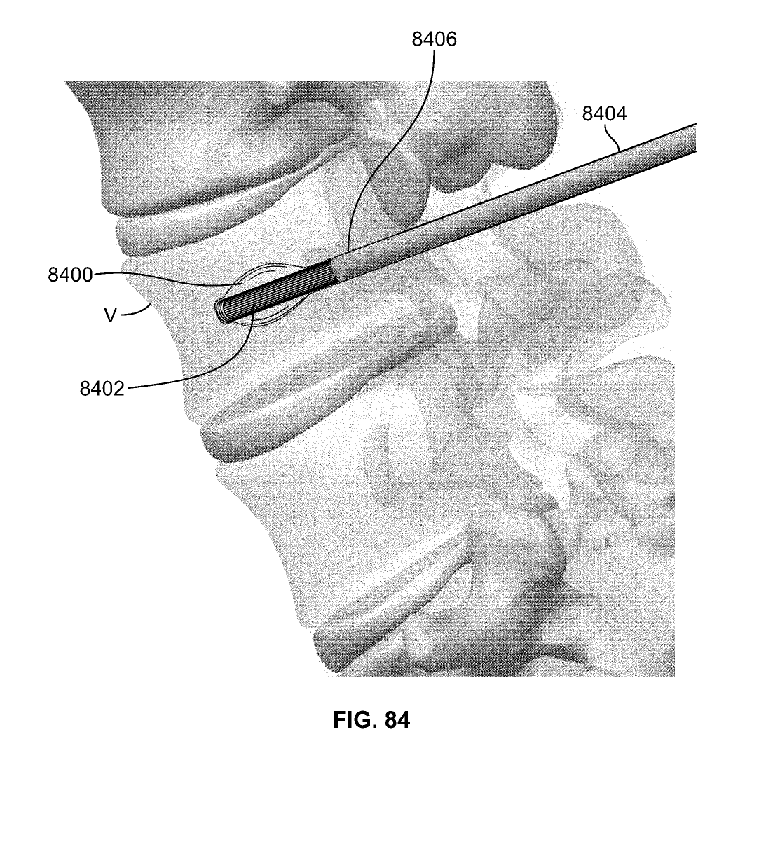

FIG. 84 shows cavity 8400 after removal of device 8200. Mesh cage 8402 (illustrated, schematically, in an unexpanded state) is placed inside cavity 8400 using handle 8404. Mesh cage 8402 may have one or more features in common with mesh cage 7906 (shown in FIG. 79). Mesh cage 8402 may be manually expanded. Mesh cage 7906 may be self-expanding. If mesh cage 7906 is self-expanding, mesh cage 7906 may expand gradually (not shown in schematic) as it is released from distal end 8406 of handle 8404.

Mesh cage 8402 may have one or more features in common with the first cage. Mesh cage 8402 may have one or more features in common with the second cage.

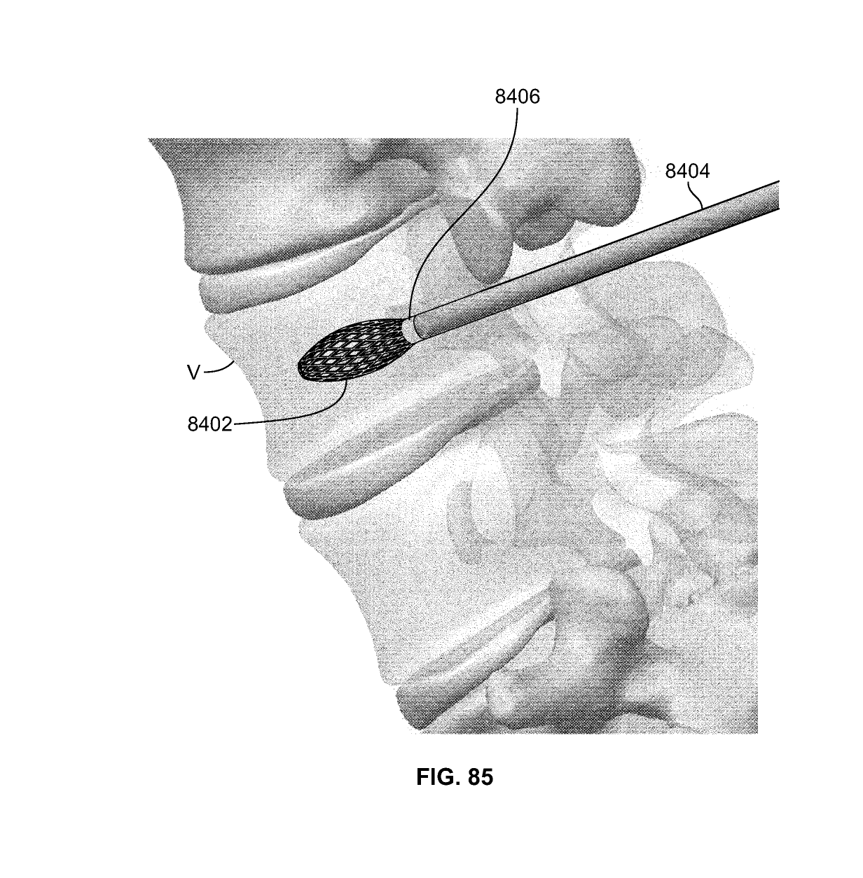

FIG. 85 shows cage 8402 in an expanded state in the cavity. Internally threaded bushing 8406 is present at the proximal end of cage 8402. Cage 8402 may include distal extensions (not shown) in a zone around a longitudinal axis of cage 8402.

Cage 8402 may be rotated in vertebra V using a handle of cage 8402. Rotating cage 8402 may expand cage 8402.

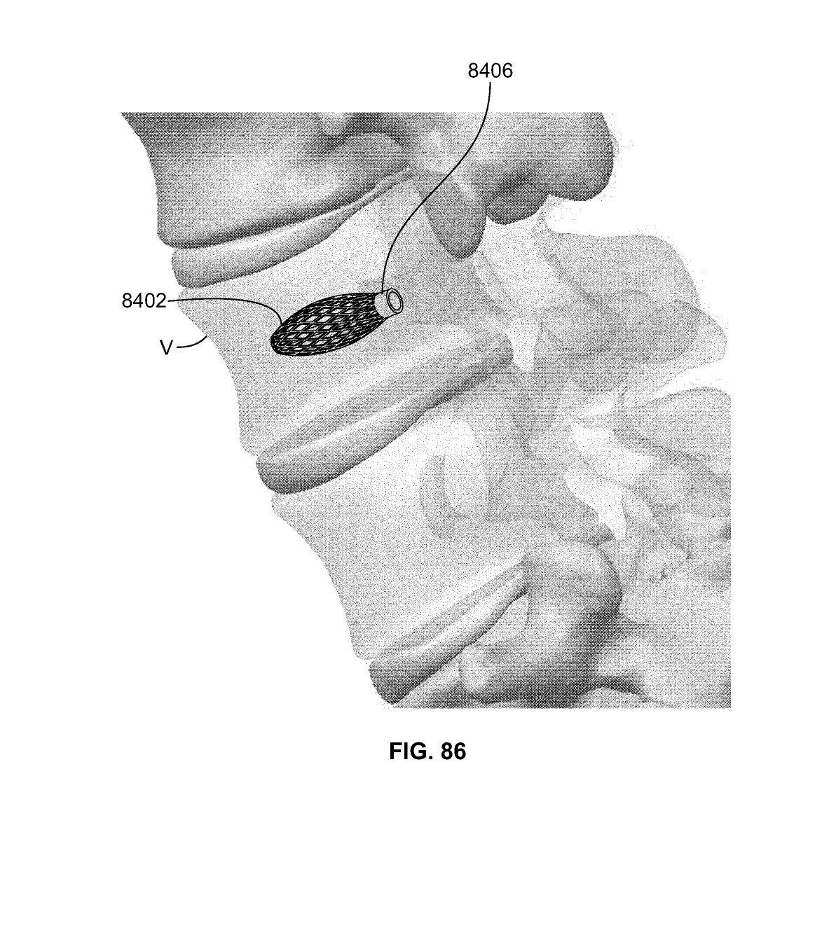

FIG. 86 shows cage 8402 and bushing 8406 (internal threads not shown) in the cavity after removal of handle 8404 (shown in FIG. 84).

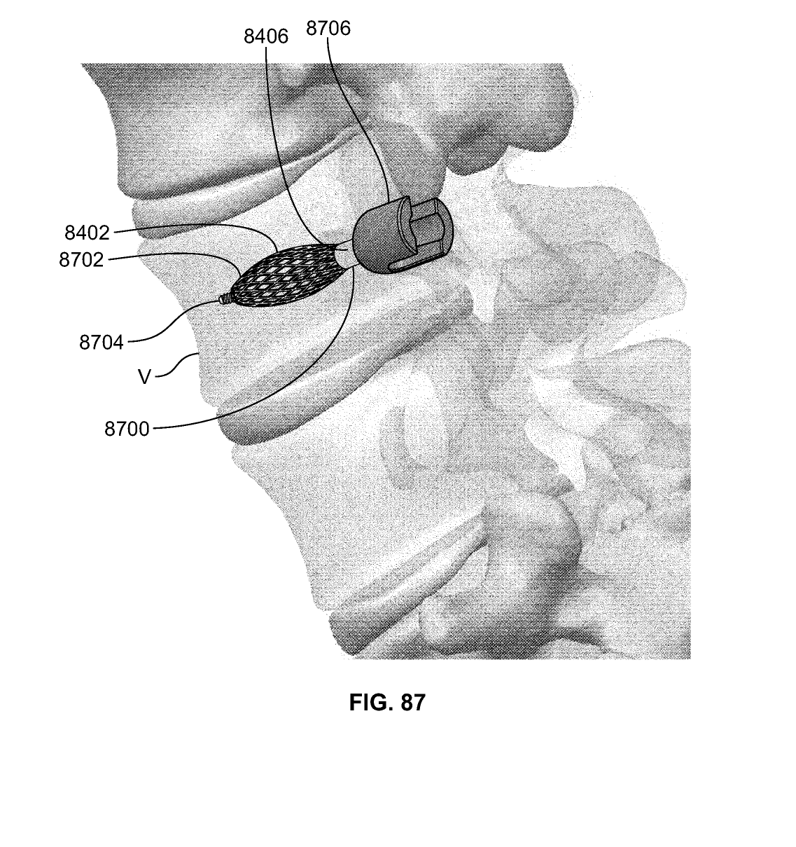

FIG. 87 shows screw 8700 threaded through bushing 8406 and extending past distal end 8702 of cage 8402. Tip 8704 of screw 8700 may extend into and may engage tissue of vertebra V. Tulip head 8706 is engaged with a head (not shown of screw 8700).

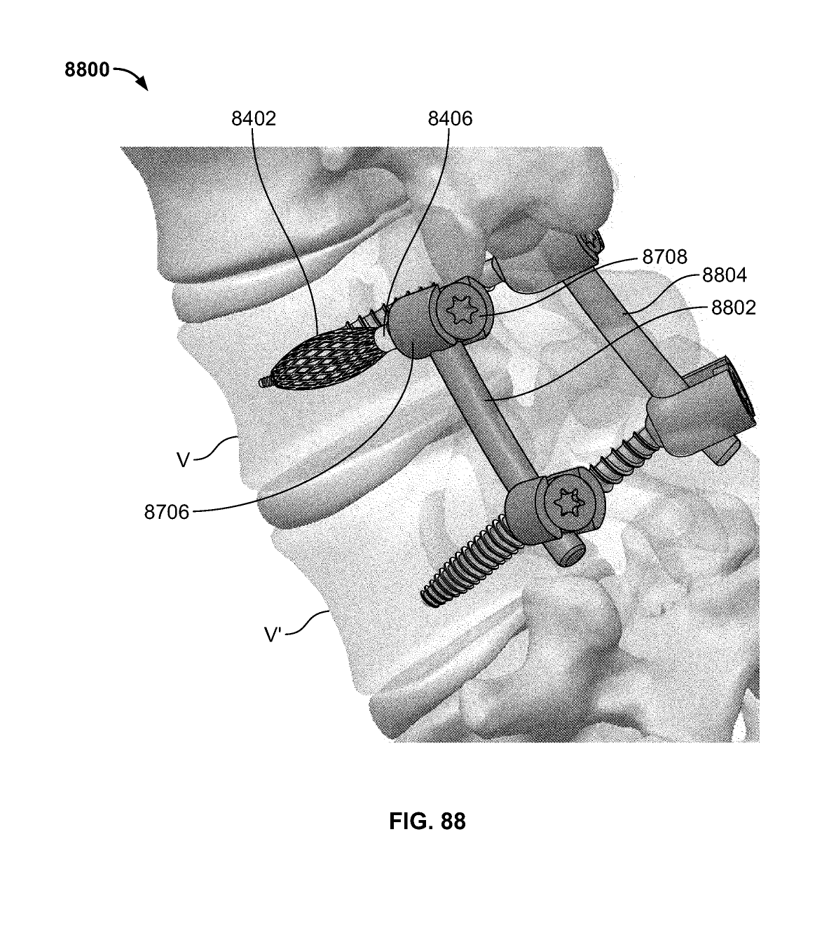

FIG. 88 shows illustrative bilateral posterior pedicle screw arrangement 8800 in connection with vertebrae V and V′. Pedicle screws are tied together with external fixation rods 8802 (left) and 8804 (right). One of the pedicle screws, on left, in vertebra V, is that shown in FIG. 87. Rod 8802 is fixed to tulip head 8706 by set screw 8708. One or more of the other pedicle screws in arrangement 8800 may have one or more features in common with the FIG. 87 pedicle screw. One or more of the other pedicle screws in arrangement 8800 may include one or more features that is different from those of the FIG. 87 pedicle screw. One or more of the pedicle screws in arrangement 8800 may have any suitable pedicle screw features.

Cage 8402 may be used together with other expandable cages disclosed herein in one or both of vertebra V and/or vertebra V′.

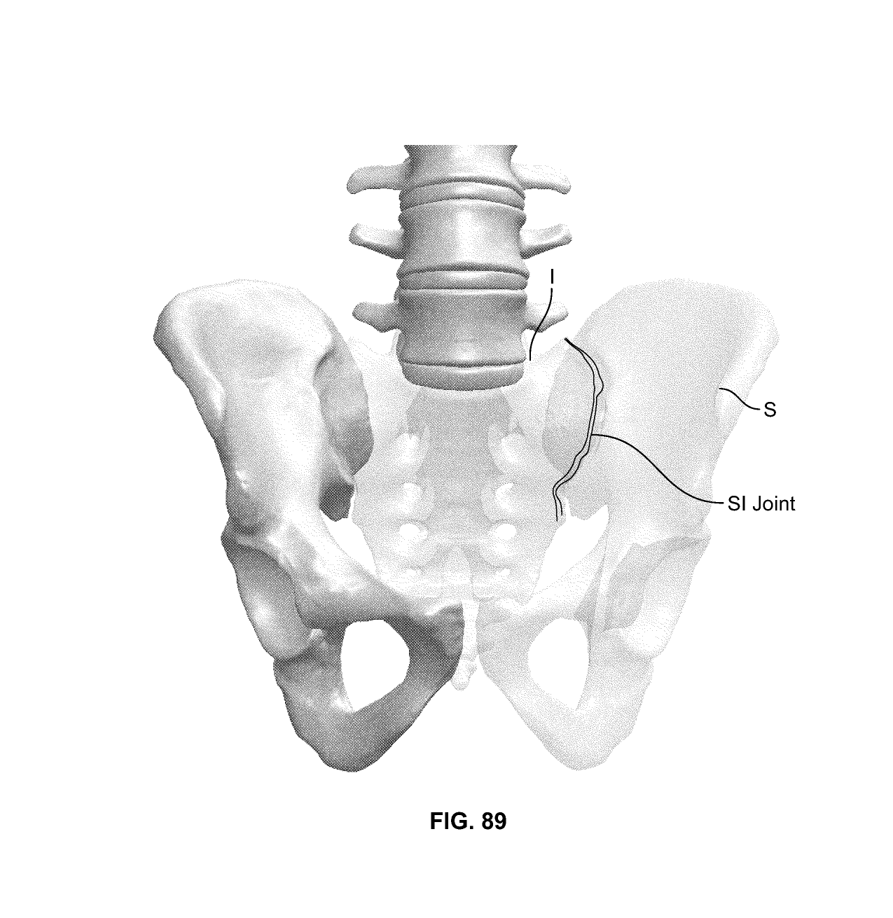

FIG. 89 shows an illustrative portion of a skeleton. The illustrated portion includes the sacrum bone S and the ilium bone I of a pelvis. FIG. 89 also illustrates Sacro-Iliac joint SI. The Sacro-Iliac Joint is a joint between sacrum bone S and ilium bone I.

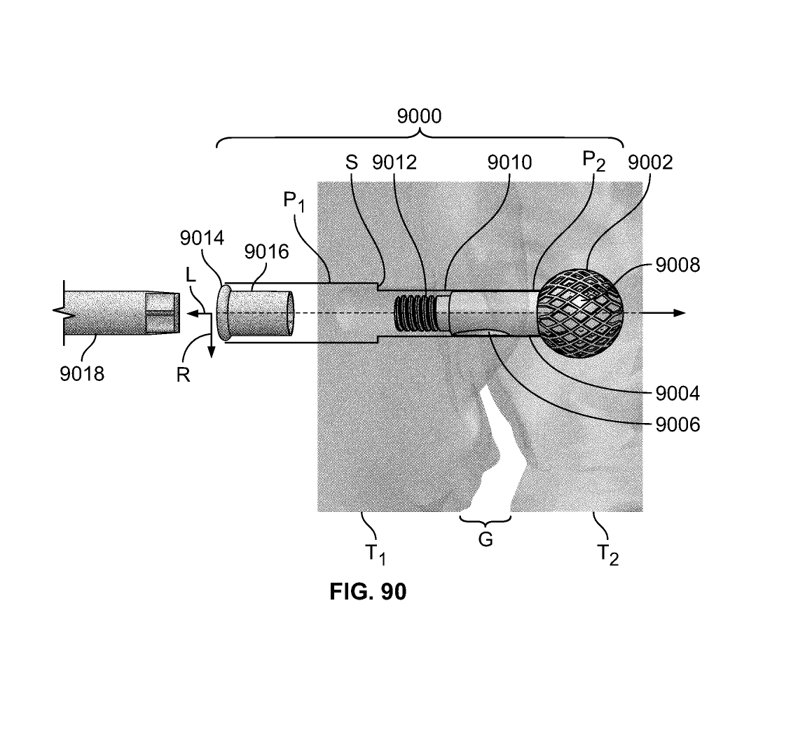

FIGS. 90-97 show an illustrative method for reducing a gap between a first body tissue and a second body tissue. The method illustrated in FIGS. 90-97 may include additional method steps disclosed herein. The method may be performed in the order shown. The method may be performed in an order different from the order shown. The method may involve one or more of some or all of the apparatus shown in FIGS. 90-97.

The method illustrated in FIGS. 90-97 may be performed unilaterally or bilaterally. The method illustrated in FIGS. 90-97 may be performed from two, three or more positions extending across the sacroiliac joint.

FIG. 90 shows illustrative apparatus 9000 for reducing gap G between bone tissue T1, which may be part of the ilium, and bone tissue T2, which may be part of the sacrum. T1 and T2 may together form a sacroiliac joint. The sacroiliac joint may separate by gap G.

Apparatus 9000 may include expandable cage 9002. Cage 9002 may have one or more features in common with the first cage. Cage 9002 may have one or more features in common with the second cage. Cage 9002 may be placed in a cavity prepared in bone tissue T2.

Cage 9002 may be hinged to base 9004. Base 9004 may be placed in access paths P2 in tissues T1 and T2, respectively. Threaded stem 9012 may extend from base 9004.

Base 9004 may define channel 9006, which may be oblique to longitudinal axis L. Channel 9006 may direct an anchor into cage interior 9008.

Plate 9014 may be affixed to tube 9016. Tube 9016 may have internal threads (not shown). Plate 9014 and tube 9016 may be inserted into path P1, which may have a diameter larger than that of path P2. Shoulder S may be present at the transition between P1 and P2. Tube 9016 may be threadingly engaged with the threads of threaded stem 9012. As tube 9016 is advanced longitudinally along stem 9012, plate 9014 may come into contact with shoulder S. Further threading, with plate 9014 in contact with shoulder S, may draw tension on base 9004. This may pull on cage 9002. Plate 9014 and cage 9002 may thus apply compression to tissues T1 and T2 to reduce or close gap G.

Driver 9018 may be used to apply torque plate 9014 and tube 9016.

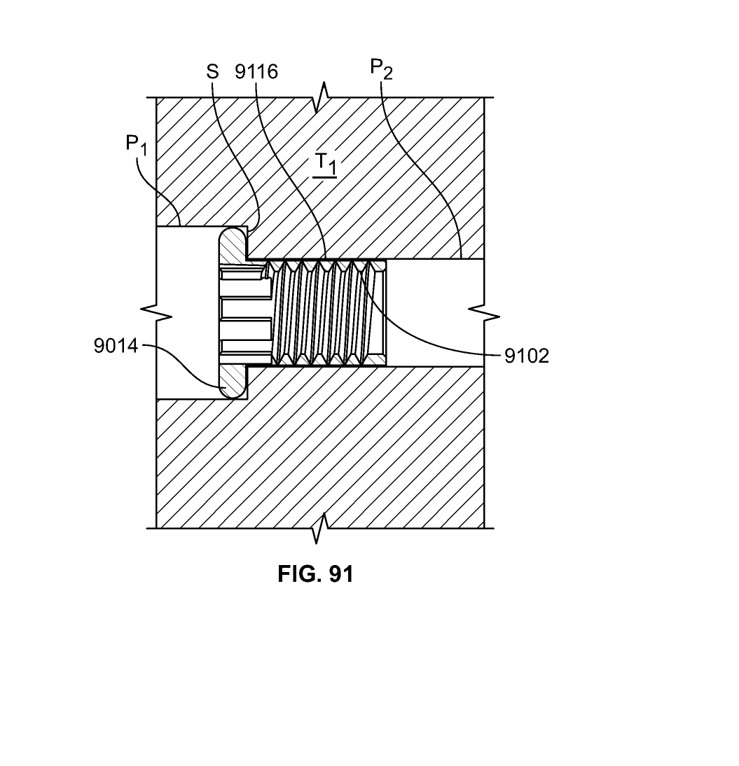

FIG. 91 shows tube 9016 in path P2 with plate 9014 seated against shoulder S. In operation, threaded stem 9012 (shown in FIG. 90) would be threadedly engaged with threads 9102, which would be drawn into tension between plate 9016 and cage 9002 (shown in FIG. 90).

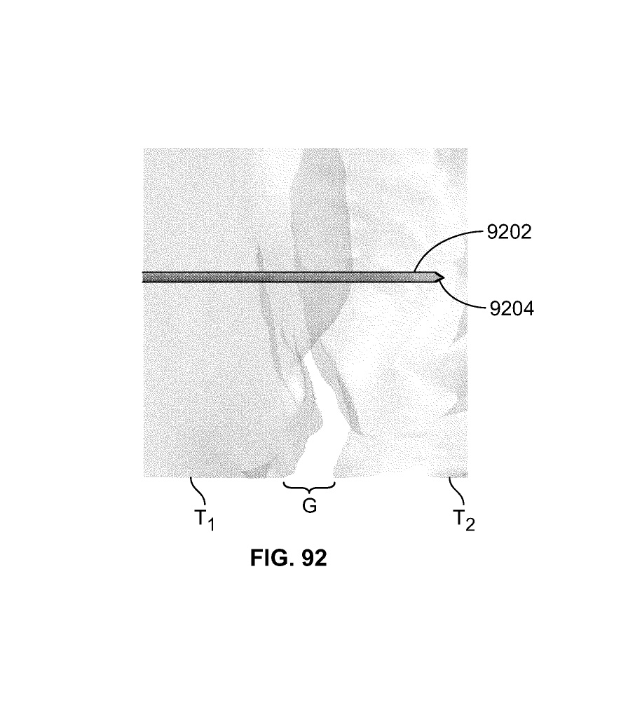

FIG. 92 shows guide wire 9202 piercing tissue T1 and T2 and traversing gap G. Tip 9204 of guide wire 9202 is placed where the distal end of the cage is desired to be disposed. One or both of anterior-posterior lateral placement of guide wire 9202 may be confirmed by medical imagery.

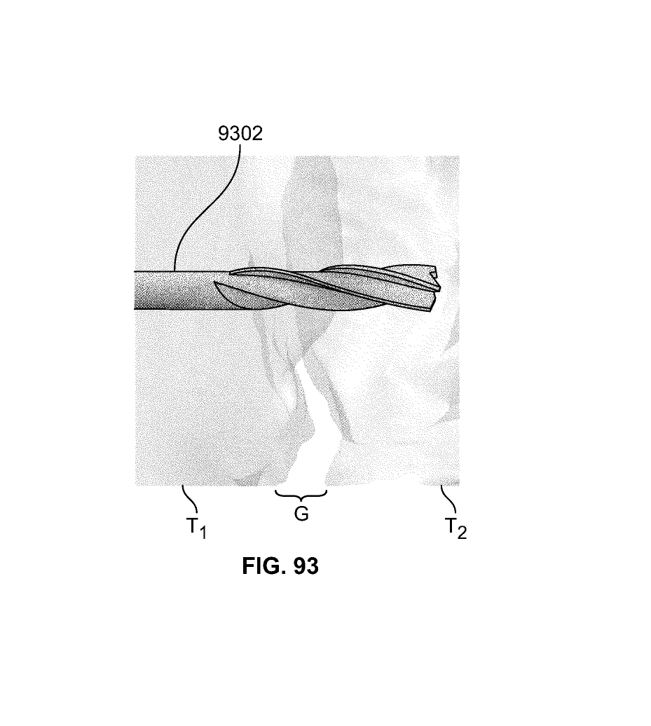

FIG. 93 shows drill 9302 advanced along the path established by guide wire 9202 (shown in FIG. 92). Drill 9302 may be cannulated, and may be advanced over guide wire 9202. Drill 9302 may thus clear path P2 (shown in FIG. 90). A diameter of drill 9302 may be selected. The diameter may be selected to provide a path P2 diameter that matches one or more of an outer diameter of a cavity preparation instrument, in a contracted state, and a mesh cage, in a contracted state.

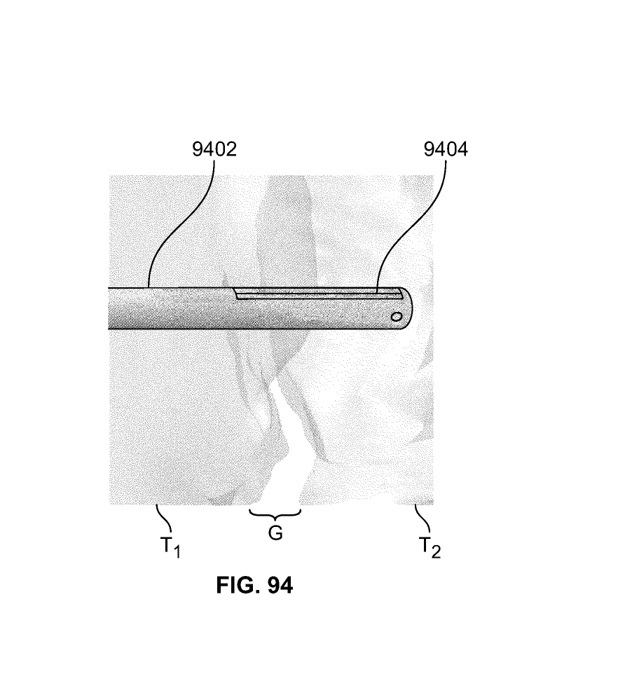

FIG. 94 shows cavity preparation instrument 9402 in path P2. Device 9402 may be the cavity preparation instrument. Device 9402 is shown in a contracted state. Device 9402 may include expandable broaching member 9404.

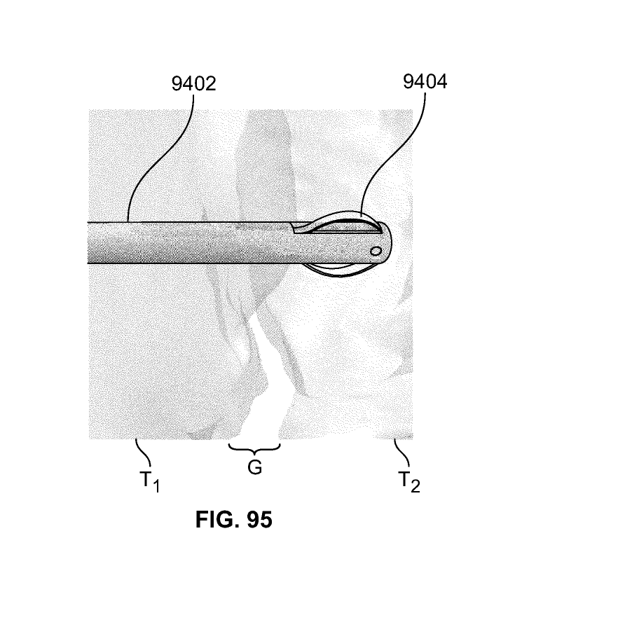

FIG. 95 shows broaching member 9404 partially expanded. Cavity preparation instrument 9402 may be the cavity preparation instrument. Broaching member 9404 may be the broaching member.

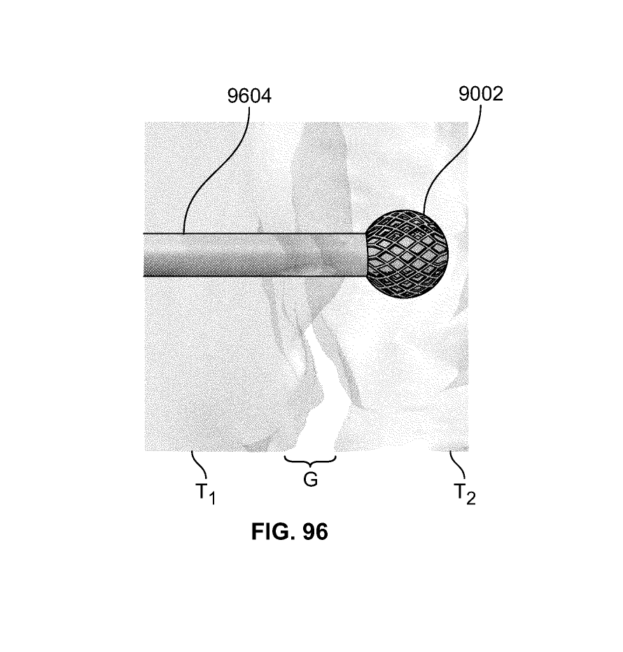

FIG. 96 shows cage 9002 expanded with a cavity in tissue T2 that was prepared by rotating cavity preparation instrument 9402. Tube 9604 may be a hollow delivery tube for placement of cage 9002. Cage 9002 may be self-expanding upon being urged out of the end of tube 9604.

Tube 9604 may be a tail that engages with threads on stem 9012. The tail may be used to position the mesh cage in the prepared cavity. The tail may then be removed so that plate 9014 may be deployed in path P1 and engaged with stem 9012.

Tube 9604 may be a base such as base 9004 (shown in FIG. 90). Tube 9604 may include an actuator such as a mechanism shown in FIG. 70. The actuator may be used to manually or robotically expand cage 9002. The actuator may be a driver that may be inserted into a cannula in tube 9604.

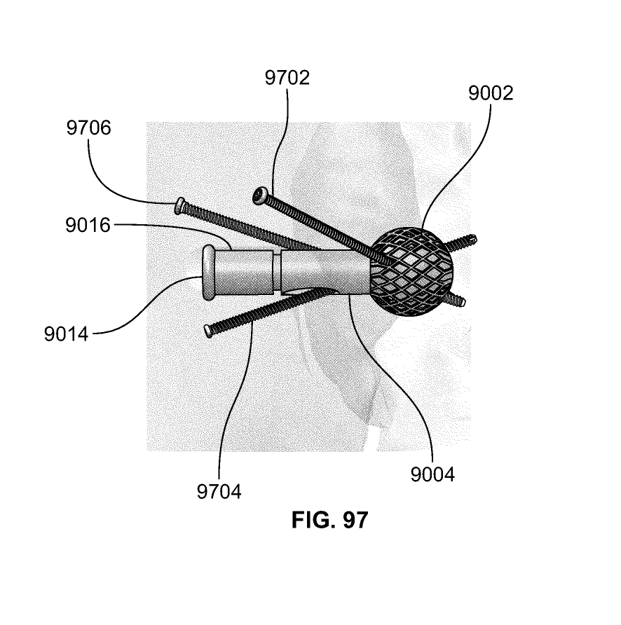

FIG. 97 shows device 9000 with anchors 9702, 9704 and 9706. Anchor 9702 penetrates bone tissue T2, enters the interior of cage 9002, exits cage 9002, and again penetrates bone tissue T2. Anchors 9704 and 9706 pass through clearance holes (such as 9006 (shown in FIG. 90)), cross the interior of cage 9002, and penetrate bone tissue T2. One or both of anchors 9704 and 9706 may engage bone tissue T1 before passing through a respective clearance hole. One or both of anchors 9704 and 9706 may engage bone tissue T2 before passing through a respective clearance hole.

A guide wire may be used to make a pilot trajectory for an anchor. Medical imagery may be used to observe creation of the pilot trajectory. Medical imagery may be used to help select an appropriate length, based on the pilot trajectory or other suitable factors, for an anchor.

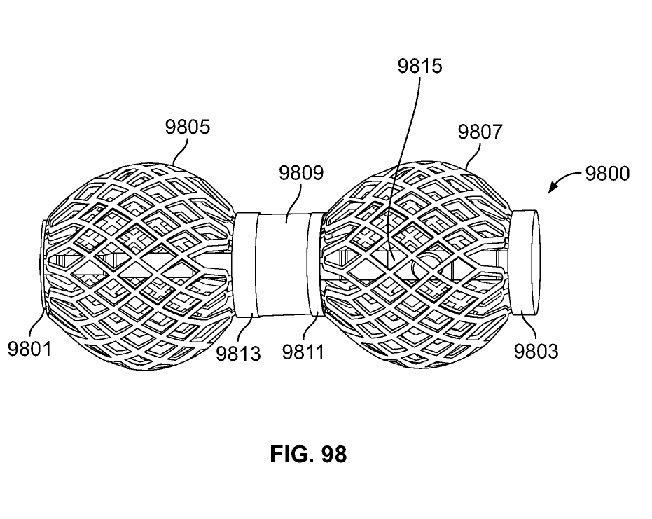

FIG. 98 shows illustrative apparatus 9800 for reducing gap G between bone tissue T1 (“B1,” in following figures), which may be part of the ilium, and bone tissue T2 (“B2,” in following figures), which may be part of the sacrum. T1 and T2 may together form a sacroiliac joint. The sacroiliac joint may separate by gap G.

Apparatus 9800 may include expandable cage 9805. Cage 9805 may have one or more features in common with the first cage. Cage 9805 may have one or more features in common with the second cage. Cage 9805 may be placed in a cavity prepared in bone tissue T2. Cage 9805 may be hinged to base 9813. Cage 9805 may be hinged to distal hub 9001. Cage 9805 may be self-expanding. If cage 9805 is self-expanding, it may be delivered through a sheath (not shown) and allowed to expand as it exits the sheath. Cage 9805 may be expanded by an actuator. The actuator may be housed in actuator assembly 9809.

Apparatus 9800 may include expandable cage 9807. Cage 9807 may have one or more features in common with the first cage. Cage 9807 may have one or more features in common with the second cage. Cage 9807 may be placed in a cavity prepared in bone tissue T2. Cage 9805 may be hinged to base 9811. Cage 9805 may be hinged to proximal hub 9803. Cage 9807 may be self-expanding. If cage 9807 is self-expanding, it may be delivered through a sheath (not shown) and allowed to expand as it exits the sheath. Cage 9807 may be expanded by an actuator. The actuator may be housed in actuator assembly 9809.

The actuators may be cannulated to permit access by a driver for drawing the cages toward each other.

Central axis member 9815 may include one or both of the actuators.

Central axis member 9815 may run from proximal hub 9803 to distal hub 9801. Central axis member 9815 may run through actuator assembly 9809. Central axis member 9815 may include a first member that runs from proximal hub 9803 to assembly 9809, and a second part that runs from assembly 9809 to distal hub 9001. Central axis member may include an adjustable-length component. The adjustable-length component may be housed in actuator assembly 9809. Central axis member 9815 may be cannulated. The cannula may provide access by a driver, to actuator assembly 9809, when cages 9805 and 9807 are disposed in tissue T1 and T2, respectively.

Actuator assembly 9809 may have an adjustable-length component. The adjustable length components may include mutually threaded members that change length when rotated relative to each other.

The adjustable length components may include; a central twin-threaded member having one end threaded clockwise and another end threaded counterclockwise. Each threading may be mutually threaded with a correspondingly threaded member. Rotation of the twin-threaded member relative to the correspondingly threaded members may lengthen or shorten the component.

The driver may be keyed to induce relative rotation between threaded members. For example, the driver may have a static shaft that holds one member, and a rotatable sleeve that includes a lateral extension that engages and rotates another member.

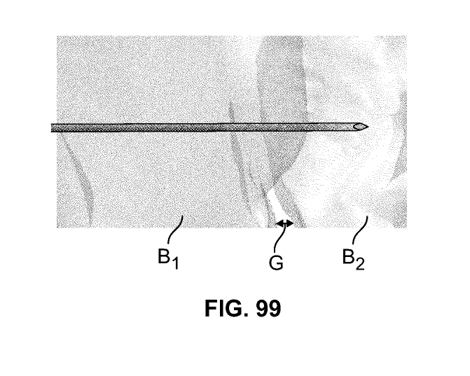

FIGS. 99-108 show an illustrative method for positioning a first bone relative to a second bone. The first bone may be an ilium. The second bone may be a sacrum. The method illustrated in FIGS. 99-108 may include additional method steps disclosed herein. The method may be performed in the order shown. The method may be performed in an order different from the order shown. The method may involve one or more of some or all of the apparatus shown in FIGS. 99-108.

The method illustrated in FIGS. 99-108 may be performed unilaterally or bilaterally. The method illustrated in FIGS. 99-108 may be performed from two, three or more positions extending across the sacroiliac joint.

FIG. 99 shows a guide wire piercing tissue B1 and B2 and traversing gap G. Gap G shows a distance between ilium I and sacrum S that is greater than a desired distance. A tip 9204 of the guide wire is placed where the distal end of distal cage 9805 is desired to be disposed. One or both of anterior-posterior lateral placement of the guide wire may be confirmed by medical imagery.

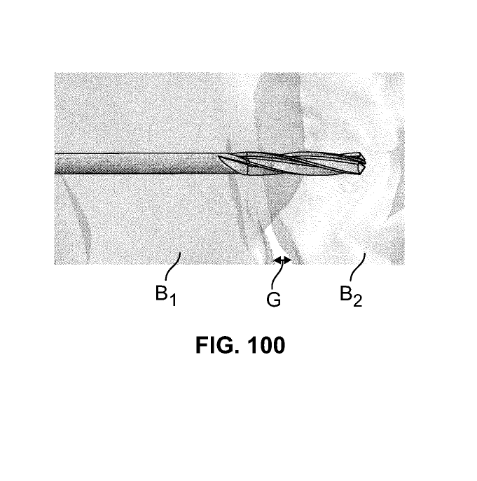

FIG. 100 shows a drill advanced along the path established by the guide wire (shown in FIG. 99). The drill may be cannulated, and may be advanced over the guide wire.

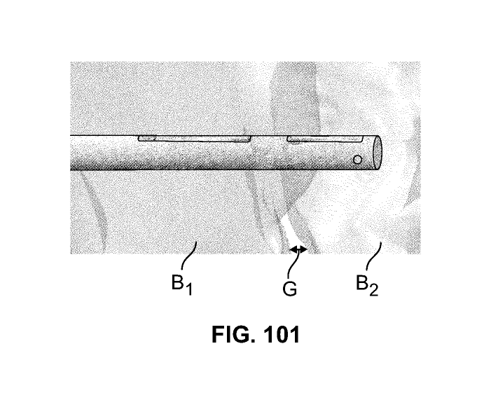

FIG. 101 shows a two-cavity preparation instrument. The device may have one or more features in common with the cavity preparation instrument, etc. The device is shown with dual broaching members, both in contracted state, one in tissue B1, the other in tissue B2, at locations selected for cages 9805 and 9807.

Cavity preparation may include rotating the cavity preparation instrument and enlarging the hole formed during the drilling to distract and remove bone and disc material to prepare two cavities. Each cavity may be shaped to receive a cage. The cage may be an expandable, implantable cage. The cage may have one or more features in common with the first cage. The cage may have one or more features in common with the second cage.

The device may include the broaching member. The broaching member may be a first expandable broaching member. The device may include a second expandable broaching member. The first expandable broaching member may be spaced apart from the second expandable broaching member. The device may be configured to prepare a first cavity in the sacrum. The first cavity may be prepared by activating the first broaching member. The device may be configured to prepare a second cavity in the ilium. The second cavity may be prepared by activating the second broaching member. The first broaching member may be activated together with the second broaching member. The first broaching member may be activated separately from the second broaching member.

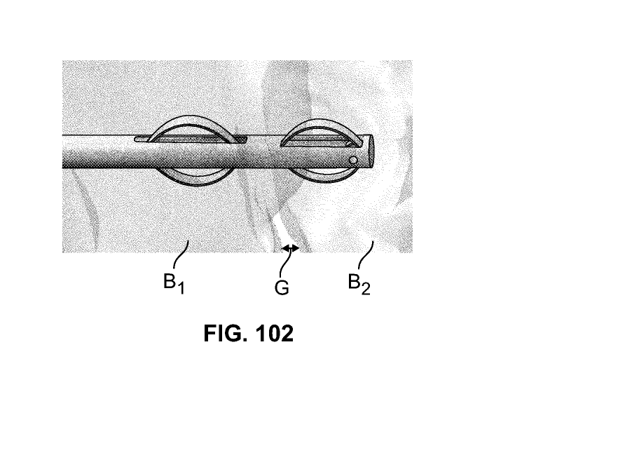

FIG. 102 shows the device with broaching members expanded for cavity preparation. Rotating the device may form a cavity in sacrum S and ilium I. The cavities in sacrum S and ilium I may be formed simultaneously. The cavity in sacrum S may be formed before or after forming the cavity in ilium I.

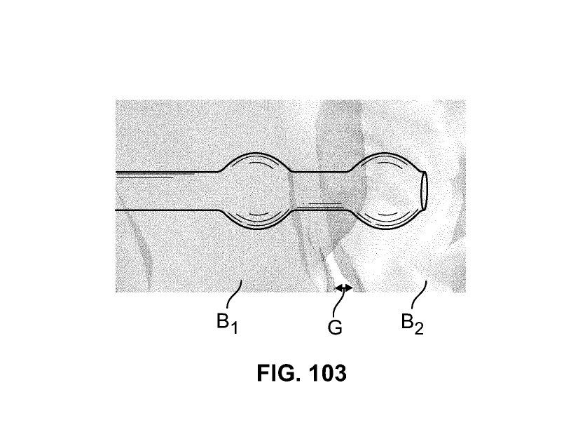

FIG. 103 shows cavities that may be prepared with the device, along with a path cleared by the drill. The cavities may have the same volume. The cavities may have different volumes.

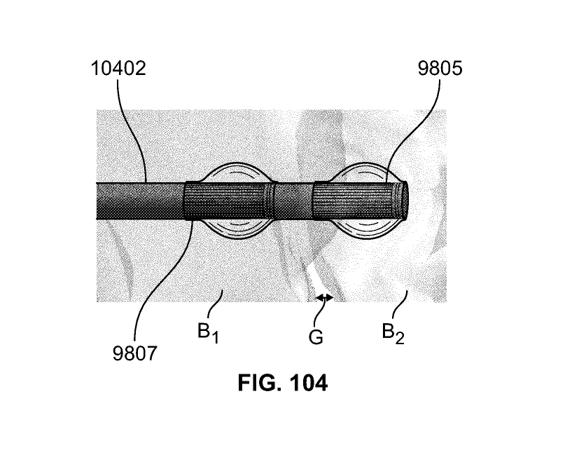

FIG. 104 shows proximal delivery tail 10402 positioning distal cage 9805 (shown, schematically, contracted) and proximal cage 9807 (shown, schematically, contracted), in their respective cavities.

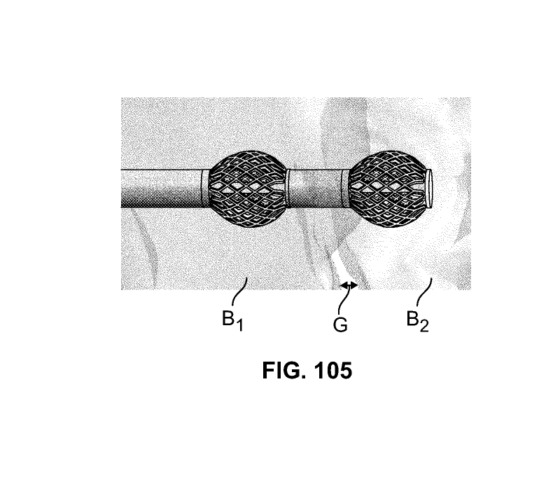

FIG. 105 shows cages expanded in the cavities, with assembly 9809, shown at initial length. (It will be appreciated that heretofore in the procedure, assembly 9809 may be maintained at initial length, though preceding illustrations may not show it as such.)

The cages may include one, two or more expandable laser-cut tubes. An expandable cage may include a plurality of expandable cells. An expandable cell may be configured to engage an anchor or screw. A geometric shape of an expandable layer may be defined by a cell density or other properties of cells in the layer. After any cage is expanded, a first layer may be spaced radially apart from a second layer. The second layer may buttress the first layer.

The cages may be rotated within the prepared cavity by engaging the handle. The rotating may expand the cages. Expanding the cages may further contour the cavity. Rotating the cages may expand one or both of the cages. Rotating the cages may contour one or both of the cavities.

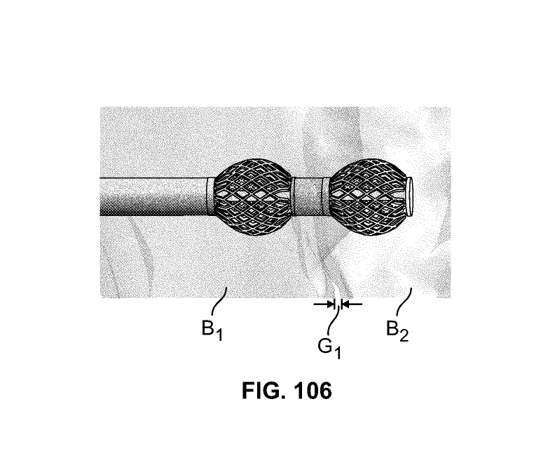

FIG. 106 shows assembly 9809 drawn down to a shorter length, and a concomitant shorter gap G. Gap G may be maintained in its reduced state by pressure of the cages against the cavity walls that results from the shortening if assembly 9809.

Material such as bone, biologics, or cement may be deposited into one or both of the cages. Material may be packed in the cages before and/or after deployment of the cages in the prepared cavities.

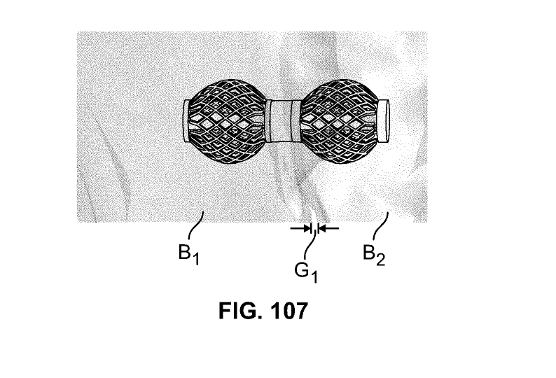

FIG. 107 shows apparatus 9800 after the removal of delivery tail 10402.

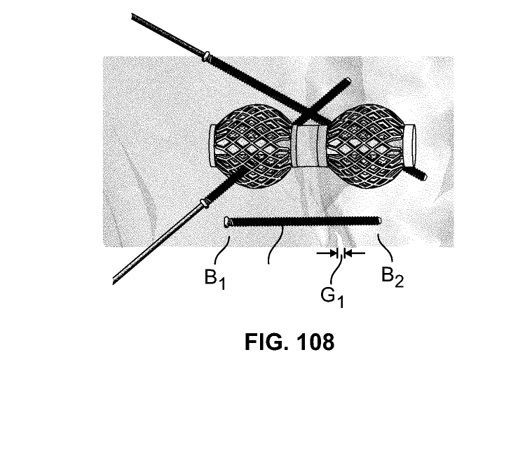

FIG. 108 shows anchors driven through the cages into tissue B1 and B2. An anchor is shown traversing gap G without passing through a cage.

Multiple anchors may be used to bridge the SI Joint in multiple locations and/or used in conjunction with screws to add stability. Screws may also be advanced through the cells of the implanted cages.

The apparatus and methods shown or described herein may include, or involve, cages having, in the expanded state, any suitable shape, including those shown or described above.

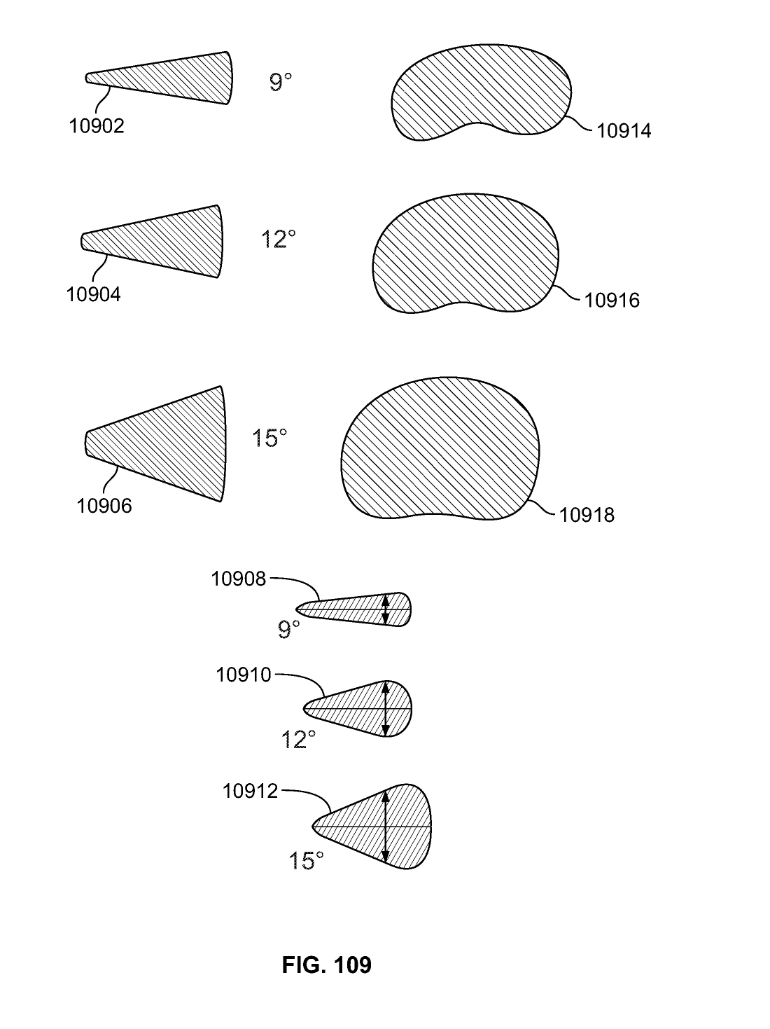

FIGS. 109-113 show other illustrative shapes, or cross-sections of shapes, of cages in the expanded state.

The cages may have one or more features in common with the first cage. The cages may have one or more features in common with the second cage. The first cage, when expanded, may have a shape or cross section illustrated in FIGS. 109-113. The second cage, when expanded, may have a shape or cross-section illustrated in one of FIGS. 109-113.

Different shapes may be appropriate for insertion in different spinal bodies or in different locations or orientations in a spinal body. During laser cutting of a tube, an expanded cage shape may be prepared by cutting larger mesh cells in regions of the tube where greater radial expansion is desired. Thermal shape setting may be used to pre-set a shape. A combination of thermal shape setting and cell size arrangement may be used to obtain the shape.

FIG. 109 shows schematic side views of illustrative taper angles that a cage may have. The short end of the taper may be placed anterior of the tall end. The short end of the taper may be placed posterior of the tall end. The taper direction may be arranged to run parallel to the anterior-posterior direction. The taper direction may arranged to run oblique to the anterior-posterior direction. Cage 10902 may have a 9° taper angle. Cage 10904 may have a 12° taper angle. Cage 10906 may have a 15° taper angle. Outlines 10908, 10910 and 10912 show cavity preparation instruments shaped to prepare cavities in spinal bodies that correspond to cages 10902, 10904 and 10906, respectively. Additional exemplary tapers are shown at Table 1, above.

FIG. 109 shows schematic top views of illustrative outlines that a cage may have. Outlines 10914, 10916 and 10918 are generally concave on one side and convex on other sides.

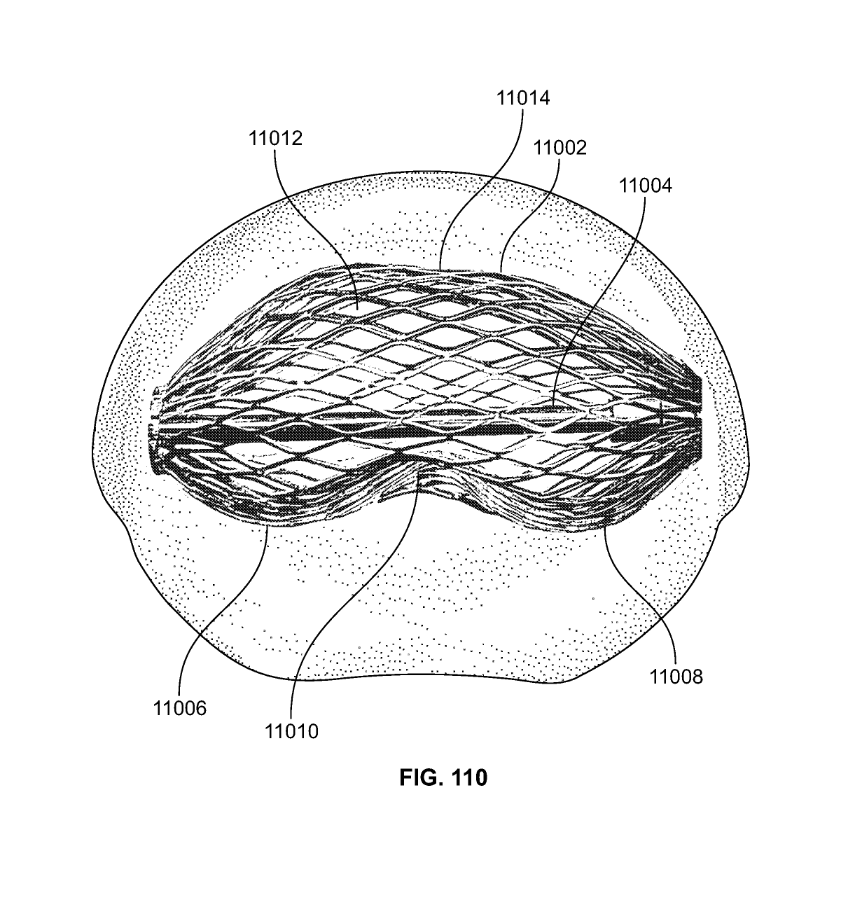

FIG. 110 shows illustrative cage 11002 positioned over the top of a vertebra (posterior at bottom of figure). Cage 11002 may have straight central axis member 11004. Upon expansion, cage 11002 may form lobes 11006 and 11008. A lobe may include a local three-dimensional maximum radial distance between the cage mesh and the central member. Concavity 11010 may be present between lobes 11006 and 11008. A concavity 11010 may include a local three-dimensional minimum radial distance between the cage mesh and the central member.

Upon expansion, cage 11002 may form lobe 11012. Lobe 11012 may include concavity 11014. Lobes may be positioned above or upon the vertebra to contour anatomy that is one or more of above, below or to the side of the cage.

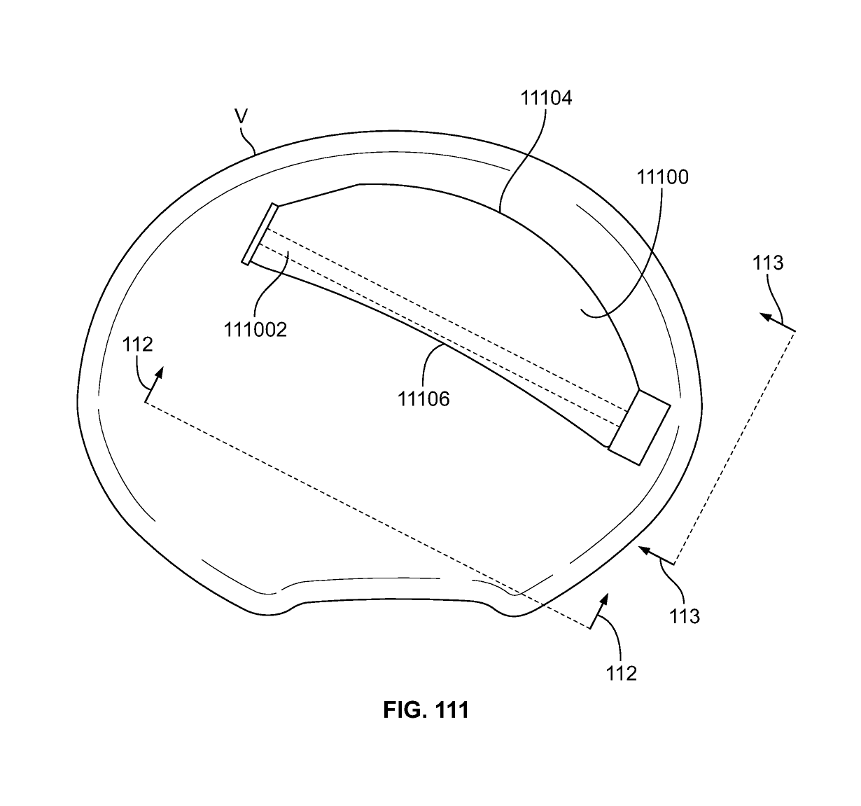

FIG. 111 shows, schematically, illustrative cage 11100 positioned over the top of vertebra V (posterior at bottom of figure). Cage 11100 may have straight central axis member 11102. Upon expansion, cage 11100 may form lobe 11104. Upon expansion, cage 11100 may form concavity 11106.

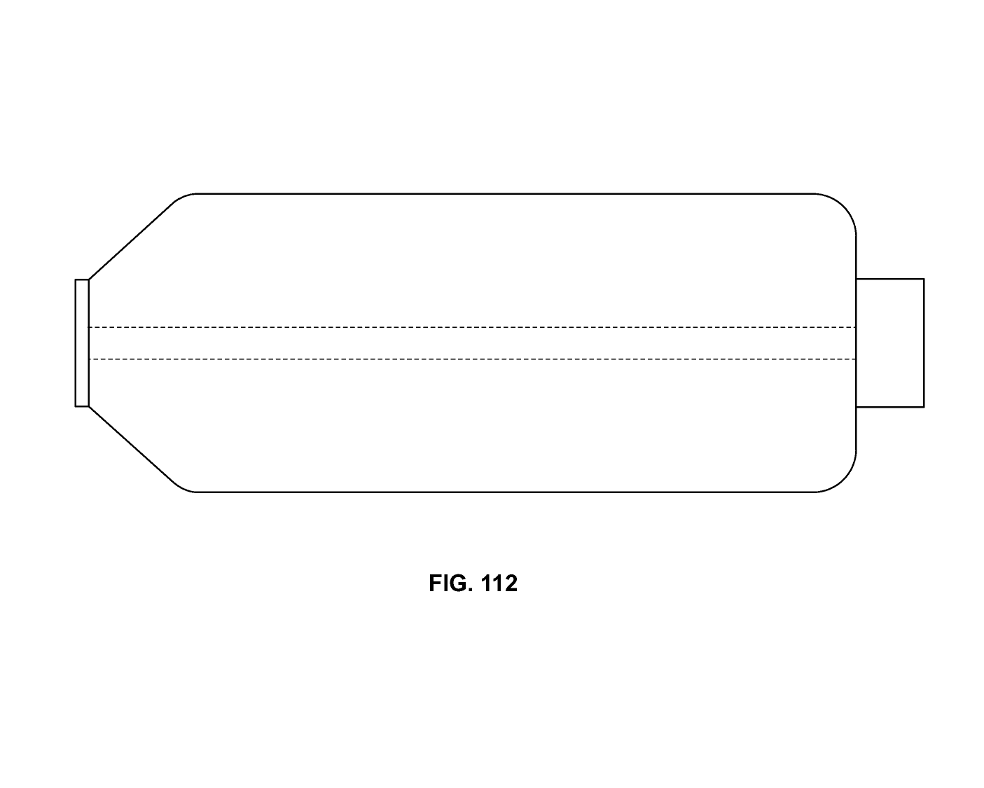

FIG. 112 shows, schematically, cage 1110, along lines 112-112 (shown in FIG. 111).

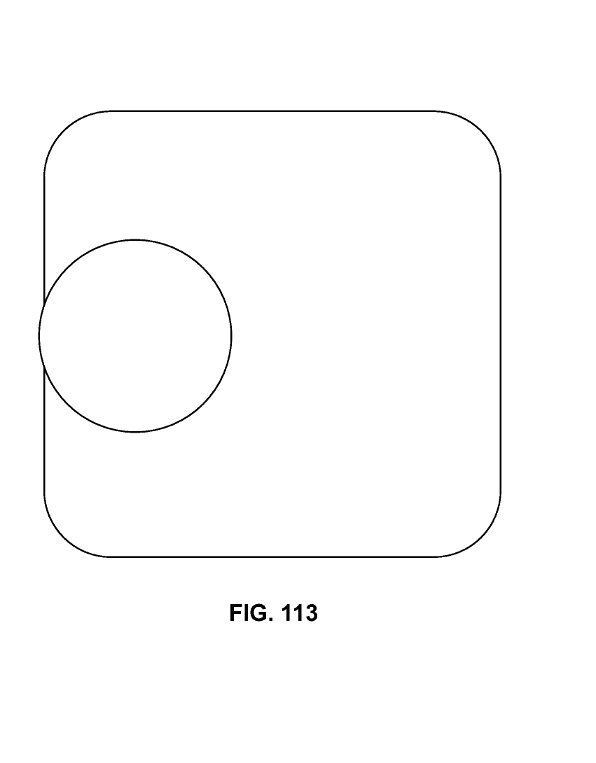

FIG. 113 shows, schematically, cage 1110, along lines 113-113 (shown in FIG. 111).

Thus, apparatus and methods for treating a spinal body have been provided. Apparatus and methods for treating a joint have also been provided. Persons skilled in the art will appreciate that the present invention can be practiced by other than the described examples, which are presented for purposes of illustration rather than of limitation. The present invention is limited only by the claims that follow.

1. A method for treating a spinal body having an interior, the method comprising:
augmenting a height of the spinal body by radially expanding a first mesh cage in the interior;
removing the first cage from the interior;
supporting the spinal body in an elevated position by radially expanding a second mesh cage in the interior; and
surgically enclosing the second cage in the interior.

2. The method of claim 1 wherein the spinal body is an intervertebral disc.

3-5. (canceled)

6. The method of claim 1 wherein the spinal body is a vertebra.

7-13. (canceled)

14. The method of claim 1 wherein the first mesh cage is expanded to a first volume and the second mesh cage is expanded to a second volume that is greater than the first volume.

15. The method of claim 1 further comprising forming a cavity in the interior by rotating a cavity preparation instrument about a central axis of the cavity preparation instrument.

16. (canceled)

17. The method of claim 15 wherein:
the cavity has a first volume; and
the second cage is the radially expanded to a second volume that is greater than the first volume.

18-19. (canceled)

20. The method of claim 15 further comprising forming a second cavity in the interior by rotating a second cavity preparation instrument about a central axis of the second cavity preparation instrument in the interior, wherein:
the cavity is a first cavity and has a first volume;
the broaching member is a first broaching member;
the second cavity has a second volume greater than the first volume;
the forming the first cavity is performed before expanding the first mesh and before expanding the second mesh; and
the forming the second cavity is performed after expanding the first mesh and before expanding the second mesh.

21. (canceled)

22. The method of claim 15 wherein the cavity preparation instrument includes a broaching member and the forming the cavity includes radially expanding the broaching member in the interior.

23-24. (canceled)

25. The method of claim 15 wherein:
the cavity preparation instrument includes a broaching member;
the spinal body is an intervertebral disc and is disposed between a first vertebra and a second vertebra; and
the forming the cavity further comprises rotating the broaching member in the interior to scrape a first vertebral endplate disposed between the intervertebral disc and the first vertebra and a second vertebral endplate disposed between the intervertebral disc and the second vertebra.

26. The method of claim 2 wherein the augmenting further comprises:
positioning a broaching member in the interior;
positioning the broaching member against a first vertebra and a second vertebra, the intervertebral disc being disposed between the first vertebra and the second vertebra; and
expanding the broaching member.

27. The method of claim 6 wherein the augmenting further comprises:
positioning a broaching member in the interior;
positioning the broaching member against the vertebra; and
expanding the broaching member.

28-29. (canceled)

32. The method of claim 1 further comprising seating the second mesh cage in the interior by rotating the second mesh cage.

33. The method of claim 32 wherein the rotating further comprises depositing bone in an interior of the second mesh cage.

34-35. (canceled)

36. The method of claim 1 wherein the radially expanding the first mesh cage includes radially expanding a first laser-cut tube to form a first shape and radially expanding a second laser-cut tube to form a second shape within the first shape, the second tube, in the second shape, buttressing the first tube.

37. The method of claim 1 wherein the radially expanding the first mesh cage includes:
radially expanding a first laser-cut tube to form a first shape;
radially expanding a second laser-cut tube to form a second shape within the first shape, the second tube, in the second shape, buttressing the first tube; and
radially expanding a third laser-cut tube to form a third shape within the second shape, the third tube, in the third shape, buttressing the first and second tubes.

38-45. (canceled)

46. The method of claim 2 wherein the supporting further comprises advancing a screw through a vertebra and into a cell included in the second mesh cage, the vertebra being disposed adjacent the intervertebral body.

47. The method of claim 2 wherein the supporting, further comprises:
positioning a plate on a vertebra disposed adjacent the intervertebral body; and
advancing a screw through the plate and the vertebra and into a cell included in the second mesh cage.

48. (canceled)

49. The method of claim 2 wherein the supporting further comprises advancing a screw through a first vertebra, a first cell and a second cell, and into a second vertebra;
wherein:
the intervertebral body is disposed between the first vertebra and the second vertebra; and
the second cage includes the first cell and the second cell.

50. The method of claim 2 wherein the supporting further comprises advancing a screw through a first vertebra, through the second mesh cage and into a second vertebra;
wherein the intervertebral body is disposed between the first vertebra and the second vertebra.

51. (canceled)

52. The method of claim 2 further comprising:
inserting a first screw into a first vertebra;
expanding a distal end of the first screw to form a third mesh cage;
inserting a second screw into a second vertebra; and
expanding a distal end of the second screw to form a fourth mesh cage;
wherein the intervertebral body is disposed between the first vertebra and the second vertebra.

53-135. (canceled)
